(19) United States
(12) Patent Application Publication
(10) Pub. No.: US 2023/0123585 A1
(43) Pub. Date: Apr. 20, 2023

(54) COMPOSITIONS AND METHODS FOR TREATING OR PREVENTING CANCER USING DEUBIQUITINASE INHIBITORS

(71) Applicant: The University of Chicago, Chicago, IL (US)

(72) Inventors: Chuan He, Chicago, IL (US); Hui-Lung Sun, Chicago, IL (US); Jiangbo Wei, Chicago, IL (US); Allen C. Zhu, Chicago, IL (US); Huanyu Wang, Chicago, IL (US)

(21) Appl. No.: 17/905,384

(22) PCT Filed: Mar. 3, 2021
(86) PCT No.: PCT/US2021/020653

§ 371 Date: Aug. 31, 2022

Related U.S. Application Data
(60) Provisional application No. 62/984,679, filed on Mar. 3, 2020.
(60) Provisional application No. 63/040,080, filed on Jun. 17, 2020.

(51) Int. Cl. A61K 38/14; A61K 31/194; A61K 31/4184; A61K 31/502; A61K 31/513; A61K 31/5377; A61K 31/551; A61K 45/06; A61P 35/00

(52) U.S. Cl. CPC A61K 38/14; A61K 31/194; A61K 31/4184; A61K 31/502; A61K 31/513; A61K 31/5377; A61K 31/551; A61K 45/06; A61P 35/00

(57) ABSTRACT

The present disclosure provides novel materials and methods related to the treatment of cancer. In particular, the present disclosure provides compositions and methods for treating and/or preventing cancer based on the attenuation of Methyltransferase-like Protein 3 (METTL3) activity in a tumor cell. The compositions and methods disclosed herein include the use of a deubiquitinase inhibitor with or without an agent that modulates chromatin state and/or an agent that modulates DNA damage repair.

### CROSS REFERENCE To RELATED APPLICATIONS

This application claims priority to and the benefit of U.S. Provisional Patent Application No. 62/984,679 filed Mar. 3, 2020, and U.S. Provisional Patent Application No. 63/040,080 filed Jun. 17, 2020, both of which are incorporated herein by reference in their entirety and for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 6,151 Byte ASCII (Text) file named “2021-03-03_38142-601_SQL_ST25.txt,” created on Mar. 3, 2021.

### FIELD

The present disclosure provides novel materials and methods related to the treatment of cancer. In particular, the present disclosure provides compositions and methods for treating and/or preventing cancer based on the attenuation of Methyltransferase-like Protein 3 (METTL3) activity in a tumor cell. The compositions and methods disclosed herein include the use of a deubiquitinase inhibitor with or without an agent that modulates chromatin state and/or an agent that modulates DNA damage repair.

### BACKGROUND

Diffuse Embryonic stem cells (ESCs) are derived from the inner cell mass of the pre-implantation blastocyst. Under appropriate in vitro culture conditions, ESCs proliferate indefinitely without differentiation, a property referred to as self-renewal, and at the same time retain the developing potential to generate cells of three primary germ layers, known as pluripotency. Studies of ESCs hold promise for tissue repair and provide a potential tool for modeling human disease. In order to fulfill the potential of ESCs, it is critical to understand how ESCs are regulated. The differentiation depends on many regulators that control gene expression, including DNA methylation, transcription factors, and histone/RNA modifications. Mouse, rat, and human ESCs, for instance, share a common subset of transcription factors that specify “stemness”, which include Oct4, Sox2, and Nanog. Signaling pathways have also been shown to regulate ESC fate determination. The JAK/STAT3, ERK, Wnt, and TGF pathways all play roles in affecting downstream gene regulation. For example, ERK signaling guides ESCs to exit pluripotency by phosphorylation of transcription factors that ultimately inhibit expression of genes that maintain pluripotency. Current efforts further seek to elucidate the molecular regulators and signaling pathways that maintain proper differentiation of ESCs.

Recent studies have shown that messenger RNA (mRNA) modifications play a critical role in regulating stem cell differentiation and animal development. Among over 150 known RNA modifications, N⁶-methyladenosine(m⁶A) is an evolutionarily conserved and the most abundant internal mRNA modification in most eukaryotic mRNA. m⁶A is reversibly, site-selectively installed on mRNA transcripts by “writers,” with a portion that can be removed by “erasers.” The m⁶A methyltransferase “writer” complex has a heterodimeric core made up of the catalytic component METTL3 and its binding partner METTL14; it also includes a co-factor WTAP (Wilms' Tumor 1-Associating Protein). Meanwhile, “eraser” proteins FTO and ALKBHS remove the m⁶A modification.

m⁶A can not only affect RNA secondary structure, but also be recognized by m⁶A “reader” proteins, which exert effects on mRNA metabolism and translation. These m⁶A-dependent functions include translation initiation, RNA decay, and splicing. It is not surprising, then, that m⁶A has emerged as a main regulator of gene expression, particularly during development and cell differentiation. In particular, METTL3 has been found to play an essential role in early development. Loss of METTL3 in mouse embryonic cells depletes m⁶A and increases stability of certain transcripts such as Nanog. This impedes decay of pluripotency factors that maintain self-renewal, thereby also delaying proper lineage priming and fate transition, leading to early embryo lethality. Depletion of the Drosophila METTL3 homolog Ime4 prevents proper Sexually splicing and thus leading to failure of sex determination. These studies have shown that m⁶A methylation controls stability of transcripts, including those that promote naïve pluripotency and require timely downregulation for proper differentiation, and that m⁶A deposition is crucial for the temporal regulation of development. The importance of m⁶A methylation has been well described recently, yet gaps in the understanding of how this process is regulated remain.

### SUMMARY

Embodiments of the present disclosure include a composition for attenuating Methyltransferase-like Protein 3 (METTL3) in a tumor cell. In accordance with these embodiments, the composition includes at least one deubiquitinase inhibitor, at least one chromatin state modulator and/or at least one DNA damage modulator, and a pharmaceutically acceptable carrier or excipient.

In some embodiments, the at least one deubiquitinase inhibitor targets Ubiquitin Carboxyl-terminal Hydrolase 5 (USP5). In some embodiments, the at least one chromatin state modulator includes a bromodomain and extraterminal domain (BET) inhibitor, a histone methyl transferase (HMT) inhibitor, and/or a poly(ADP-ribose) polymerase 1 (PARP1) inhibitor. In some embodiments, the at least one DNA damage repair modulator induces DNA damage and/or inhibits DNA repair.

In some embodiments, inhibiting USP5 attenuates METTL3 protein stability and/or activity.

In some embodiments, the at least one deubiquitinase inhibitor comprises EOAI3402143, vialinin, WP1130, mebendazole, PYR-41, gossypetin, formonectin, suramin, and combinations thereof. In some embodiments, the BET inhibitor comprises a thienotriazolodiazepine, OTX015, BET-d246, ABBV-075, I-BET 151, I-BET 762, CPI 203, PFI-1, RVX-208, Dinaciclib, and combinations thereof. In some embodiments, the thienotriazolodiazepine is JQ1.

In some embodiments, the HMT inhibitor comprises chaetocin, GSK343, UNC199, SGC0946, F5446, Pinometostat, EPZ004777, EPZ005687, tazemestostat, JQEZ5, CPI-1205, EPZ001989, EBI-2511, PF-06726304, El 1, GSK503, GSK126, CPI-169, ZLD 1039, SAH-EZH2, NSC 617989, CPI-169, CPI-360, EPZ6438, and combinations thereof.

In some embodiments, the PARP inhibitor comprises olaparib, rucaparib, veliparib, talazoprib, AG-14361, INO-1001, A-966492, PJ34, Niraparib, UPF 1069, ME0328, Pamiparib, NMS-P118, E7449, picolinamide, Benzamide, Nu1025, Iniparib, AZD2461, BGP-15, and combinations thereof.

In some embodiments, the at least one DNA damage repair modulator comprises bleomycin, 5-FU, ceralasertib (AZD6738), cisplatin, oxaliplatin, carboplatin, Cytoxan, and combinations thereof.

In some embodiments, the composition comprises at least one deubiquitinase inhibitor and wherein the at least one chromatin state modulator is a BET inhibitor. In some embodiments, the composition comprises at least one deubiquitinase inhibitor and wherein the at least one chromatin state modulator is a HMT inhibitor. In some embodiments, the composition comprises at least one deubiquitinase inhibitor and wherein the at least one chromatin state modulator is a PARP inhibitor. In some embodiments, the composition comprises at least one deubiquitinase inhibitor and at least one DNA damage repair modulator.

Embodiments of the present disclosure also include a method of treating or preventing cancer in a subject comprising administering any of the pharmaceutical compositions described above. In some embodiments, a method of treating or preventing cancer in a subject includes administering a composition comprising at least one deubiquitinase inhibitor, and at least one of a bromodomain and extraterminal domain (BET) inhibitor, a histone methyl transferase (HMT) inhibitor, a poly(ADP-ribose) polymerase 1 (PARP1) inhibitor, and/or a DNA damage repair modulator.

In some embodiments of the method, the composition further comprises a pharmaceutically acceptable carrier or excipient, and wherein the composition is administered to a subject diagnosed with cancer.

In some embodiments of the method, the at least one deubiquitinase inhibitor comprises EOAI3402143, vialinin, WP1130, mebendazole, PYR-41, gossypetin, formonectin, suramin, and combinations thereof.

In some embodiments of the method, the BET inhibitor comprises a thienotriazolodiazepine, OTX015, BET-d246, ABBV-075, I-BET 151, I-BET 762, CPI 203, PFI-1, RVX-208, Dinaciclib, and combinations thereof.

In some embodiments of the method, the HMT inhibitor comprises chaetocin, GSK343, UNC199, SGC0946, F5446, Pinornetostat, EPZ004777, EPZ005687, tazemestostat, JQEZ5, CPI-1205, EPZ001989, EBI-2511, PE-06726304, El1, GSK503, GSK126, CPI-169, ZLD 1039, SAH-EZH2, NSC 617989, CPI-169, CPI-360, EPZ6438, and combinations thereof.

In some embodiments of the method, the PARP inhibitor comprises olaparib, rucaparib, veliparib, talazoprib, AG-14361, INO-1001, A-966492, PJ34, Niraparib, UPF 1069, ME0328, Pamiparib, NMS-P118, E7449, picolinamide, Benzamide, Nu1025, Iniparib, AZD2461, BGP-15, and combinations thereof.

In some embodiments of the method, the composition attenuates METTL3 stability and/or activity and induces apoptosis of a cancer cell.

In some embodiments of the method, the composition comprises at least one deubiquitinase inhibitor and at least one BET inhibitor. In some embodiments of the method, the combination of the at least one deubiquitinase inhibitor and the at least one BET inhibitor exhibits a synergistic effect on cancer cell viability.

In some embodiments of the method, the composition comprises at least one deubiquitinase inhibitor and at least one HMT inhibitor. In some embodiments of the method, the combination of the at least one deubiquitinase inhibitor and the at least one HMT inhibitor exhibits a synergistic effect on cancer cell viability.

In some embodiments of the method, the composition comprises at least one deubiquitinase inhibitor and at least one PARP inhibitor. In some embodiments of the method, the combination of the at least one deubiquitinase inhibitor and the at least one PARP inhibitor exhibits a synergistic effect on cancer cell viability.

In some embodiments of the method, the combination of the at least one deubiquitinase inhibitor and the at least one DNA damage repair modulator or inhibitor that blocks DNA damage repair exhibits a synergistic effect on cancer cell viability.

In some embodiments of the method, the cancer is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, glioblastoma, lymphoma, and leukemia.

### BRIEF DESCRIPTION OF THE DRAWINGS

FIGS. 1A-1D: ERK Activation Promotes mRNA m⁶A Methylation. (A) Schematic diagram of a circular RNA (circRNA) translation reporter consisting of a single exon and two introns with complementary sequences. The exon containing GGACU can be back-spliced to generate circRNAs that drive GFP translation. (B) Overview of CRISPR screening. Cas9 knockout libraries are packaged into lentivirus and then transduced into HeLa cells contain circRNA GFP reporters. Cells with the top and bottom 5% GFP expression are collected by flow cytometry. The sgRNA are amplified from genomic DNA and then analyzed by next-generation sequencing followed by statistical analyses to identify candidate genes. (C) Positive regulators for the m⁶A pathway identified in the CRISPR screening using circular GGACU-GFP reporters. (D) Pathway analysis of sgRNA enriched in the bottom 5% GFP cells with circRNA GFP reporters. (See also FIG. 8; Table 1.)

FIGS. 2A-2F: ERK Interacts and Phosphorylates METTL3 and WTAP. (A) Sequence alignment of the conserved D-domain on METTL3 and WTAP predicted by the eukaryotic linear motif website. The D domain possess a consensus binding sequence of (Lys/Arg)0-2-(X)1-6-Φ-X-Φ: where Φ is a hydrophobic residue such as Leu, Ile, Val, Phe, and X is any amino acid. (B) Interaction between wild-type (WT) or mutant METTL3, WTAP and ERK2 in lysates from B-RAF-expressing 293T cells transfected as indicated was examined by co-immunoprecipitation (IP). EE, R415E/R416E METTL3 or R71E/R72E WTAP. (C) Sequence alignment of the conserved serine residues on METTL3 that are phosphorylated by ERK. (D) Phos-tag SDS-PAGE showing the phosphorylation status of WT or non-phosphorylatable alanine mutants of METTL3 in 293T cells co-transfected with B-RAF. 2A, T43A/S50A; 3A, S43A/S50A/S525A. (E) Sequence alignment of the serine/threonine-proline (S/T-P) motif on WTAP. (F) Phos-tag SDS-PAGE showing the phosphorylation status of WT or non-phosphorylatable alanine mutants of human WTAP in 293T cells co-transfected with B-RAF. 2A, S306A/S341A. (See also FIG. 9 and FIG. 10.)

FIGS. 3A-3G: USP5 is Required for ERK-Mediated METTL3 Stabilization. (A) Comparison of METTL3 and WTAP protein levels in mESCs and A375 stable transfectants by immunoblotting (IB). (B) 293T cells transfected as indicated were treated with MG-132 (10 μM, 6 h) followed by IP/IB analysis. (C) 293T cells transfected with WT or 3A METTL3 for 48 h, followed by cycloheximide (CHX) 10 μg/ml for 0-6 h. Cell lysates were used for immunoblotting to measure the protein levels of METTL3. (D) USP5 was identified as a positive regulator in the CRISPR screening using circular GGACU-GFP reporters. (E) 293T cells transfected as indicated were treated with MG-132 (10 μM, 4 h) followed by IP/IB assay. (F) Overexpression of B-RAF and USP5 increases METTL3 expression. Lysates of 293T transfected as indicated were analyzed by immunoblot. (G) Knockdown of USP5 in A375 cells decreases METTL3 expression. See also FIG. 11.)

FIGS. 4A-4E: Phosphorylation of METTL3/WTAP by ERK Facilitates Resolution of Pluripotency. (A) LC-MS/MS quantification of the m⁶A/A ratio in mRNA of mESC stable transfectants. (B) Representative flow cytometry analyses of mESC stable transfectants for the activity of alkaline phosphatase (AP) and expression of stage-specific embryonic antigen-1 (SSEA-1). (C) qPCR analysis of pluripotency genes in R-3A2A versus R-WT mESCs. (D) Relative levels of Nanog, measured by qPCR, at the indicate times after 5 μg/ml actinomycin D treatment. mRNA levels were monitored in R-WT (black), R-WT with 10 μM PD0325901 (green), and R-3A2A (red) mESC. (E) qPCR analysis for pluripotency and differentiation markers expression after 8 days of embryonic body (EB) induction. Error bars indicated SD (n=3). (See also FIG. 12.)

FIGS. 5A-5G: Transcripts Affected by Phosphorylation of Methyltransferase Complex in mESCs. (A) Cumulative distribution function of 1og2 peak intensity of m⁶A-modified sites in R-WT and R-3A2A mESCs. (B) Volcano plot for peaks with differential m⁶A intensity between R-WT and R-3A2A mESCs. Fold change (FC) is the ratio of IP over Input for R-WT and R-3A2A. (C) Coverage plots of m⁶A peaks in the Nanog, Lefty1, and Zfp219 comparing R-WT and R-3A2A mESCs. Plotted coverages are the medians of three replicates. (D) Gene enrichment analysis with WikiPathway terms of differentially m⁶A methylated peaks in R-WT and R-3A2A mESCs for molecular functions. (E) GSEA analysis on enrichment of histone binding protein in R-3A2A versus R-WT mESCs. (F) ELISA analysis for histone post-translational modifications of histone extracts from mESCs. Bars represent the ratio of R-3A2A relative to R-WT mESC. Red color was used to highlight a ratio greater than 2. (G) Gene enrichment analysis with WikiPathway terms of differentially expressed genes (p<0.05). (See also FIG. 13; data relating to genes identified by GSEA related to histone binding proteins, and data relating to significantly altered m6A peaks between R-WT and R-3A2A mESCs can be made available upon request.)

FIGS. 6A-6H: Phosphorylation of the m⁶A Methyltransferase Complex May Affect Tumorigenesis. (A) Lysates of A375 stable transfectants harvested at different time points after treatment with cycloheximide (CHX) 10 μg/ml were analyzed by immunoblot. (B) LC-MS/MS quantification of the m⁶A/A ratio in mRNA of A375 stable transfectants. (C) After 8 h treatment with 10 μM PD0325901 or 0.1 μM tramentib, cell lysates from A375 cells were analyzed by immunoblot. (D) LC-MS/MS quantification of the m⁶A/A ratio in mRNA of A375 cells treated with 10 μM PD0325901 or 0.1 μM tramentib for 48 h. (E) After 8hr treatment with 10 μM EOAI3402143 (EOAI) or 30 μM vialinin A , cell lysates from A375 cells were analyzed by immunoblot. (F) A375 stable transfectants as indicated were treated with 3 μM EOAI3402143 (EOAI) or 10 μM vialinin A before measuring cell viability by SRB assay. Data are presented as relative to the R-WT cells without drug treatment (n=3 per group, data represent mean±SEM). (G) Immunofluorescence analysis of METTL3 (green) in SKBR3 cells treated with 1 μM tucatinib and 1 μM lapatinib for 8 hr. DAPI (blue) was used to mark the nucleus. (H) LC-MS/MS quantification of the m⁶A/A ratio in mRNA of SKBR3 and BT474 cells treated with 1 μM tucatinib and 1 μM lapatinib for 48 h. (See also FIG. 14.)

FIG. 7: Schematic summary depicting the role of the m⁶A Methyltransferase Phosphorylation by ERK.

FIGS. 8A-8D: ERK Activation Promotes mRNA m⁶A Methylation. (A) Representative flow cytometry analyses of HeLa circular-GFP reporter cells transfected with or without METTL3 for 48 h. (B) Lysates of 293T cells transfected with the m⁶A writer complex and ERK-activated kinase were analyzed by SDS-PAGE or phos-tag SDS-PAGE. (C) Lysates of 293T cells transfected with METTL3 and 13 different oncogenic kinases were analyzed by SDS-PAGE or phos-tag SDS-PAGE. (D) 293T cells were transfected with a luciferase reporter containing NANOG 3′UTR (pLightswtich-nanog), m⁶A writer complex, and ERK-activated kinase for 48 h before luciferase assay. Data are presented relative to the cells transfected only pLightswtich-NANOG (n=3 per group, data represent mean±SEM, p values were calculated by Student's t test).

FIGS. 9A-9C: ERK Interacts and Phosphorylates METTL3 and WTAP. (A) Lysates of 293T cells transfected as indicated were subjected to IP with anti-Flag antibody followed by immunoblot. (B) Lysates of 293T cells transfected as indicated were subjected to IP with anti-Flag antibody followed by immunoblot. (C) Immunofluorescence analysis of 293T cells co-transfected with myc-METTL3 or WTAP (green), Flag-USP5 (red) with or without constitutively active B-RAF.

FIGS. 10A-10G: ERK Interacts and Phosphorylates METTL3 and WTAP. (A-C) Mass spectrometry detected S43, S50 and S525 phosphorylation in METTL3 in 293T cells co-transfected with B-RAF V600E. (D) Characterization of anti-p-METTL3 (S43) antibodies of 293T cells transfected as indicated with MEK S218D/S222D, HER2 V659E, B-RAF V600E and WT or non-phosphorylatable alanine mutant (3A) METTL3. (E) The METTL3 S43 phosphorylation was identified by in vitro kinase assays, in which purified METTL3-METTL14 were incubated with activated ERK2. (F) A375 cells were treated with 1 μM dabrafenib, 1 μM PLX-4720, 10 μM PD0325901, 0.1 μM tramentib for 1 h. Cell lysates were subjected to IP with METTL3 antibody followed by immunoblot. (G) Phos-tag SDS-PAGE showing the phosphorylation status of WT or non-phosphorylatable alanine mutants of mice WTAP in 293T cells co-transfected with B-RAF. 2A, T298A/S341A.

FIGS. 11A-11G: USP5 is Required for ERK-Mediated METTL3 Stabilization. (A) A375 cells transfected with HA-ubiquitin (ub) were treated with 10 μM MG-132 and MEK inhibitor PD0325901 for 8 h. The ubiquitination of METTL3 was detected by IP with anti-METTL3 and immunoblot with anti-HA. (B) After 8 h treatment with 10 μM PD0325901 with or without 10 μM MG-132, cell lysates from A375 cells were analyzed by immunoblot. (C) A375 cells were treated with various concentrations of PD0325901 for 1 h. Cell lysates were subjected to IP with METTL3 antibody followed by immunoblot. (D) 293T cells transfected as indicated were subjected to IP/immunoblot analysis. (E) Immunofluorescence analysis of myc-METTL3 (green) in mESC stable transfectants. DAPI (blue) was used to mark the nucleus. (F) Lysates of 293T cells transfected as indicated were subjected to IP with anti-Flag antibody followed by immunoblot. (G) Immunofluorescence analysis of 293T cells co-transfected with myc-METTL3 (green), Flag-USP5 (red) with or without constitutively active B-RAF.

FIGS. 12A-12I: Phosphorylation of METTL3/WTAP by ERK Facilitates Resolution of Pluripotency. (A) Representative phase-contrast microscopy showing colony size of R-3A2A versus R-WT mESC cells. (B) Cell growth of R-WT and R-3A2A mESCs were measured by sulforhodamine B dye (SRB assay). Data are presented as relative to the day 1 (n=3 per group, data represent mean±SEM). (C) meRIP-qPCR of pluripotency transcripts in R-3A2A versus R-WT mESCs. (D-H) Relative levels of Zfp42, Klf2, Sox2, Lefty1, and Pou5f 1, measured by qPCR, at the indicate times after 5 μg/ml actinomycin D treatment. mRNA levels were monitored in R-WT (black), R-WT with 10 μM PD0325901 (green), and R-3A2A (red) mESCs. (I) Representative phase contrast microscopy showing EB differentiation of R-WT and R-3A2A mESCs after 8 days.

FIGS. 13A-13F: Transcripts Affected by Phosphorylation of Methyltransferase Complex in mESCs. (A) Metagene plots showing the average distribution of m⁶A peaks identified across mRNA or lncRNA in the R-WT and R-3A2A mESCs. (B) Consensus sequence motifs among m⁶A peaks in R-WT and R-3A2A mESCs. (C) Overrepresentation analysis of genes with differentially m⁶A in R-WT and R-3A2A mESCs that overlapped with targets of transcriptional factors. (D) Distance matrix of the m⁶A methylation in replicates of R-WT and R-3A2A mESCs. (E) Higher m⁶A in the pluripotency gene (PluriNetwork) of R-WT mESCs. (F) Coverage plots of m⁶A peaks in Ezh1, Suz12, Set, and Mtf2 comparing R-WT and R-3A2A mESCs. Plotted coverages are the medians of three replicates.

FIGS. 14A-14I: Phosphorylation of the m⁶A Methyltransferase Complex May Affect Tumorigenesis. (A) Oncogenes (promote cancer), tumor suppressors (inhibit carcinogenesis), and drivers (important in cancer development, either oncogene or tumor suppressor) were used as classifiers by Cancermine database to identify the potential role of METTL3 from the published literature. (B) LC-MS/MS quantification of the m⁶A/A ratio in mRNA of melanoma cell lines. (C) After 8 h treatment with 10 μM PD0325901 or 0.1 μM tramentib, cell lysates from HCT-116 cells were analyzed by immunoblot. (D) Kaplan-Meier analysis of overall survival time based on METTL3 expression from the skin cutaneous melanoma (SKCM) dataset at The Cancer Genome Atlas (TCGA). (E) A375 cells transfected with HA-ubiquitin (ub) were treated with 10 μM MG-132 and 10 μM EOAI3402143 (EOAI) or 30 μM vialinin A for 8 h. The ubiquitination of METTL3 was detected by IP with anti-METTL3 and immunoblot with anti-HA. (F) After 8 hr treatment with 10 μM EOAI3402143 (EOAI) or 30 μM vialinin A, cell lysates from HCT-116 cells were analyzed by immunoblot. (G) HCT-116 stable transfectants as indicated were treated with 3 μM EOAI3402143 (EOAI) or 10 μM vialinin A before measuring cell viability by SRB assay. Data are presented as relative to the R-WT cells without drug treatment (n=3 per group, data represent mean±SEM). (H) Phos-tag SDS-PAGE showing the phosphorylation status of METTL3, METTL14, or WTAP in 293T cells co-transfected without or with HER2. (I) LC-MS/MS quantification of the m⁶A/A ratio in mRNA of breast cancer cell lines.

FIGS. 15A-15D: The results of the inhibition rates of JQ1 in different PDAC cell lines. (B) The correlation analysis of gene expression of BRD family genes and METTL3 in PDAC from TGCA and GTEx database. (C) The polyA RNA m6A level changes upon JQ1 treatment compared to the DMSO control in different PDAC cell lines. (D) The cell viability changes with JQ1 treatment with a series concentrations in the JQ1-insensitive cells upon the knockdown of METTL3 and the JQ1-sensitive cells upon the overexpression of METTL3 compared to control.

FIGS. 16A-16B: (A) The protein level changes of METTL3 with JQ1 treatment in the JQ1-insensitive cells compared to DMSO control (left); the RNA level changes of METTL3 the JQ1-insensitive cells with JQ1 treatment compared to DMSO control (mid); the polyA RNA m6A level changes in JQ1-insensitive cells among the knockdown control and DMSO control samples, the knockdown control and JQ1 treatment samples, the METTL3 knockdown and DMSO control, and the METTL3 knockdown and JQ1 treatment samples (right). (B) The protein level changes of METTL3 with JQ1 treatment in the JQ1-sensitive cells compared to DMSO control (left); the RNA level changes of METTL3 the JQ1-sensitive cells with JQ1 treatment compared to DMSO control (mid); the polyA RNA m6A level changes in JQ1-sensitive cells among the knockdown control and DMSO control samples, the knockdown control and JQ1 treatment samples, the METTL3 knockdown and DMSO control, and the METTL3 knockdown and JQ1 treatment samples (right).

FIGS. 17A-17I: (A-B) ELISA analysis for histone H3 post-translational modifications of A375 and HCT116 cells. Bars represent the ratio of METTL3 knockdown relative to wild type (WT) cells. Red color was used to highlight a ratio greater than 1.5. (C) Comparison of Histone H3 modification in A375 and HCT116 stable transfectants (D) A375 stable transfectants as indicated were treated with 0.03 μM chaetocin, 30μM GSK343, 10 μM UNC199 and 10 μM SGC0946 for 48 hr before measuring cell viability by SRB assay. Data are presented as relative to the WT cells without drug treatment (n=3 per group, data represent mean±SEM). (E) HCT116 stable transfectants as indicated were treated with 0.03 μM chaetocin, 30 μM GSK343, 10 μM UNC199 and 10 μM SGC0946 for 48 hr before measuring cell viability by SRB assay. Data are presented as relative to the WT cells without drug treatment (n=3 per group, data represent mean±SEM). (F) Quantification of TUNEL fluorescence intensity by flow cytometry after Dnasel treatment in A375 and HCT116 stable transfectants. (G) Expression of MTF2 and SUV39H1 in A375 and HCT-116 stable transfectants was analyze by IB. (H-I) Ezh2 and SUV39H1 were immunoprecipitated and RIP-qPCR was used to assess the associated of the MALAT-1 and NEAT-1 with each proteins.

FIGS. 18A-18B: (A) The cell viability changes in the JQ1-insensitive cells with the combined treatment of EOAI and JQ1 with a series concentrations. (B) The cell viability changes in the JQ1-sensitive cells with the combined treatment of EOAI and JQ1 with a series concentrations.

FIGS. 19A-19B: (A) A375 stable transfectants as indicated were treated with 1 μM EOAI3402143 (EOAI) for 48 hr before measuring cell viability by SRB assay. Data are presented as relative to the R-WT cells without drug treatment (n=3 per group, data represent mean±SEM). (B) HCT-116 stable transfectants as indicated were treated with 2 μM EOAI3402143 (EOAI) for 48 hr before measuring cell viability by SRB assay. Data are presented as relative to the R-WT cells without drug treatment (n=3 per group, data represent mean±SEM).

FIGS. 20A-20B: (A) A375 cells were treated with GSK343 10 μM, UNC1999 5μM without or with 0.5 μM EOAI3402143 (EOAI) for 48 hr before measuring cell viability by SRB assay. Data are presented as relative to the R-WT cells without drug treatment (n=3 per group, data represent mean±SEM). (B) HCT116 cells were treated with GSK343 10 μM, UNC1999 10 μM without or with 1 μM EOAI3402143 (EOAI) for 48 hr before measuring cell viability by SRB assay. Data are presented as relative to the R-WT cells without drug treatment (n=3 per group, data represent mean±SEM).

FIG. 21A-21C: (A) KAS seq identify peak loss in METTL3 KD A375 cells (B) GO analysis show enrichment of DNA damage related pathway (C) A375 stable transfectants as indicated were treated with 30 μM olaparib, or 10 μM rucaparib, 10 μM veliparib before measuring cell viability by SRB assay. Data are presented as relative to the WT cells without drug treatment (n=3 per group, data represent mean±SEM).

FIGS. 22A-22B: UV stress induced changes of chromatin accessibility and m⁶A methylation level. (A) Analysis of chromatin accessibility in the control wild type or Mettl3^{-/-} mESCs after UV irradiation 0 min, 2 min and 120 min. DNase I-treated TUNEL assay was performed; nucleus is counterstained by DAPI. Scale, 50 μm. (B) LC-MS/MS quantification of the m⁶A/A ratio in non-ribosomal RNA extracted from different fraction of control or Mettl3^{-/-} mESCs after UV irradiation 0 min, 2 min and 120 min; n=3 biological replicates. Error bars indicate mean±s.e.m. P values were determined by two-tailed t-test.

FIGS. 23A-23H: The knockdown of METTL3 in melanoma A375 cancer cells led to increased DNA damage and suppressed cell growth. (A) Analysis of dsDNA break in the control or METTL3 knockdown A375 cells. TUNEL assay was performed. (B) Analysis of nascent RNA synthesis in the control or METTL3 knockdown A375 cells. Nascent RNA synthesis was detected by using click-it RNA Alexa fluor 488 imaging kit. (C) Cell proliferation measured by MTS assay of control or METTL3 knockdown A375 cells. Cell numbers were normalized to the MTS signal 5 h after cell seeding. (D) Colony formation were assessed for control or METTL3 knockdown A375 cells. For panels C-D, n=3 biological replicates. Error bars indicate mean±s.e.m. P values were determined by two-tailed t-test. (E-F) m⁶A levels of caRNAs (E) and carRNAs (F) in control or METTL3 knockdown A375 cells quantified with number of reads mapped to human genome divided by reads mapped to m⁶A modified spike-in using MeRIP-seq data. (G) Repeats families (x-axis) ranked by m⁶A peak level fold-changes (y-axis) upon METTL3 knockdown versus control. For panels E-G, n=2 biological replicates. Error bars indicate mean±sd. (H) Gene Ontology (GO) enrichment analysis of genes whose upstream carRNA m⁶A level fold-changes (log2FC<−1.5) between METTL3 knockdown and control A375 cells.

FIGS. 24A-24E: The synergistic damage effects of METTL3 knockdown with DNA damage repair modulators or inhibitors of specific DNA repair pathways in cancer cells. (A-D) Analysis of cell viability in the control or METTL3 knockdown A375 cells after treatment with Bleomycin (A), 5-FU (B), AZD6738 (C) and Veliparib (D). n=3 biological replicates. Error bars indicate mean±s.e.m. P values were determined by two-tailed t-test. (E) Analysis of dsDNA break in the control or METTL3 knockdown colon cancer cells. TUNEL assay was performed.

### DETAILED DESCRIPTION

The present disclosure relates to the treatment and/or prevention of cancer. In particular, the present disclosure provides novel compositions and methods for treating and/or preventing cancer based on the attenuation of Methyltransferase-like Protein 3 (METTL3) activity in a tumor cell. In accordance with these embodiments, the present disclosure provides compositions and methods involving the use of a deubiquitinase inhibitor with or without an agent that modulates chromatin state and/or an agent that modulates DNA damage repair.

Generally, m⁶A RNA methylation plays substantial roles in regulating RNA metabolism and, in doing, so, tunes gene expression and controls biological functions. The modification is installed by the METTL3/METTL14 heterodimeric complex, and can be reversed by the two demethylases. While many studies have shown the importance of METTL3 in cancer, stem cell, and other physiology, few have shown how METTL3 itself is post-translationally regulated.

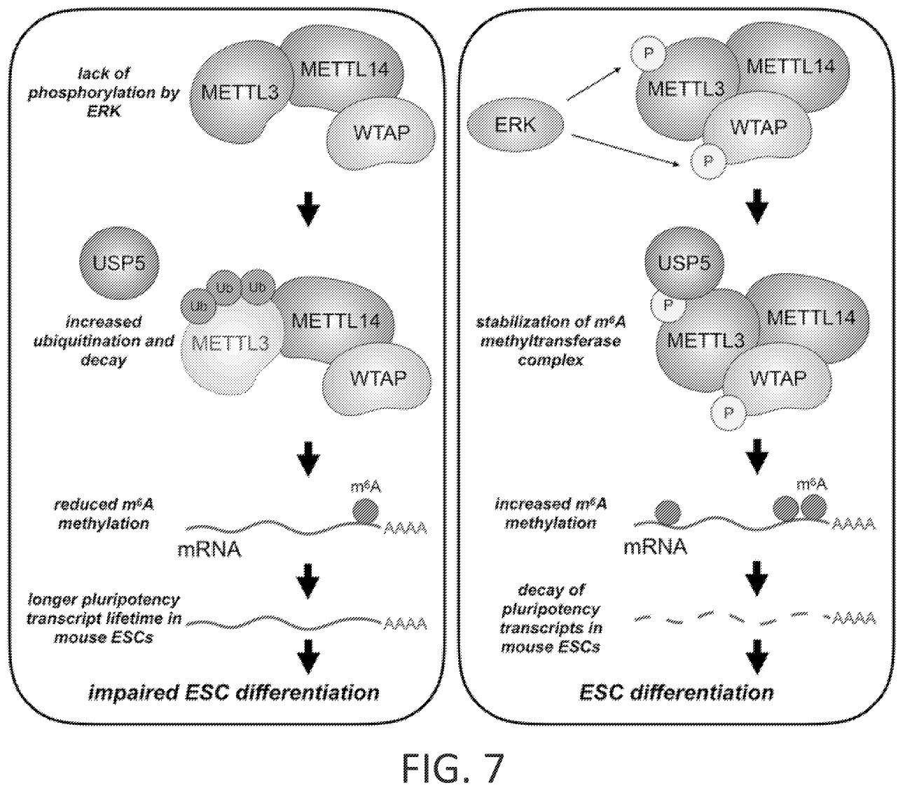

Embodiments of the present disclosure identify an ERK2-METTL3/WTAP signaling axis that regulates mESC differentiation and potentially affect tumorigenesis. Initially, a genome-wide CRISPR screen was deployed using an m⁶A methylation-dependent GFP reporter. Ras and MAPK pathway were identified as the top pathways in the positive regulation of m⁶A methylation. Biochemical studies showed that ERK proteins could phosphorylate METTL3 on S43/S50/S525 and WTAP at S306/S341. It was also found that phosphorylation of METTL3 decreases METTL3 ubiquitination through interaction with USP5. These findings explain elevated m⁶A levels on polyA-tailed RNA upon ERK activation. This pathway underlines a previously unrecognized effect of ERK activation through RNA methylation during differentiation in pluripotent mouse ESCs (see, e.g., FIG. 7).

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Coefficient of variation” (CV), also known as “relative variability,” is equal to the standard deviation of a distribution divided by its mean.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Correlated to” as used herein refers to compared to.

“Sample,” “test sample,” “specimen,” “sample from a subject,” and “patient sample” as used herein may be used interchangeably and may be a sample of blood, such as whole blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal and a human. In some embodiments, the subject may be a human or a non-human. The subject or patient may be undergoing other forms of treatment.

“Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions and Methods

Embodiments of the present disclosure pertain to the finding that ERK-mediated phosphorylation of METTL3 is important for downregulation of m⁶A-labeled pluripotency transcripts in order to induce mESC differentiation. Consistent with previous observations, m⁶A-seq data revealed extensive mRNA m⁶A methylation in mESCs. Additionally, as described further herein, upon loss of METTL3/WTAP phosphorylation, differentially methylated transcripts are enriched for genes involved in pluripotency, RNA processing, and development (similar to those found in METTL3 KO/KD studies). This supports a model in which METTL3 phosphorylation is necessary for regulation of pluripotency and differentiation.

Results provided here further explain the importance of METTL3 in regulating gene expression that leads to mESC state transitions. For example, Mettl3-deficient mESCs fail to exit pluripotency despite differentiation cues, at least in part because m⁶A destabilizes transcripts that promote pluripotency. Previous studies have shown distinct effects of Mettl3 removal between the hyper-naive state and primed naive state, the former towards promoting pluripotency and the latter differentiation. Results provided herein suggest that ERK activation may further increase m⁶A methylation on key pluripotent transcripts, thus contributing to their decay. Tuning the phosphorylation state of METTL3 may be an effective post-translational mechanism to adjust global mRNA m⁶A methylation upon signaling or stress response.

While the results provided herein indicate that phosphorylation of METTL3 affects interaction with WTAP and USP5, the effects of other signaling pathways or binding partners affected. For example, the TGF-β signaling pathway component SMAD2/3 interacts with the METTL3/METT14/WTAP complex to promote m⁶A binding to particular transcripts in mESCs. ZFP217 has also been found to interact with and sequester METTL3, thereby restricting m⁶A methylation of certain transcripts in ESCs. Knockdown of ZFP217 results in decreased lifetime of pluripotency transcripts as well. WTAP was also found to be phosphorylated by ERK. In HEK293T cells, expression of MEK, HER, or B-RAF could increase association between METTL3 and WTAP. In smooth muscle cells, IGF-1, which transmits signals along the MAPK and PI3K pathways, induces degradation of WTAP protein. This effect is mediated by the PI3K/AKT pathway, and modulation of the ERK pathway had no effect.

Genome integrity is constantly under challenge by cellular and environmental factors. DNA damage response (DDR) can detect and repair damaged DNA, and suspend cell division until the repair is complete. Studies over the last decades have emphasized the roles of chromatin components in response to DNA damage. For example, at an early stage of DDR, histone marks are installed at specific regions to make them more accessible to repair factors and to inhibit transcription from a damaged template. Despite these advances, key factors involved in DNA damage repair remain to be uncovered. Previous work has shown that m⁶A methylation is transiently induced at DNA damage sites in response to UV irradiation, and that m⁶A facilitates Pol lc recruitment to damage sites to ensure efficient DNA repair and cell survival. However, the underlying mechanism is not clear.

However, m⁶A methylation of chromosome-associated regulatory RNAs (carRNAs), in particular promoter-associated RNA (paRNA), enhancer RNA (eRNA), and repeats RNAs by METTL3 controls their stability on the chromatin. And depletion of METTL3 in mouse embryonic stem cells (mESCs) elevates levels of carRNAs and promotes open chromatin state and downstream gene transcription in mESC2. Therefore, carRNA m⁶A methylation may impact DNA damage repair.

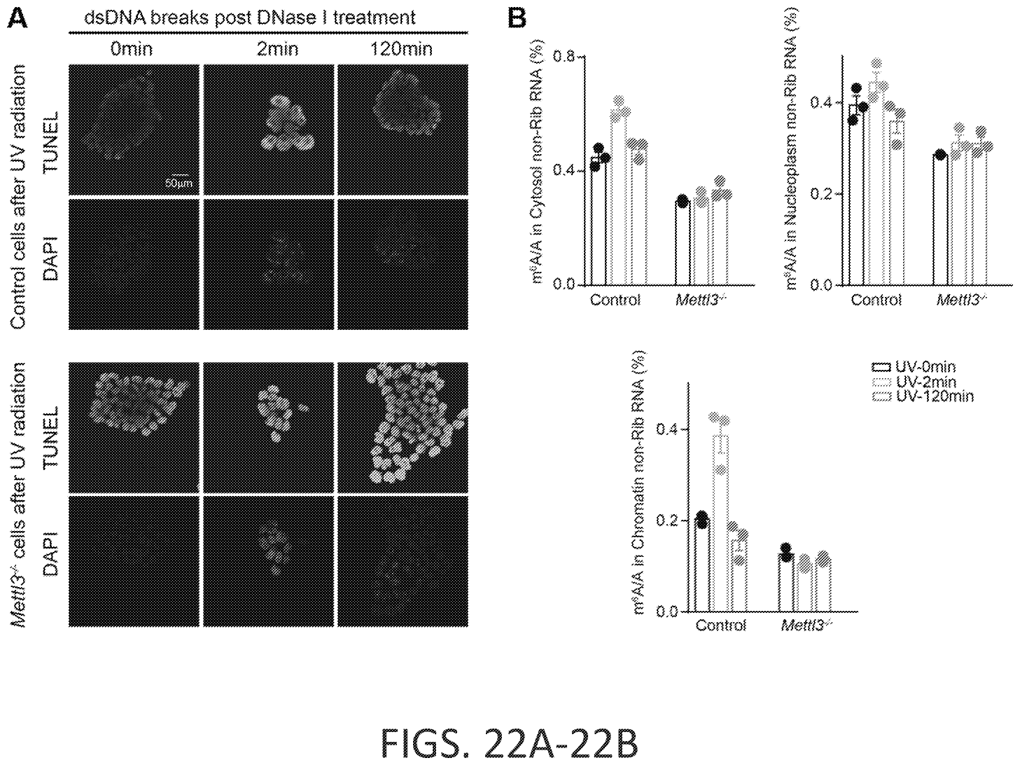

For example, a rapid increase of chromatin openness in mESCs was previously observed upon UV irradiation, followed by a reversal back to the normal level; however, in Mettl3 knockout mESCs, the increased chromatin accessibility induced by UV damage could not reverse back after 2 hours (see, e.g., FIG. 22A). The m⁶A level of RNA extracted from different cellular fractions (with depletion of ribosomal RNA) was also measured, and it was found that the m⁶A level of chromosome-associated RNA (caRNA) changed the most in response to UV stress. The level almost doubled in wildtype cells but not in Mettl3 knockout mESCs (see, e.g., FIG. 22B). Thus, upon UV-induced DNA damage, METTL3 can be recruited to methylate caRNAs (including carRNA and pre-mRNA) to regulate chromatin state and transcription, which is crucial to DNA damage repair.

The results described in the present disclosure demonstrate that METTL3 modulates dsDNA damage repair signaling (e.g., homologous recombination (HR) and non-homologous end joining (NHEJ) pathways); therefore, inhibition of METTL3 will affect tumors associated with chromosome and microsatellite instability and/or DNA damage repair defect (e.g., BRCA1/BRCA2 mutations, DNA mismatch repair mutations, p53 mutations, and the like). METTL3 modulation is thus a target for anti-cancer therapies, including but not limited to, therapies designed to target METTL3 directly, indirectly (e.g., USP5 modulation), and/or in combination with other agents that modulate chromatin state or DNA damage repair.

In accordance with these embodiments, the compositions and methods provided herein can target major repair pathways and key proteins used to process the various types of DNA damage. In non-homologous end-joining (NHEJ), for example, the Ku70/Ku80 complex binds to the DNA double-strand break ends and recruits the other indicated components. In base-excision repair (BER), poly(ADP-ribose) polymerase-1 (PARP-1) detects and binds to single-strand breaks and ensures accumulation of other repair factors at the breaks. Single-strand breaks containing modified DNA ends are recognized by damage-specific proteins such as apurinic/apyrimidinic endonuclease (APE1), which subsequently recruits Polβ and XRCC1-DNA ligase Ma to accomplish the repair. The proteins involved in these pathways have been shown to be dysregulated in various types of cancers, and METTL3 inhibition can be used to target one or more of them to treat and/or prevent cancer; these targets include, but are not limited to, PARP-1, APE1, XRCC1, DNA ligase III, Ku70/Ku80, DNA-PK, Artemis, XRCC4, DNA ligase IV, XLF, RPA, BRCA1, BRCA2, PALB2, and RAD51, among others.

For example, and with regard to NHEJ specifically, inhibitors of DNA-PK, including NU7026 and NU7441, were found to induce extreme sensitivity to ionizing radiation as well as DNA-damaging agents in preclinical studies. The dual mTOR and DNA-PKcs inhibitor CC-115 is undergoing early clinical evaluation. KU-0060648 is a potent dual inhibitor of DNA-PK and PI-3K, which has recently been reported to enhance etoposide and doxorubicin.

In another example, inactivation of DNA damage response proteins is also observed in various cancers. The p53 gene is one of the most frequently mutated genes in human sporadic cancers. Although the reported frequencies of p53 mutations vary among the types of cancer, it is estimated that more than half of cancers might have inactivated p53 due to mutations, deletions, loss of heterozygosity of the gene, or decreased expression. Although inactivating mutations in ATM, BRCA1, or BRCA2 are less frequent than those in the p53 gene, decreased expression of ATM, the MRN complex, Chk2, RAD51, BRCA1, BRCA2, and ERCC1 is frequently observed. Promoter hypermethylation of the BRCA1 gene has also been observed and may be one of the predominant mechanisms for deregulation of the BRCA1 gene.

In another example, ATM and the MRN complex, which act as sensors or mediators in the DNA damage response, have been considered to be targets for cancer therapy, and several promising ATM inhibitors have been developed. KU55933, for example, is the first specific inhibitor of ATM, and it inhibits radiation-induced ATM-dependent phosphorylation events and sensitizes cancer cells to radiation and topoisomerase inhibitors. KU60019, an improved analog of KU55933, inhibits the DNA damage response and effectively radiosensitizes human glioma cells. Mirin is an inhibitor of the MRN complex, which prevents MRN-dependent activation of ATM without affecting ATM protein kinase activity and inhibits MRE11-associated exonuclease activity. Telomelysin is another inhibitor that inhibits the MRN complex through the adenoviral E1B-55 kDa protein. Additionally, schisandrin B was recently identified as a moderate selective ATR inhibitor (may also affect ATM at high concentrations). Recently, two novel ATR inhibitors, NU6027 and VE-821, were also shown to sensitize cells to a variety of DNA-damaging agents in preclinical studies.

Taken together, and as described further herein, mechanisms related to METTL3 inhibition can be used alone or in combination with the various modulators targeting DNA repair pathways and key proteins used to process the various types of DNA damage. Thus, embodiments of the present disclosure include a composition for attenuating METTL3 in a tumor cell. In accordance with these embodiments, the composition includes at least one deubiquitinase inhibitor, at least one chromatin state modulator and/or at least one DNA damage modulator, and a pharmaceutically acceptable carrier or excipient.

In some embodiments, the at least one deubiquitinase inhibitor targets Ubiquitin Carboxyl-terminal Hydrolase 5 (USP5). In some embodiments, the at least one chromatin state modulator includes a bromodomain and extraterminal domain (BET) inhibitor, a histone methyl transferase (HMT) inhibitor, and/or a poly(ADP-ribose) polymerase 1 (PARP1) inhibitor. In some embodiments, the at least one DNA damage repair modulator induces DNA damage and/or inhibits DNA repair. In some embodiments, inhibiting USP5 attenuates 1METTL3 protein stability and/or activity.

In some embodiments, the at least one deubiquitinase inhibitor comprises EOAI3402143, vialinin, WP1130, mebendazole, PYR-41, gossypetin, formonectin, suramin, and/or any combinations thereof. In some embodiments, the BET inhibitor comprises a thienotriazolodiazepine, OTX015, BET-d246, ABBV-075, I-BET 151, I-BET 762, CPI 203, PFI-1, RVX-208, Dinaciclib, and/or any combinations thereof. In some embodiments, the thienotriazolodiazepine is JQ1.

In some embodiments, the HMT inhibitor comprises chaetocin, GSK343, UNC199, SGC0946, F5446, Pinometostat, EPZ004777, EPZ005687, tazemestostat, JQEZ5, CPI-1205, EPZ001989, EBI-2511, PF-06726304, El1, GSK503, GSK126, CPI-169, ZLD 1039, SAH-EZH2, NSC 617989, CPI-169, CPI-360, EPZ6438, and/or any combinations thereof.

In some embodiments, the PARP inhibitor comprises olaparib, rucaparib, veliparib, talazoprib, AG-14361, INO-1001, A-966492, PJ34, Niraparib, UPF 1069, ME0328, Pamiparib, NMS-P118, E7449, picolinamide, Benzamide, Nu1025, Iniparib, AZD2461, BGP-15, and/or any combinations thereof.

In some embodiments, the at least one DNA damage repair modulator comprises bleomycin, 5-FU, ceralasertib (AZD6738), cisplatin, oxaliplatin, carboplatin, Cytoxan, and/or any combinations thereof.

In some embodiments, the compositions of the present disclosure comprise at least one deubiquitinase inhibitor and a BET inhibitor. In some embodiments, the composition comprises at least one deubiquitinase inhibitor and an HMT inhibitor. In some embodiments, the composition comprises at least one deubiquitinase inhibitor and a PARP inhibitor. In some embodiments, the composition comprises at least one deubiquitinase inhibitor and at least one DNA damage repair modulator.

Embodiments of the present disclosure also include a method of treating or preventing cancer in a subject. In accordance with these embodiments, the method includes administering any of the pharmaceutical compositions described herein to the subject. In some embodiments, a method of treating or preventing cancer in a subject includes administering a composition comprising at least one deubiquitinase inhibitor, and at least one of a bromodomain and extraterminal domain (BET) inhibitor, a histone methyl transferase (HMT) inhibitor, a poly(ADP-ribose) polymerase 1 (PARP1) inhibitor, and/or a DNA damage repair modulator. In some embodiments of the method, the composition further comprises a pharmaceutically acceptable carrier or excipient.

In some embodiments, the composition is administered to a subject diagnosed with cancer in order to treat the cancer. In some embodiments of the method, the at least one deubiquitinase inhibitor comprises EOAI3402143, vialinin, WP1130, mebendazole, PYR-41, gossypetin, formonectin, suramin, and/or any combinations thereof. In some embodiments of the method, the BET inhibitor comprises a thienotriazolodiazepine, OTX015, BET-d246, ABBV-075, I-BET 151, I-BET 762, CPI 203, PFI-1, RVX-208, Dinaciclib, and/or any combinations thereof. In some embodiments of the method, the HMT inhibitor comprises chaetocin, GSK343, UNC199, SGC0946, F5446, Pinometostat, EPZ004777, EPZ005687, tazemestostat, JQE,Z5, CPI-1205, EPZ001989, EBI-2511, PF-06726304, El1, GSK503, GSK126, CPI-169, ZLD 1039, SAH-EZI-12, NSC 617989, CPI-169, CPI-360, EPZ6438, and/or any combinations thereof. In some embodiments of the method, the PARP inhibitor comprises olaparib, rucaparib, veliparib, talazoprib, AG-14361, INO-1001, A-966492, PJ34, Niraparib, UPF 1069, ME0328, Pamiparib, NMS-P118, E7449, picolinamide, Benzamide, Nu1025, Iniparib, AZD2461, BGP-15, and/or any combinations thereof.

In some embodiments of the method, the composition attenuates METTL3 stability and/or activity and induces apoptosis of a cancer cell. In some embodiments of the method, the composition comprises at least one deubiquitinase inhibitor and at least one BET inhibitor. In some embodiments of the method, the combination of the at least one deubiquitinase inhibitor and the at least one BET inhibitor exhibits a synergistic effect on cancer cell viability.

In some embodiments of the method, the composition comprises at least one deubiquitinase inhibitor and at least one HMT inhibitor. In some embodiments of the method, the combination of the at least one deubiquitinase inhibitor and the at least one HMT inhibitor exhibits a synergistic effect on cancer cell viability. In some embodiments of the method, the composition comprises at least one deubiquitinase inhibitor and at least one PARP inhibitor. In some embodiments of the method, the combination of the at least one deubiquitinase inhibitor and the at least one PARP inhibitor exhibits a synergistic effect on cancer cell viability. In some embodiments of the method, the combination of the at least one deubiquitinase inhibitor and the at least one DNA damage repair modulator or inhibitor that blocks DNA damage repair exhibits a synergistic effect on cancer cell viability.

In some embodiments of the method, the cancer is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, glioblastoma, lymphoma, and leukemia.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

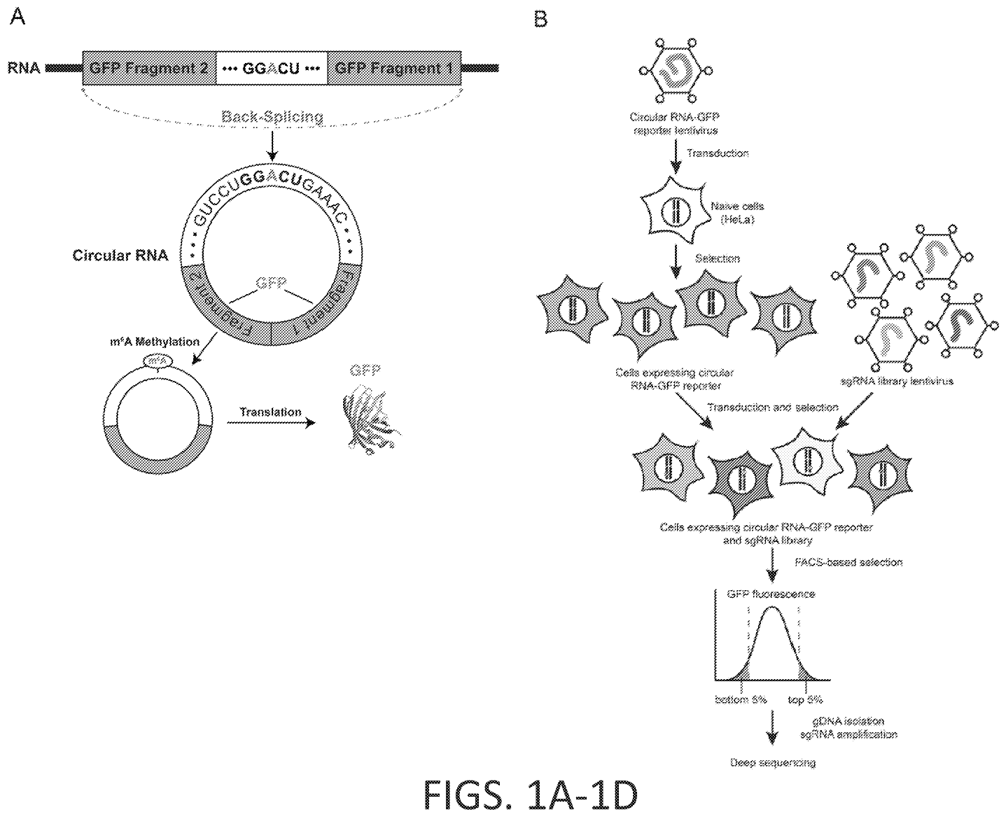

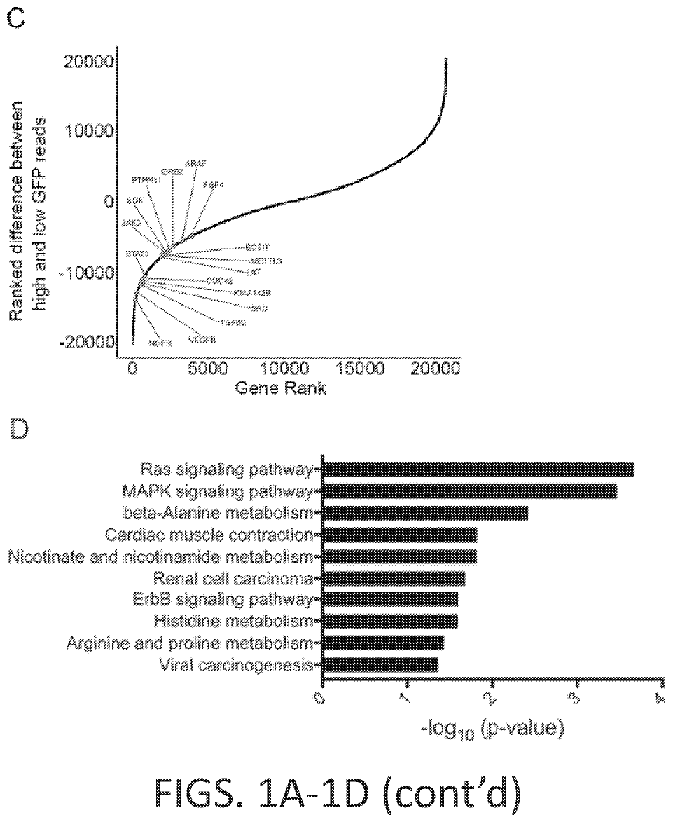

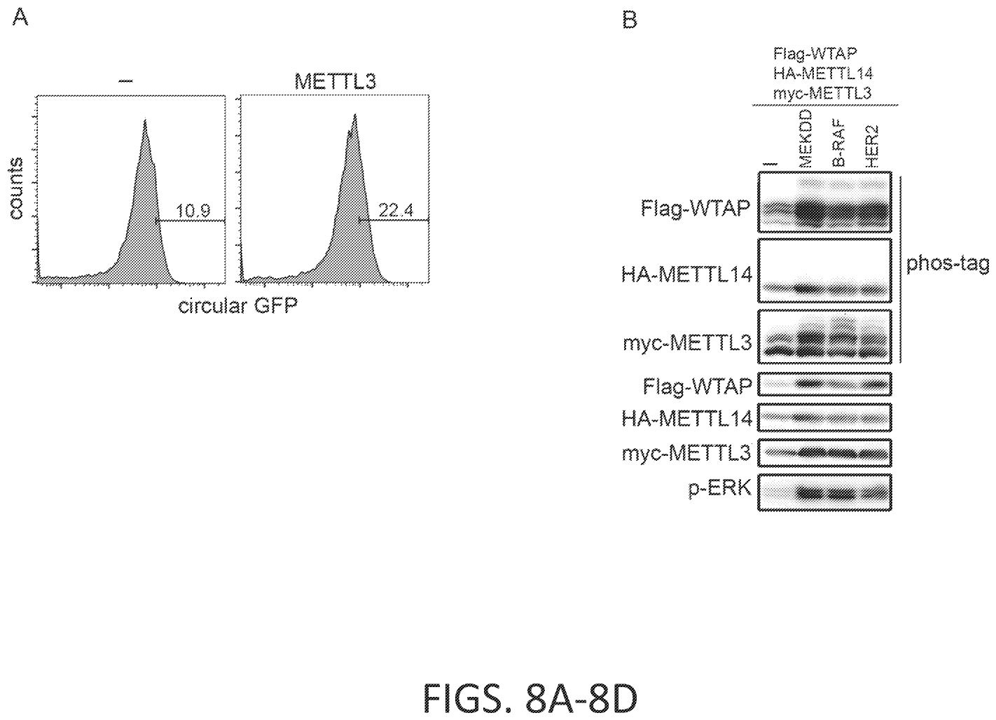

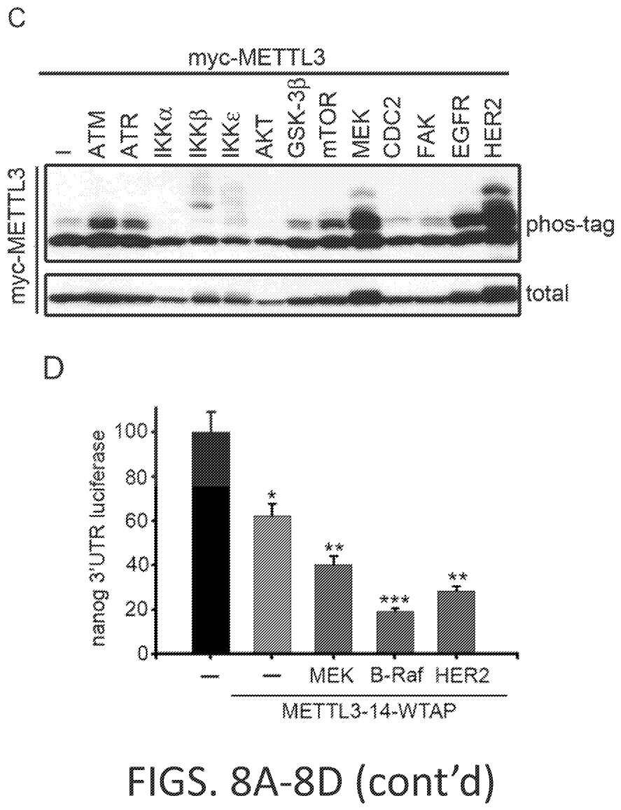

ERK Activation Promotes mRNA m⁶A Methylation. To identify new regulators of m⁶A RNA methylation, a circular RNA GFP reporter was employed containing a GGACU motif in HeLa cells. The GFP pre-mRNA transcript was assembled by back-splicing to generate a circular RNA that joins two exon fragments of GFP, as depicted in FIG. 1A. m⁶A methylation of the GGACU motifs on the circular RNA can drive translation initiation of the GFP transcript, producing GFP fluorescence signal. Consequently, the GFP signal from this circular RNA reporter can be used as readout of m⁶A methylation. Consistent with a previous report, overexpression of METTL3 increased GFP expression (FIG. 8A). Next, a CRISPR knockout-based genomic screen was performed targeting 19,050 genes and 1,864 miRNA. Combining a CRISPR knockout library with a circular RNA m⁶A-GFP reporter allowed screening for possible regulators of m⁶A methylation (FIG. 8B). Knockout of genes that promote or suppress m⁶A methylation would decrease or increase translation of the GFP transcript, respectively. Cells with the top and bottom 5% of GFP expression were therefore collected, followed by high-throughput sequencing in order to identify negative and positive regulators of m⁶A methylation, respectively. The genes that were enriched in the low-GFP-expressing and the high-GFP-expression populations were compared (these data can be made available upon request). Knockout of METTL3 led to low GFP signal in the screen (FIG. 1C). Pathway enrichment analysis of the gRNAs in low-GFP-expressing cells identified genes involved in the Ras and MAPK signaling pathways (FIG. 1D), such as FGF4, EGF, ARAF, GRB2, and PTPN11 (FIG. 1D and Table 1).

TABLE 1
sgRNA Identified Related to Ras and MAPK Pathways (see FIG. 1).

| Gene Name | lowGFP | highGFP | numSGRNA | rankLowGFP | rankHighGFP | rank |
|---|---|---|---|---|---|---|
| INS | 313.25 | 114.5 | 4 | 3429.5 | 18714 | 73 |
| STK4 | 308.2 | 127.2 | 5 | 3675.5 | 17938 | 130 |
| NGFR | 272.6 | 95.2 | 5 | 5731 | 19564 | 167 |
| MAPK10 | 276.333333 | 110.333333 | 6 | 5465.5 | 18909 | 208 |
| DUSP10 | 371.4 | 167.4 | 5 | 1559.5 | 14734 | 237 |
| RASA3 | 270.666667 | 113.5 | 6 | 5872 | 18768.5 | 269 |
| RPS6KA2 | 256.6 | 95 | 5 | 6894.5 | 19572 | 298 |
| MECOM | 389.4 | 185 | 5 | 1193.5 | 13082 | 430 |
| RAC2 | 274.833333 | 136.5 | 6 | 5585 | 17254 | 477 |
| TGFB2 | 259.833333 | 122.5 | 6 | 6651.5 | 18270.5 | 490 |
| ETS1 | 294.2 | 152.6 | 5 | 4393.5 | 15992.5 | 495 |
| HSPA1B | 253.4 | 114.2 | 5 | 7140 | 18733 | 496 |
| BAD | 292.5 | 152 | 6 | 4495.5 | 16037.5 | 504 |
| RALA | 295.25 | 154.75 | 4 | 4336.5 | 15822 | 511 |
| MKNK2 | 299.166667 | 158.166667 | 6 | 4116 | 15511.5 | 531 |
| MAPT | 239.333333 | 99.6666667 | 3 | 8234.5 | 19395.5 | 579 |
| MAP3K8 | 255.25 | 124.75 | 4 | 6995 | 18117.5 | 585 |
| EXOC2 | 272.6 | 142.2 | 5 | 5731 | 16831 | 598 |
| SHC1 | 279.6 | 152.2 | 5 | 5247.5 | 16024.5 | 683 |
| CDC42 | 241 | 111.166667 | 6 | 8112.5 | 18859.5 | 693 |
| CACNB1 | 280 | 155.833333 | 6 | 5220.5 | 15721.5 | 751 |
| RPS6KA1 | 569 | 211.4 | 5 | 127 | 10612 | 761 |
| GAB1 | 252 | 136.333333 | 6 | 7228.5 | 17267.5 | 907 |
| MET | 309.833333 | 180.5 | 6 | 3607 | 13512 | 941 |
| PRKACB | 296.5 | 175.666667 | 6 | 4269 | 13957 | 1018 |
| ELK4 | 327.833333 | 197 | 6 | 2822 | 11958 | 1222 |
| BRAP | 250 | 151 | 5 | 7382 | 16130.5 | 1372 |
| MAP3K1 | 301.5 | 189.833333 | 6 | 3991 | 12608 | 1429 |
| MAP2K3 | 241.833333 | 151.666667 | 6 | 8056 | 16062.5 | 1723 |
| ECSIT | 447.833333 | 241.166667 | 6 | 517 | 7917.5 | 2033 |
| EGF | 224.666667 | 145.5 | 6 | 9519.5 | 16583.5 | 2231 |
| GRIN2B | 321.5 | 232.333333 | 6 | 3083 | 8694 | 3197 |
| ARAF | 213.666667 | 152.666667 | 6 | 10454 | 15987.5 | 3258 |
| CALM3 | 239.833333 | 178.666667 | 6 | 8199 | 13686.5 | 3300 |
| ELK1 | 160.333333 | 76.3333333 | 6 | 15337 | 20124 | 3900.5 |
| MAPK8IP2 | 248.8 | 194.2 | 5 | 7490.5 | 12212.5 | 3965 |
| MEF2C | 133 | 19.2 | 5 | 17501 | 20636 | 5608.5 |
| INS-IGF2 | 328.666667 | 274.833333 | 6 | 2789 | 5347.5 | 6308 |
| RASA1 | 133 | 84.8333333 | 6 | 17501 | 19924.5 | 6485 |
| DUSP7 | 213.166667 | 188.166667 | 6 | 10503.5 | 12783.5 | 6663 |
| RAB5C | 205.166667 | 181.833333 | 6 | 11287 | 13397.5 | 6888 |
| PLA2G1B | 242.2 | 220.6 | 5 | 8023 | 9728.5 | 7421 |
| RASSF5 | 311.4 | 280.4 | 5 | 3527.5 | 4992.5 | 7756 |
| CACNG3 | 216.166667 | 202.166667 | 6 | 10238 | 11459.5 | 8114.5 |
| PAK3 | 286.6 | 267 | 5 | 4807.5 | 5897.5 | 8347 |
| EFNA4 | 213.8 | 204.8 | 5 | 10442.5 | 11226 | 8863 |
| TGFA | 146.2 | 138.8 | 5 | 16487 | 17089 | 9164 |
| NLK | 170.166667 | 163.166667 | 6 | 14487.5 | 15084.5 | 9178 |
| CACNB2 | 409.333333 | 372 | 6 | 872.5 | 1438.5 | 9243.5 |
| FLT1 | 247.833333 | 239.333333 | 6 | 7557 | 8079.5 | 9310.5 |
| RASA4 | 495 | 422 | 3 | 271 | 730.5 | 9426.5 |
| CACNA1C | 211.8 | 212 | 5 | 10626.5 | 10562 | 10519 |
| HTR7 | 252.166667 | 256.166667 | 6 | 7218.5 | 6694 | 11385.5 |
| MAP2K5 | 217.166667 | 246 | 6 | 10141.5 | 7495.5 | 14554 |
| GNG2 | 201.833333 | 232 | 6 | 11599 | 8728.5 | 14808 |
| SOS2 | 253.166667 | 324.5 | 6 | 7155.5 | 2759 | 16387 |
| PLCG2 | 234.8 | 314.2 | 5 | 8640 | 3184 | 17306 |

To determine how the RAS/MAPK pathway could alter m⁶A methylation, the status of the m⁶A methyltransferase complex during MAPK pathway activation was investigated. A phos-tag gel revealed that constitutively active MEK S218D/S222D, B-RAF V600E, or HER2 V659E, increased the phosphorylation-dependent mobility shift of METTL3 and WTAP, but not METTL14 (FIG. 8B). A panel of 13 oncogenic kinases, including ATM, ATR, IKK-α, IKK-β, IKK-ε, AKT, GSK-3β, mTOR, MEK, CDC2, FAK, EGFR, and HER2 was co-transfected with METTL3 in 293T cells. As shown in FIG. 8C, MEK and HER2, which activate ERK, induced the most significant phosphorylation-dependent mobility shift of METTL3. Nanog 3′ UTR, which contains three m⁶A consensus RRACU motif sites that mediate the methylation-dependent decay of NANOG, was also used as a readout of the cellular m⁶A methylation activity. Consistently, ERK activation promotes NANOG destabilization (FIG. 8D), presumably by increasing methylation of its 3′ UTR. Together, these results show that the activation of MAPK pathway promotes mRNA m⁶A methylation.

### Example 2

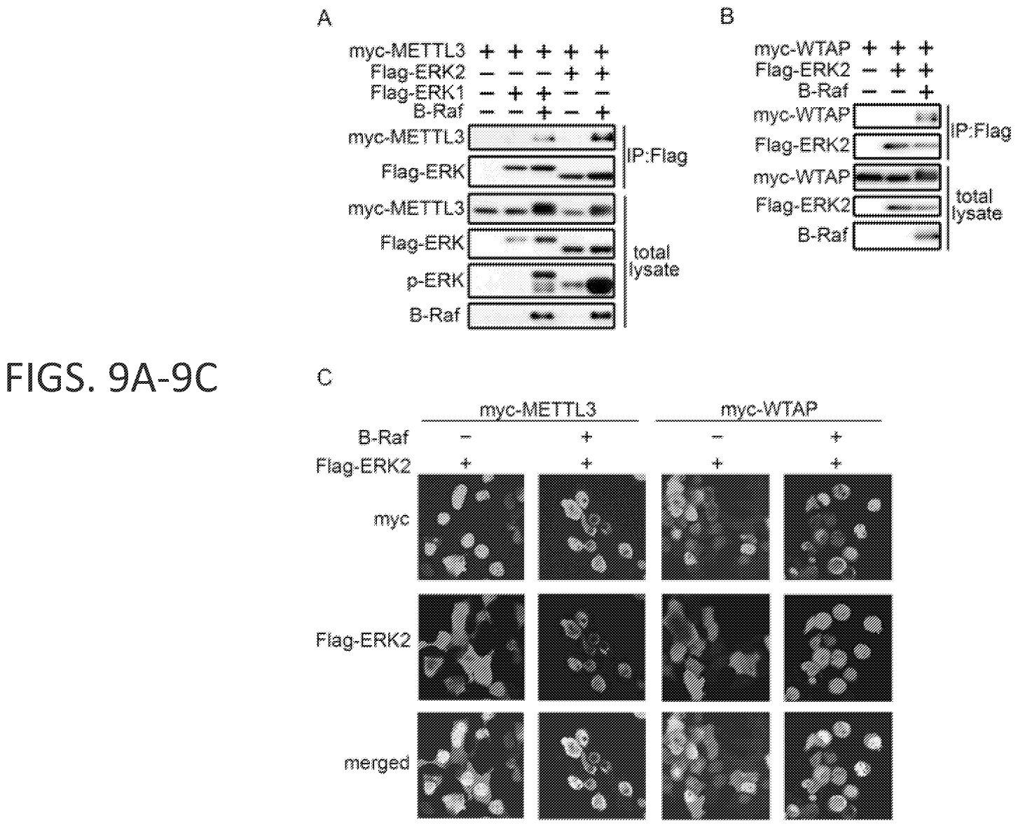

ERK Phosphorylates METTL3 and WTAP. To determine how ERK activates m⁶A methylation, it was first tested whether ERK interacts with and phosphorylates the mRNA m⁶A methyltransferase complex. Co-immunoprecipitation showed that METTL3 associates with ERK1 and ERK2 upon B-RAF transfection (FIG. 9A). Considering that ERK1 and ERK2 are highly similar and possess identical substrate specificity in vitro, the analysis focused on ERK2 hereafter because ERK2 expression exceeds ERK1 in most cells. The interaction between ERK2 and WTAP was also observed after Raf activation (FIG. 9B). After MEK stimulation, activated ERK translocates into cell nuclei to activate nuclear substrates, or forms a dimer to activate cytoplasmic substrates. As shown in FIG. 9C, ERK activated by B-RAF co-localizes with METTL3 and WTAP in the nucleus, suggesting that METTL3 complex could be a nuclear substrate of ERK.

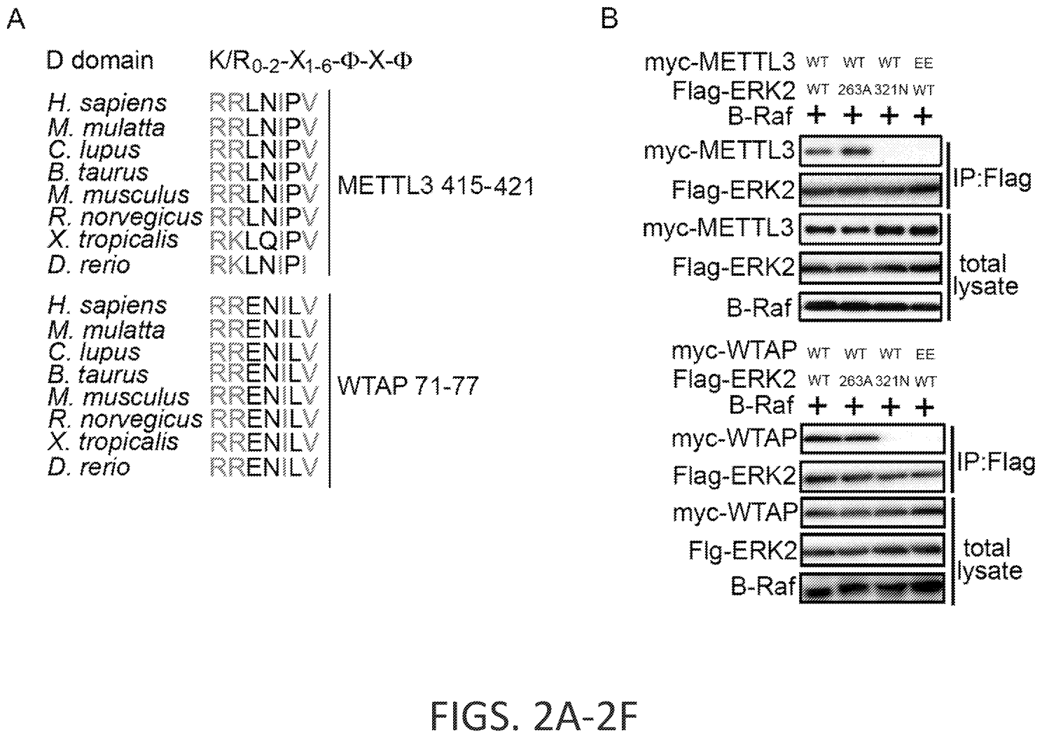

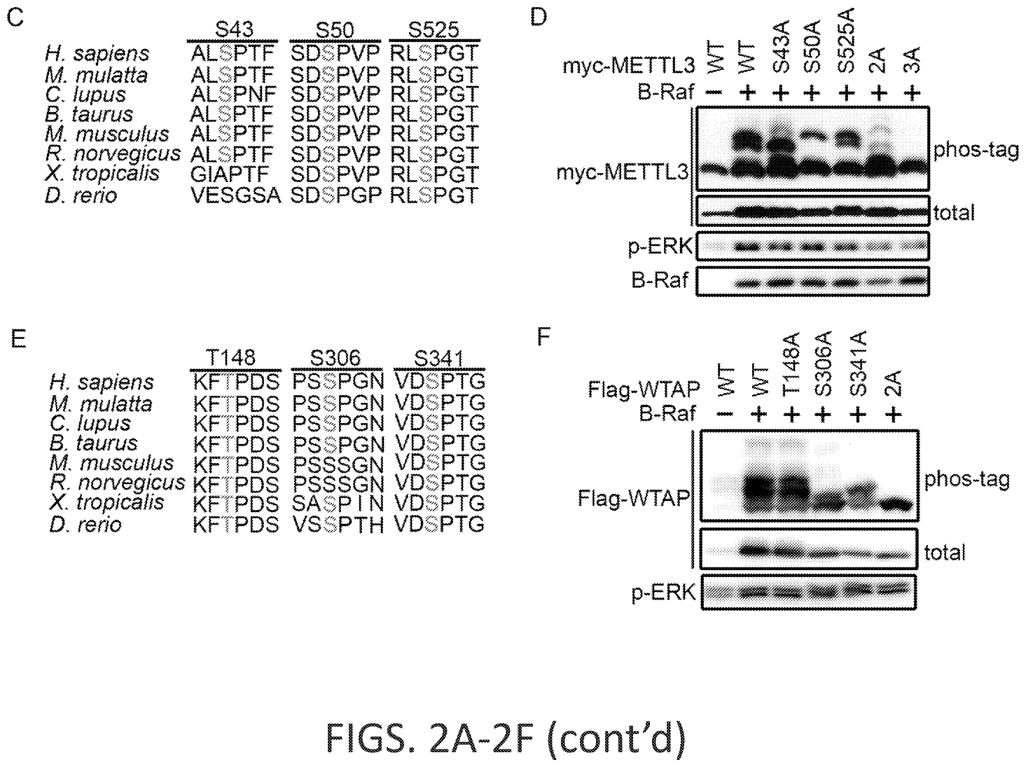

ERK displays a specificity for phosphorylation at the serine/threonine-proline (S/T-P) motif. Since the S/T-P motif is found in many proteins, ERK either uses a common docking domain (CD) to bind to a D domain (K/R₀₋₂-X₁₋₆-φ-X-φ) or uses the F-site recruitment site (FRS) to bind to the F-site (FX-F/Y-P). Analysis using the Eukaryotic Linear Motif database (http://elm.eu.org) revealed residues 415-421 in METTL3 and residues 71-77 in WTAP as potentially conserved D domains (FIG. 2A). It was found that a CD mutant (321N) form of ERK2, but not an FRS mutant (263A) form, abolished its interaction with METTL3 and WTAP (FIG. 2B). Mutational analysis of the putative D domain residues of METTL3 and WTAP also abolished the interaction, further showing direct interaction of ERK with METTL3 and WTAP.

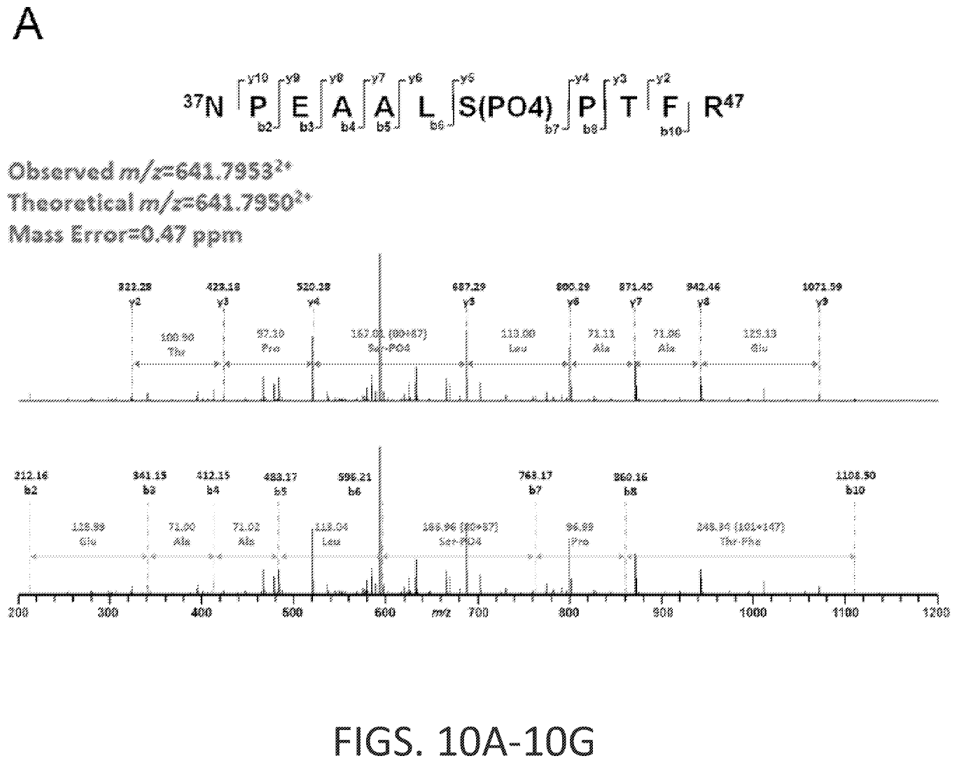

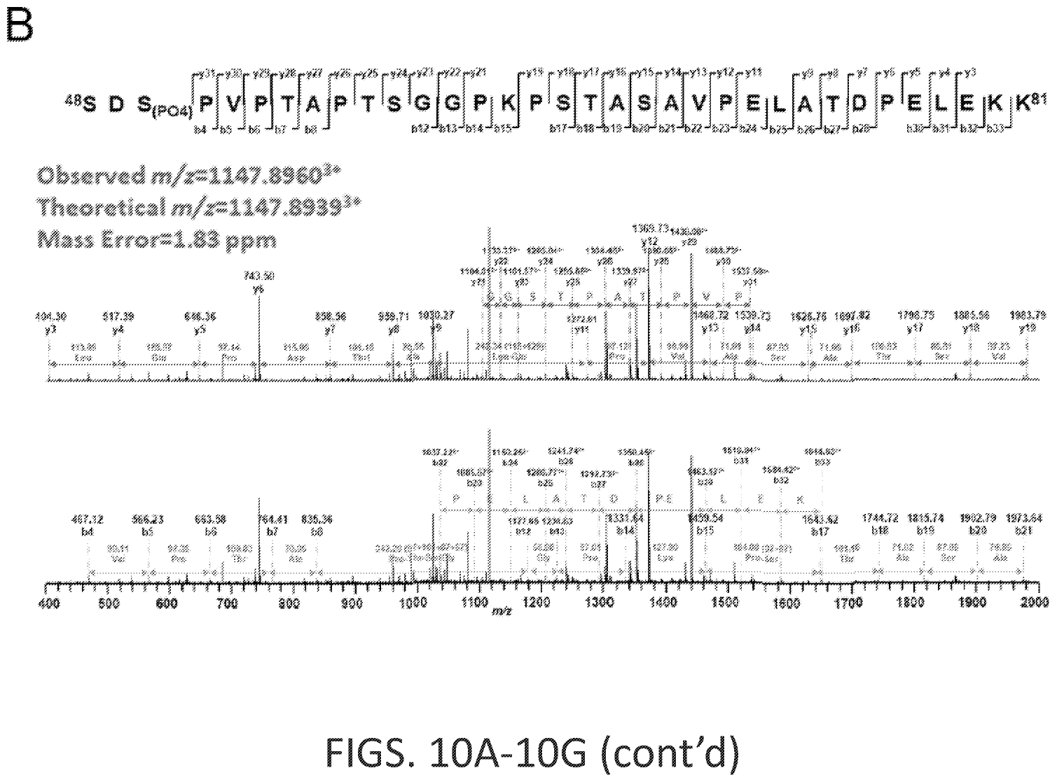

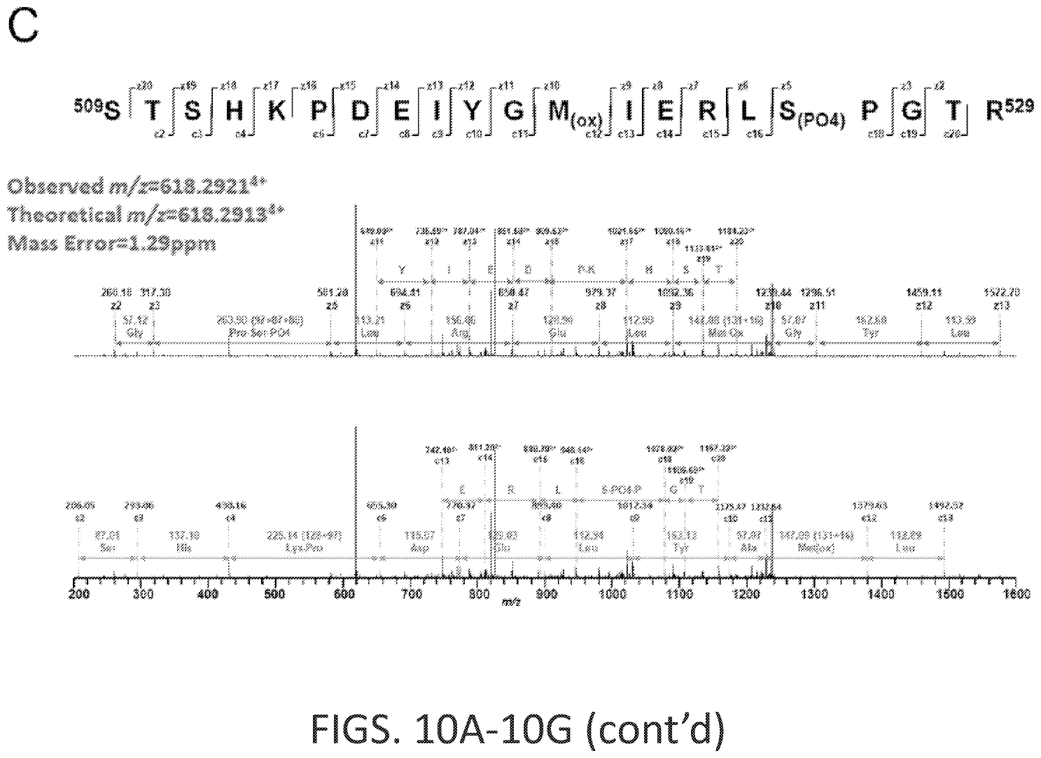

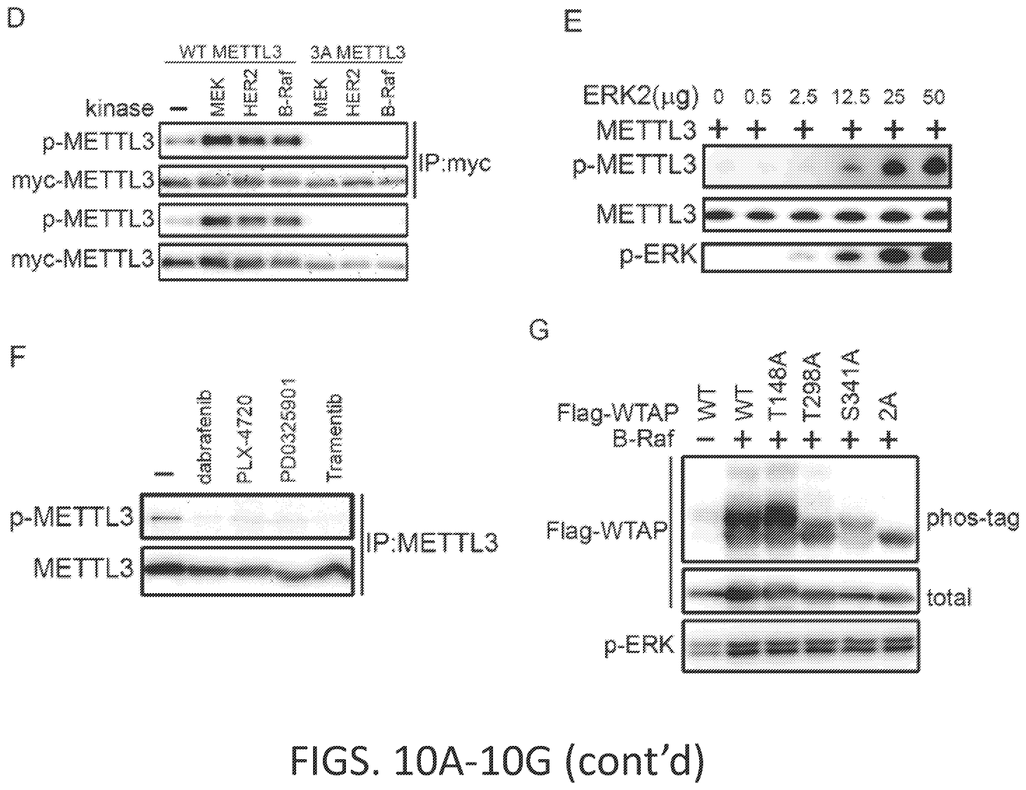

Given the physical interaction between ERK and METTL3, and the phosphorylation- based mobility shift induced by the ERK activation, experiments were conducted to identify phosphorylation sites on METTL3. Mass spectrometry analysis showed that ERK phosphorylates METTL3 at three highly conserved residues S43, S50, and S525 (FIG. 2C and FIGS. 10A-10C). Mutational analysis further confirmed these three sites as main ERK phosphorylation sites (FIG. 2D).

To investigate METTL3 phosphorylation by ERK inside cells, a polyclonal antibody was generated that targets S43-phosphorylated METTL3. This antibody recognizes S43-phosphorylated METTL3 but not a mutant form of METTL3, METTL3 3A, with all three phosphorylation serine sites replaced with alanine (FIG. 10D). This P-S43 antibody was then used as a tool to monitor METTL3 phosphorylation. An in vitro kinase assay demonstrated that METTL3 was phosphorylated by activated ERK2, and the phosphorylated form could be detected by the anti-p-METTL3 (S43) antibody (FIG. 10E). Furthermore, the p-METTL3 (S43) in A375 cells, a human melanoma cell line with constitutively active ERK due to a B-RAF V600E mutation, was abrogated by treatments with B-RAF inhibitors (dabrafenib and PLX4720) or MEK inhibitors (PD0325901 and trametinib) (FIG. 10F), supporting that S43 phosphorylation on METTL3 is installed through the ERK pathway.

To determine the phosphorylation sites of WTAP, experiments were conducted to determine whether mutations of the S/T-P motif affect the ERK-induced phosphorylation. Among the three S/T-P motifs in human WTAP (FIG. 2E), it was found that S306 and S341 are the main ERK phosphorylation sites of human WTAP (FIG. 2F). It was also observed that S306 is not conserved in mouse and rat WTAP orthologs; however, there is a unique S/T-P motif at T298 in mouse and rat WTAP, which can also be phosphorylated by ERK (FIG. 10G). In conclusion, these results show that ERK interacts with and phosphorylates METTL3 and WTAP.

### Example 3

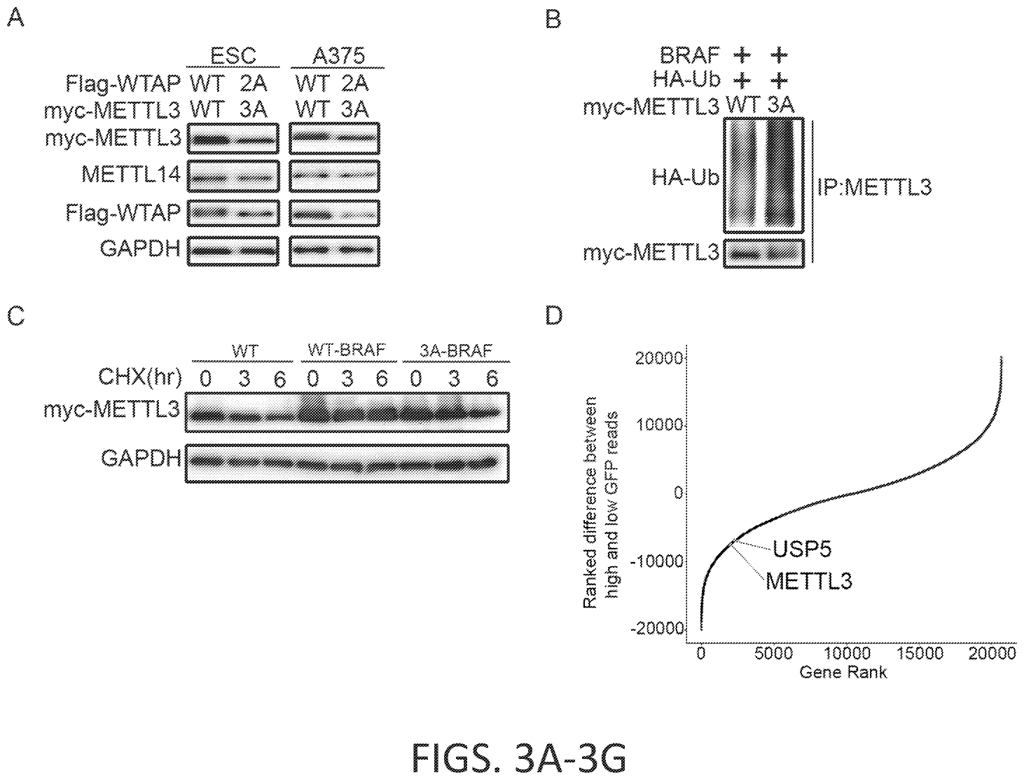

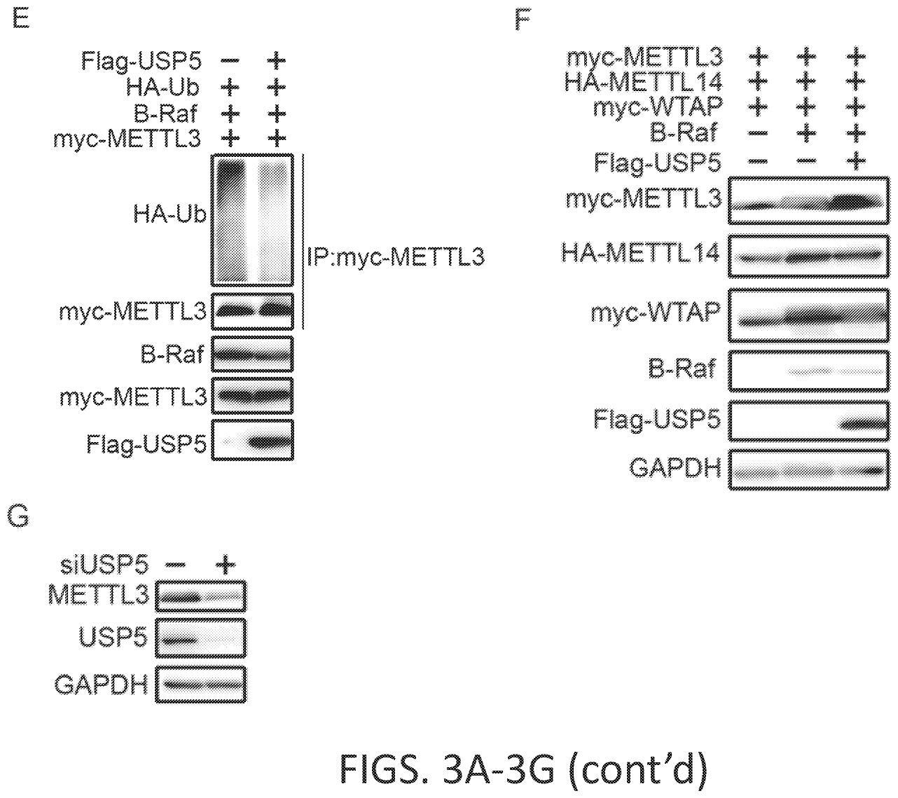

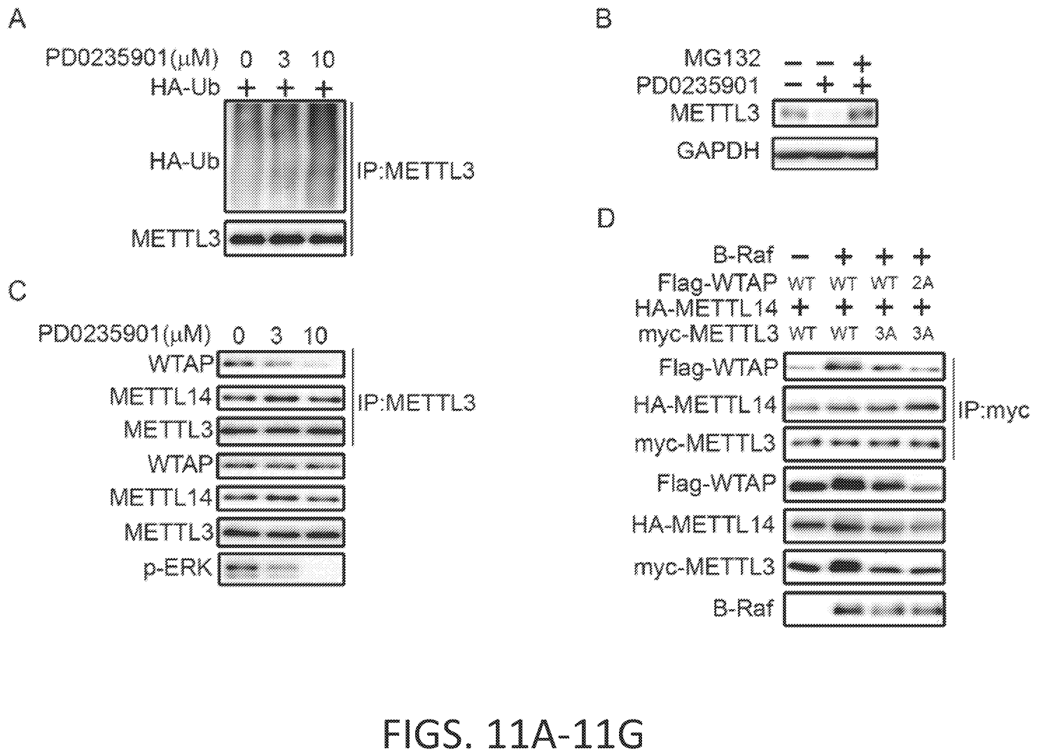

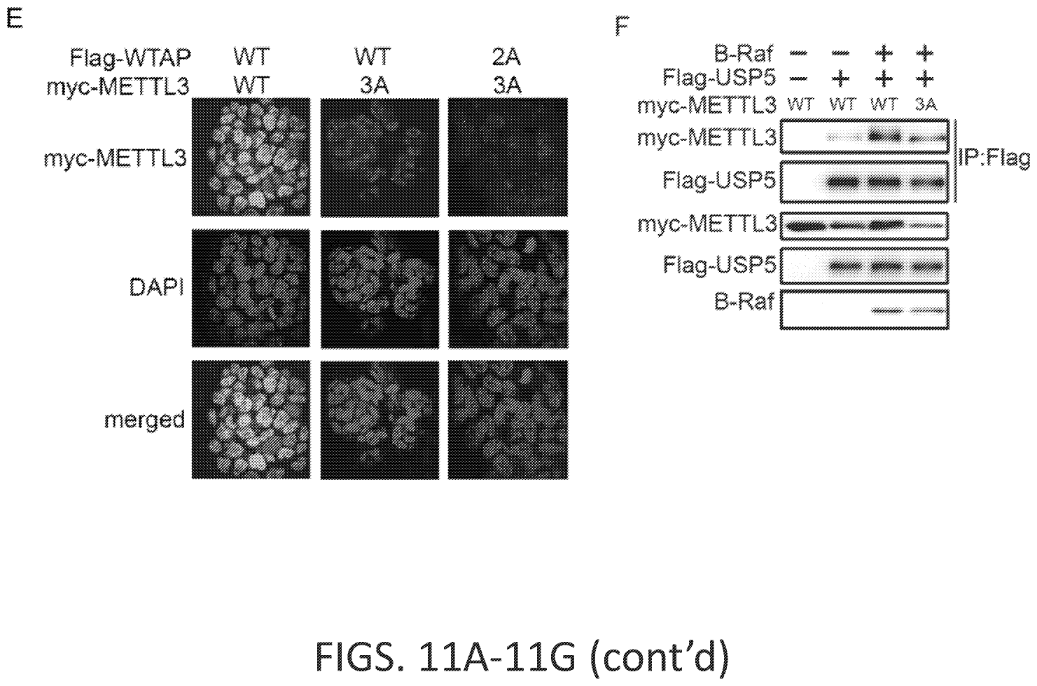

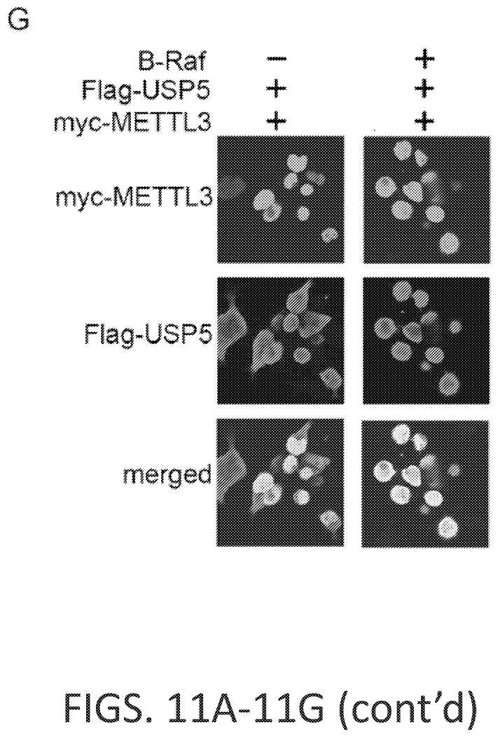

USP5 is Required for ERK-Mediated METTL3 Stabilization. Next, experiments were conducted to investigate how ERK-induced phosphorylation increases RNA m⁶A methyltransferase complex activity. It was observed that ERK activation increased the wild-type (WT) but not 3A METTL3 expression (FIG. 2D), and that WT METTL3 stable transfectants consistently expressed at higher levels than those of 3A METTL3 in both mouse ESCs (mESCs) and human A375 cells. (FIG. 3A). This observation suggested a model that METTL3 phosphorylation by ERK stabilizes the protein, which could explain the higher METTL3 protein level and elevated m⁶A methylation activity observed with ERK activation. Experiments were then conducted to investigate whether ERK activation could affect METTL3 stability. Inhibition of ERK by PD0325901 increased the ubiquitination (FIG. 11A) and degradation of METTL3, which was restored by addition of a proteasome inhibitor MG132 (FIG. 11B). The ubiquitination level of METTL3 3A was also higher than that of WT METTL3 (FIG. 3B). To assess more directly the effects of ERK on METTL3 stability, cycloheximide was used to suppress new protein synthesis and the degradation of METTL3 protein was monitored. As shown in FIG. 3C, ERK activation increased the stability of WT compared to that of the phosphorylation-resistant METTL3 3A.

Since METTL14 is known to stabilize METTL3, experiments were conducted to investigate whether phosphorylation of METTL3 by ERK affects the METTL3-METTL14 complex formation. The interaction between METTL3 and METTL14 was not obviously affected by ERK inhibition (FIG. 11C). Moreover, METTL3 3A also interacts with METTL14 normally (FIG. 11D). Interestingly, it was observed that ERK activation increased the interaction between METTL3 and WTAP, which became weaker with METTL3 3A and was further attenuated with non-phosphorylatable WTAP S306A/S341A (2A) (FIG. 11D). It has been shown that WTAP depletion does not affect METTL3 expression, but rather reduces the RNA binding ability of METTL3. Unbound METTL3 could readily get washed out during the preparation of immunostaining samples and lead to reduced staining density of METTL3. Consistently, it was found that METTL3 staining was markedly reduced in cells expressing METTL3 3A and WTAP 2A (FIG. 11E).

To gain further insight into how ERK phosphorylation decreases METTL3 ubiquitination, experiments were conducted to determine if any ubiquitin ligases or deubiquitinases were identified in the CRISPR-based genomic screen. Notably, USP5 was identified as a potential positive regulator (FIG. 3D). It has been shown that mutant B-RAF activates certain deubiquitinases, including USP5. Co-immunoprecipitation showed that the association between METTL3 and USP5 was increased upon B-RAF transfection (FIG. 11F). To confirm this physical interaction, METTL3 and USP5 were co-transfected, followed by immunofluorescence analysis. As shown in FIG. 11G, METTL3 was found in the nucleus while B-RAF promoted USP5 to translocate into the nucleus, which explains why the interaction between METTL3 and USP5 was increased upon B-RAF transfection. Because USP5 is an enzyme that could prevent protein ubiquitination, experiments were conducted to further examine whether USP5 deubiquitinates METTL3. Overexpression of USP5 decreased ubiquitination (FIG. 3E) and stabilized METTL3 (FIG. 3F). Consistently, USP5 knockdown in A375 cells resulted in less METTL3 (FIG. 3G). Taken together, these data demonstrate that ERK activation translocates USP5 to cell nuclei, whereby it interacts with phosphorylated METTL3 to increase the stability of METTL3 by reducing its ubiquitination level.

### Example 4

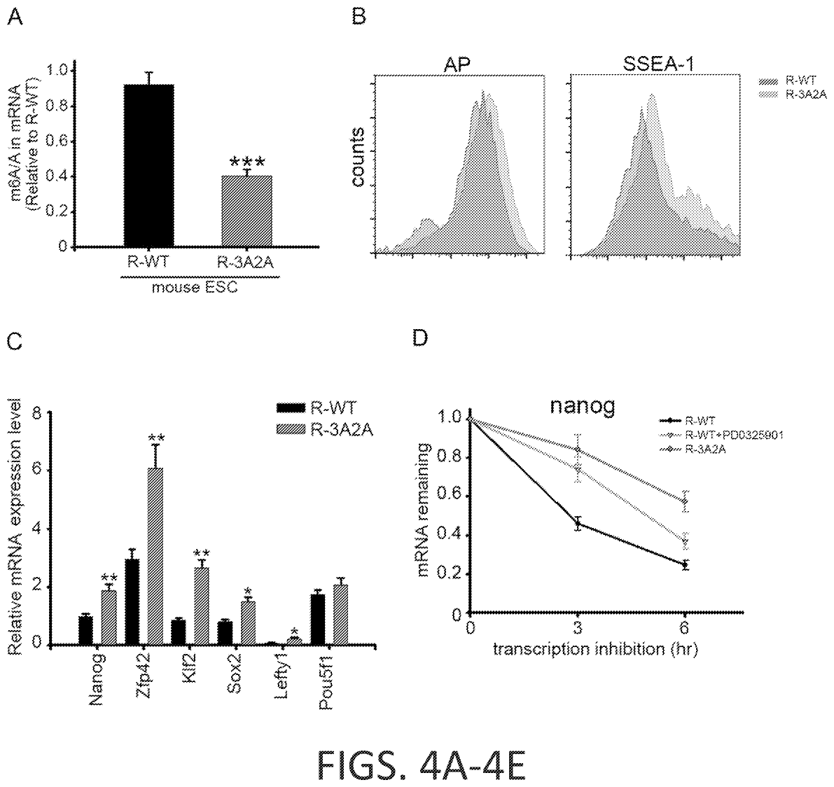

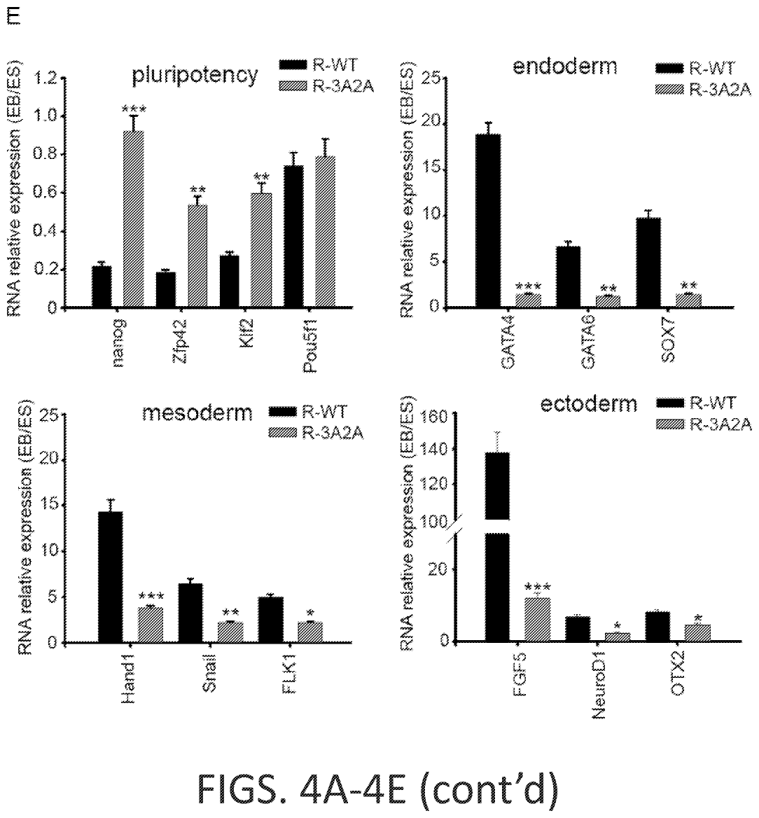

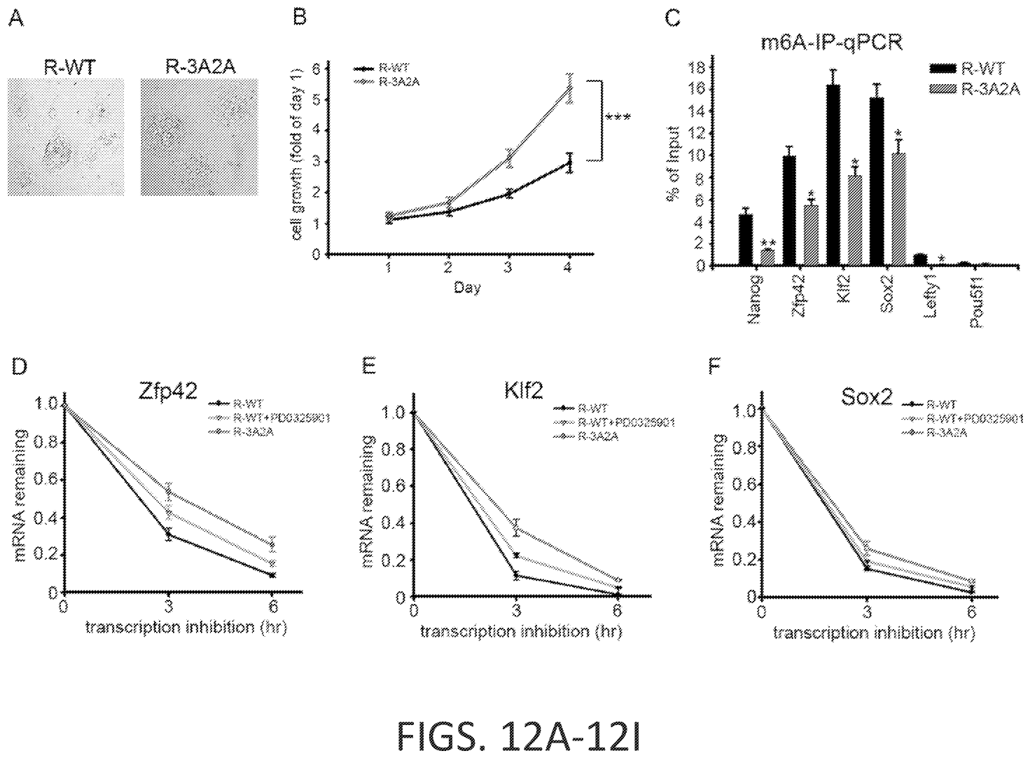

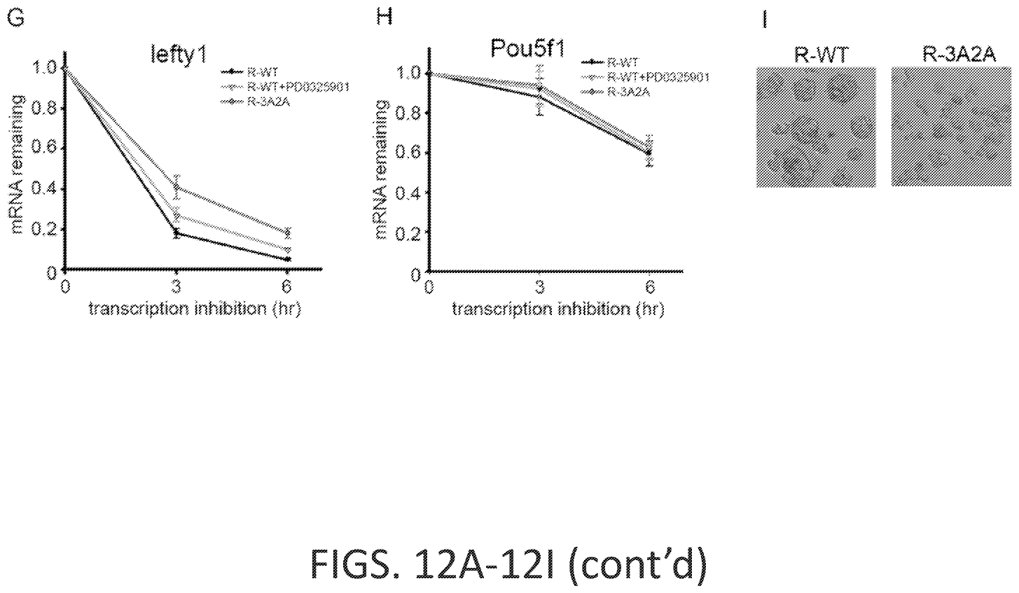

Phosphorylation of METTL3/WTAP by ERK Facilitates Resolution of Pluripotency. Because both ERK activation and METTL3 expression have been reported to be required for mESCs to exit the pluripotent state upon differentiation, experiments were conducted to investigate whether phosphorylation of METTL3/WTAP affects mESC fate. Wild-type METTL3 and WTAP (R-WT), or non-phosphorylatable METTL3 3A and WTAP 2A (R-3A2A) were re-expressed in homozygous Mettl3 knockout (KO) mESCs (FIG. 3A). Quantification of m⁶A by LC-MS/MS showed a significant reduction of mRNA m⁶A methylation in R-3A2A mouse ESCs (FIG. 4A). Experiments were then conducted to examine whether pluripotency of mESCs expressing R-3A2A was affected. Consistent with a previous report on Mettl3 KO in mESCs, R-3A2A mESC colonies were larger than R-WT mESCs and retained round, compact mESC colony morphology (FIG. 12A). Furthermore, R-3A2A mESCs exhibited higher alkaline phosphatase (AP) activity, stage specific embryonic antigen 1 (SSEA-1) expression (FIG. 4B) and increased proliferation (FIG. 12B). These observations support the notion that loss of METTL3 phosphorylation may facilitate trapping mESCs in the pluripotent state.

Mettl3-deficient mESCs fail to exit pluripotency despite differentiation cues, likely because loss of m⁶A impedes the degradation of pluripotency-promoting transcripts. Experiments were then conducted to examine reported m⁶A-methylated pluripotency factor transcripts, including Nanog, Zfp42, Klf2, Sox2, and Lefty1. Pou5f 1, which does not harbor m⁶A modification, was also used as a negative control. m⁶A-RIP-qPCR confirmed decreased m⁶A (FIG. 12C) and RT-qPCR indicated upregulation (FIG. 4C) of these m⁶A-labeled pluripotency transcripts in R-3A2A mESCs. Furthermore, after transcription arrest by actinomycin D treatment, these transcripts showed delayed turnover in both R-3A2A and R-WT mESCs treated with PD0325901 (FIG. 4D and S5D-H). These findings suggest that METTL3 phosphorylation controls the level of critical pluripotency regulators. Considering ERK activation is the primary stimulus for mESCs to exit self-renewal and acquire competence for differentiation, experiments were conducted to analyze the capacity for differentiation by transferring mESCs to differentiation media for embryoid bodies (EBs). R-3A2A mESCs generated smaller EB spheres (FIG. 12I), failed to repress pluripotent genes, and adequately up-regulated developmental markers (FIG. 4E). These results support the concept that the ERK-dependent phosphorylation of METTL3 and WTAP promotes mESC differentiation.

### Example 5

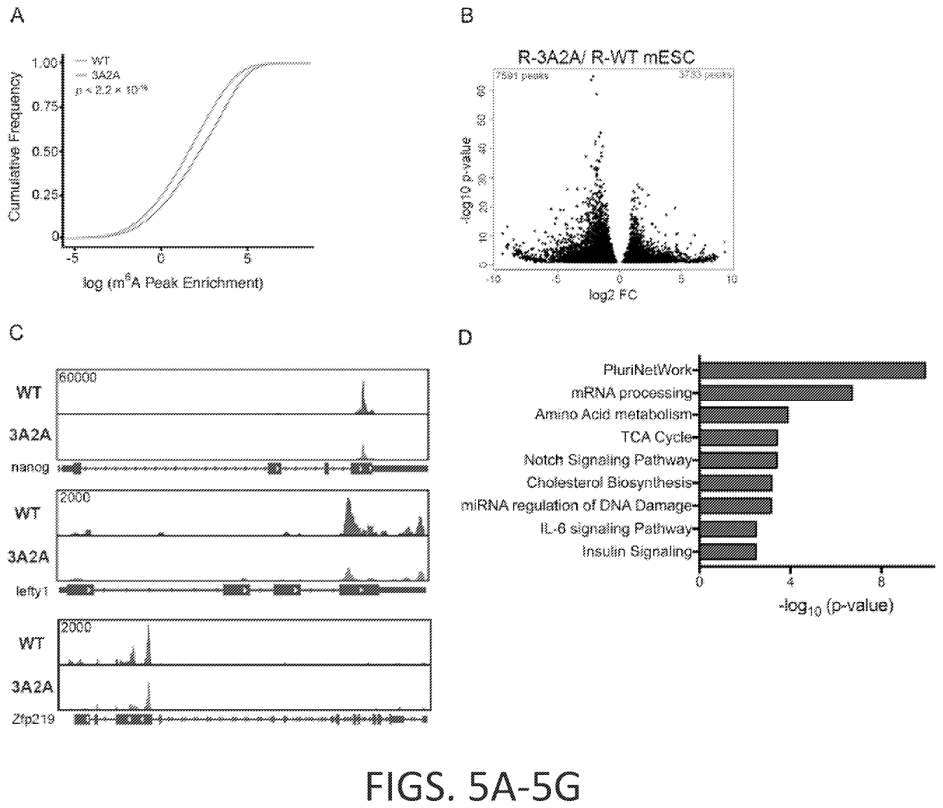

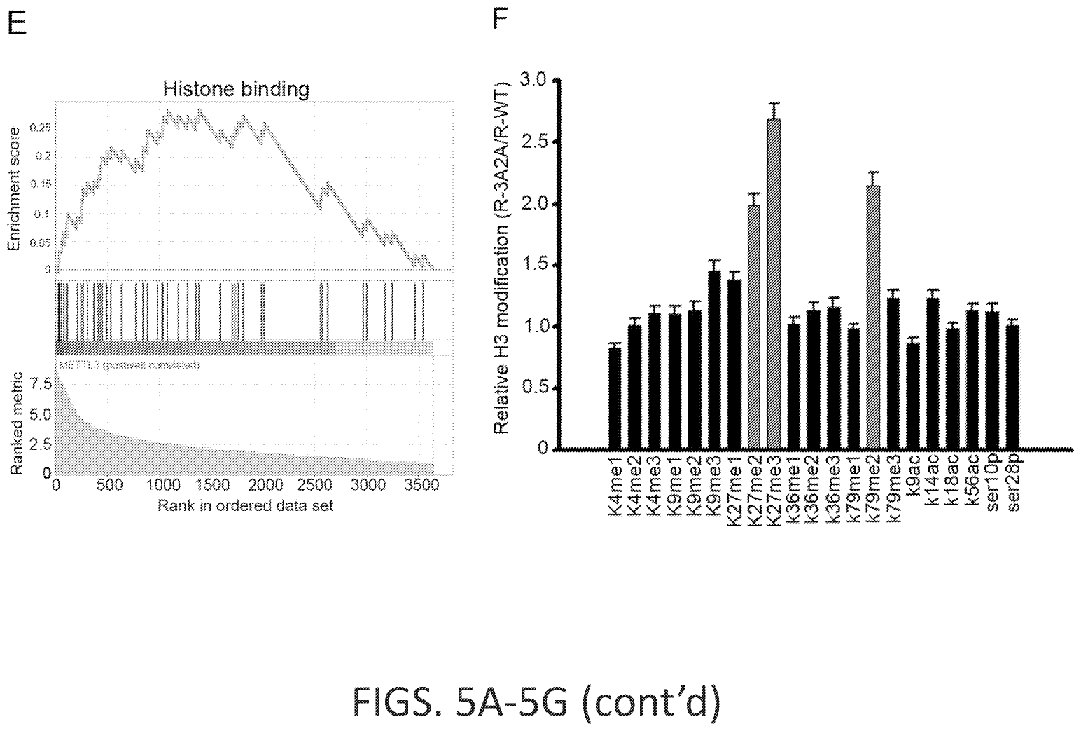

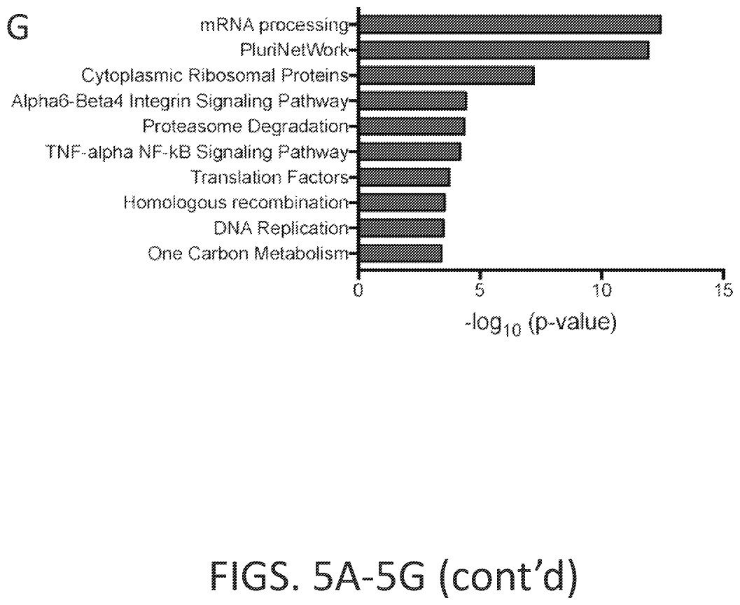

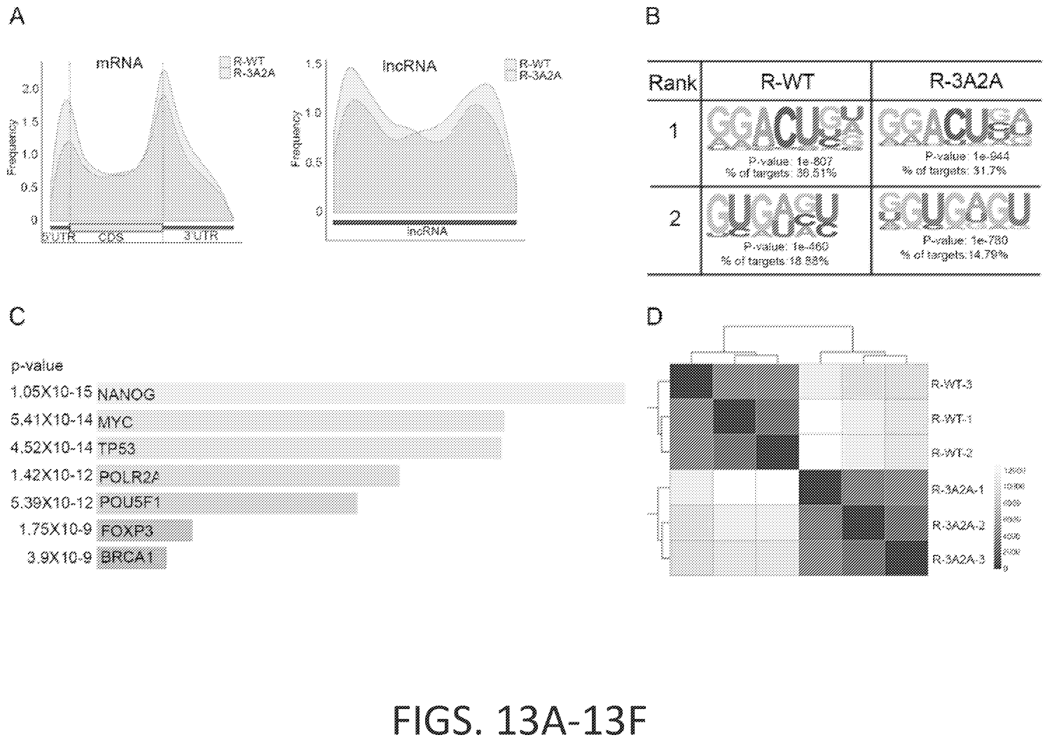

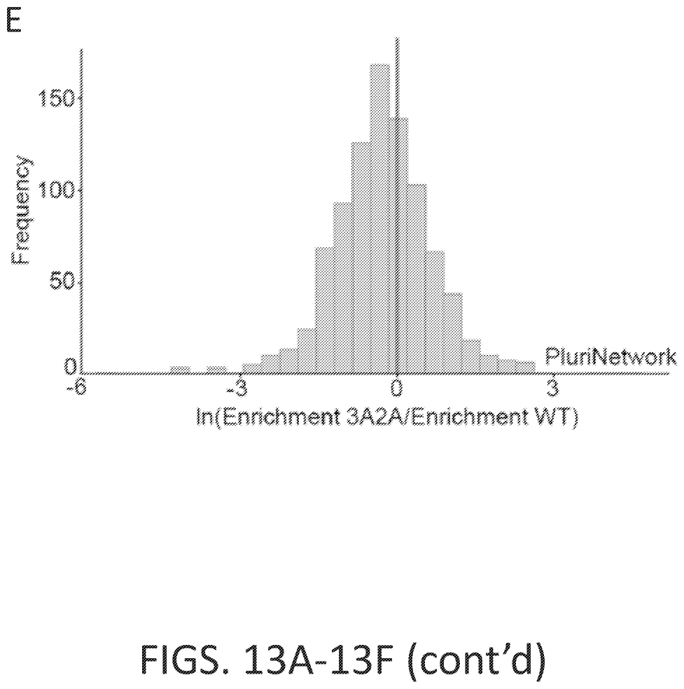

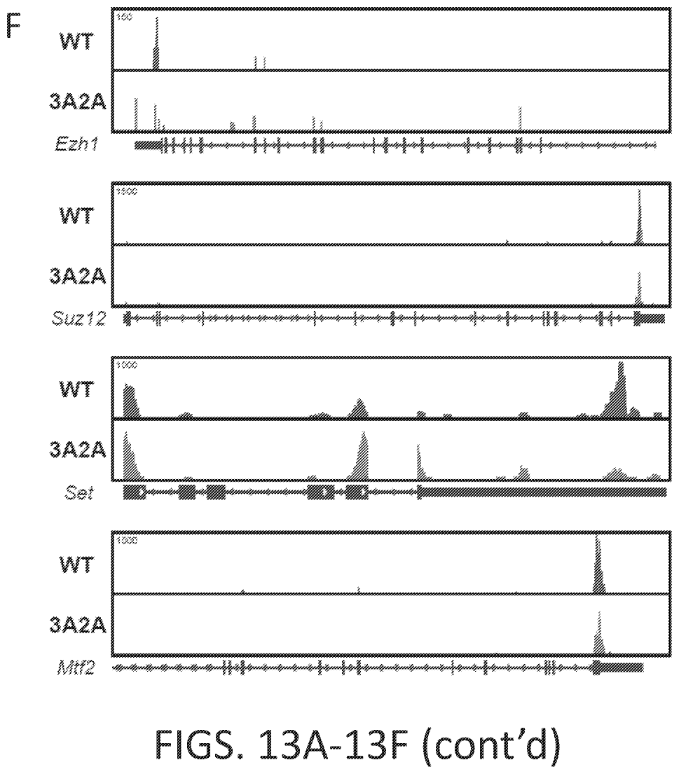

Transcripts Affected by Phosphorylation of Methyltransferase Complex in mESCs. To gain further insight into how the phosphorylation of the m⁶A methyltransferase complex affects the m⁶A-modified transcripts, the m⁶A methylome was mapped in mESCs. Comparison of the R-WT with R-3A2A mESCs revealed a global loss of methylation sites (FIG. 5A). Consistent with previous m⁶A-seq results, the m⁶A peaks identified are enriched near the start and stop codons and were characterized by the canonical GGACU motifs (FIGS. 13A-13B). Using a R-package “MeRIPtools,” which tests for m⁶A-IP enrichment using a binomial-distribution-based model, 7,591 m⁶A peaks were found that exhibited a decrease in the R-3A2A cells compared to the R-WT cells (FIG. 5B), such as modification sites in Nanog, Lefty1, and Zfp219 (FIG. 5C). (Data relating to genes identified by GSEA related to histone binding proteins, and data relating to significantly altered m6A peaks between R-WT and R-3A2A mESCs can be made available upon request.) The genes showing decreased m⁶A methylation significantly overlap with those functional gene sets important for pluripotency, including targets of Nanog and Myc (FIG. 13C). The transcripts exhibiting differential methylation were consistent between replicates (FIG. 13D) and enriched for gene ontology (GO) terms related to pluripotency, mRNA processing, and metabolism (FIG. 5D). Specially, many of the genes involved in the pluripotency showed reduced m⁶A methylation in R-3A2A when compared with R-WT mESCs (FIG. 13E).

To expand the observation of pathways or sets of genes that are enriched when comparing R-WT and R-3A2A mESCs, a functional class scoring approach (gene-set enrichment analysis, GSEA) was also performed besides GO analysis. GSEA showed enrichment of histone binding proteins (FIG. 5E; data relating to genes identified by GSEA related to histone binding proteins, and data relating to significantly altered m6A peaks between R-WT and R-3A2A mESCs can be made available upon request). Considering it has been reported that m⁶A regulates histone modifications in part by destabilizing mRNA of histone-modifying enzymes, an ELISA kit was used to compare 21 different Histone H3 modifications. As shown in FIG. 5F, H3K27me3 showed the most dramatic changes among all modifications. The PRC2 complex mediates methylation of lysine 27 on histone H3 to repress genes involved in the differentiation. Loss of m⁶A peaks was detected in several components of the PRC2 complex, including Ezh1, Suz12, Set, and Mtf2 (FIG. 13F). Furthermore, genes involving pluripotent network and mRNA processing were differentially expressed between R-WT and R-3A2A cells (FIG. 5G). These results demonstrate that phosphorylation of the m⁶A methyltransferase complex decreases H3K27me3 partially though regulating PRC2 complex, which contributes to activate differentiation-related genes.

### Example 6

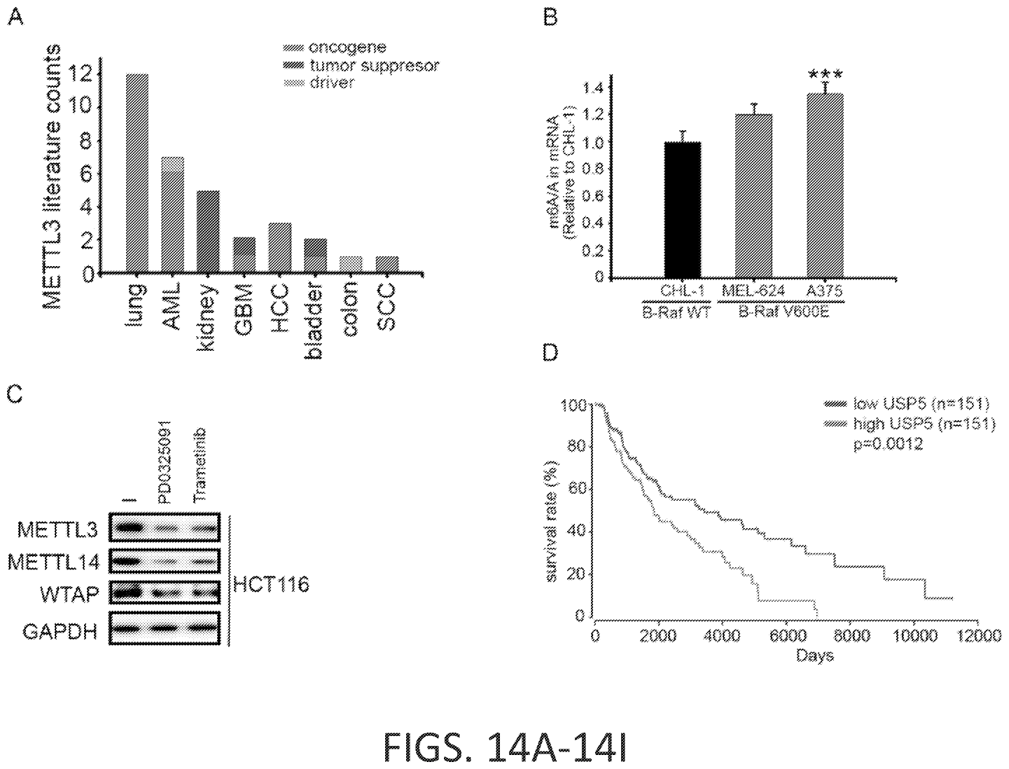

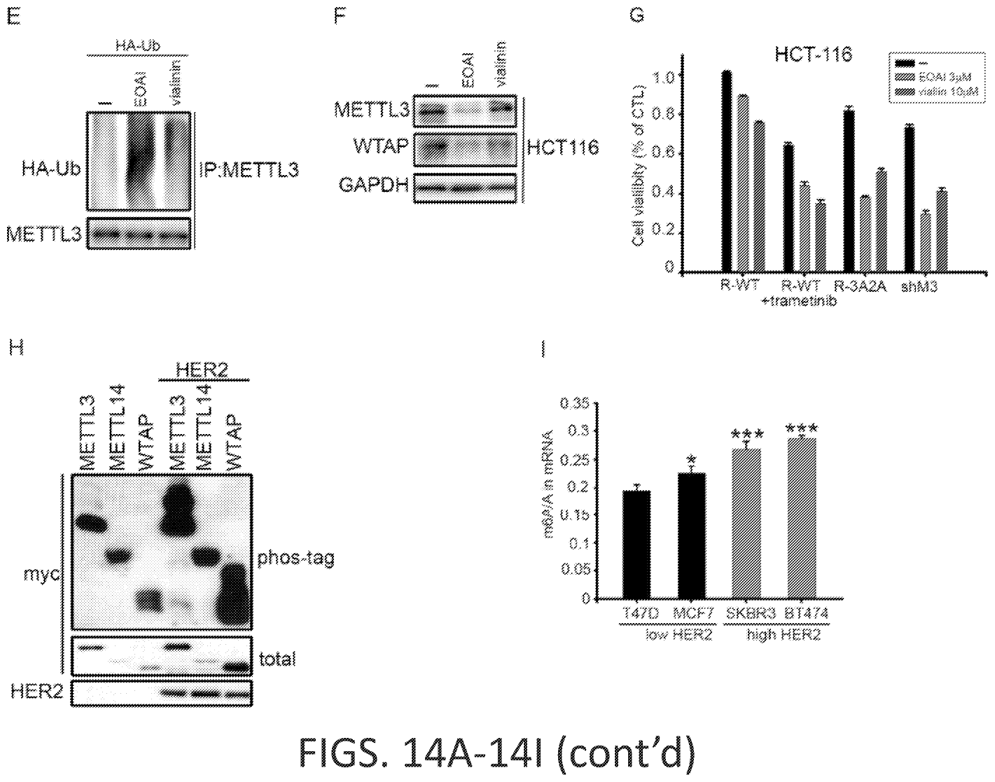

Phosphorylation of the m⁶A Methyltransferase Complex May Affect Tumorigenesis. As one of the most frequently mutated signaling pathways in cancer, the Ras/Raf/MEK/ERK signaling cascade has long been viewed as promising targets for cancer therapy. Given that phosphorylation of the m⁶A methyltransferase complex by ERK facilitates resolution of pluripotency in mESCs, experiments were conducted to further investigate whether the m⁶A methyltransferase complex can be similarly regulated in certain cancer cells. METTL3 knockdown is known to induce apoptosis and METTL3 overexpression could promote tumorigenesis in multiple cancer types. Using Cancermine (Lever et al., 2019), a literature-mined resource, it was determined that METTL3 could behave as an oncogene in many cancer types (FIG. 14A).

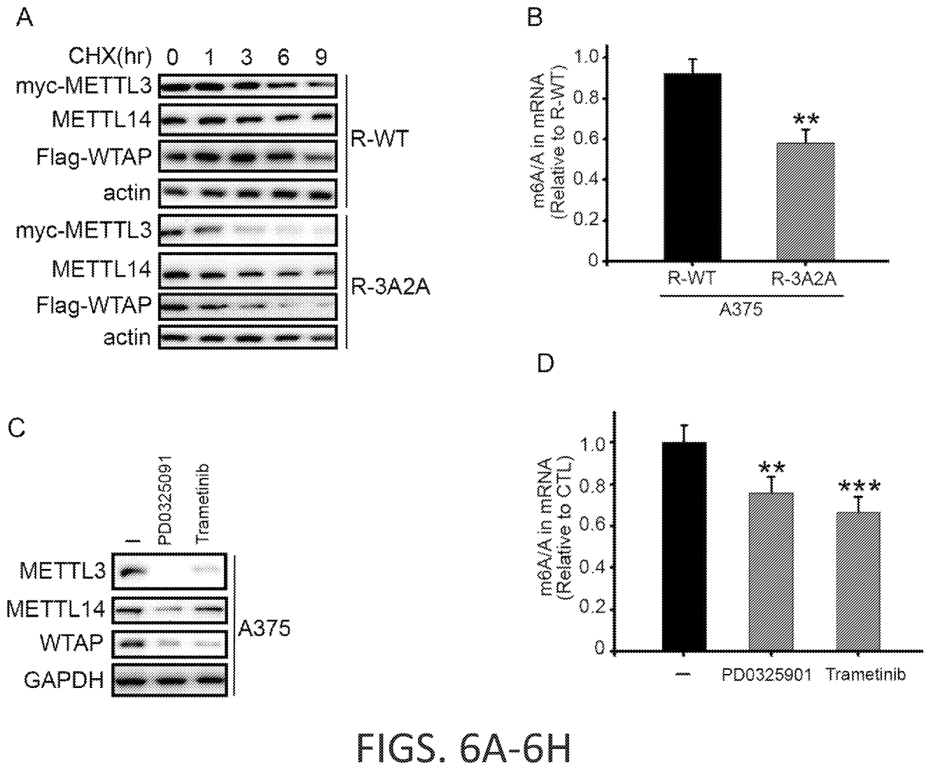

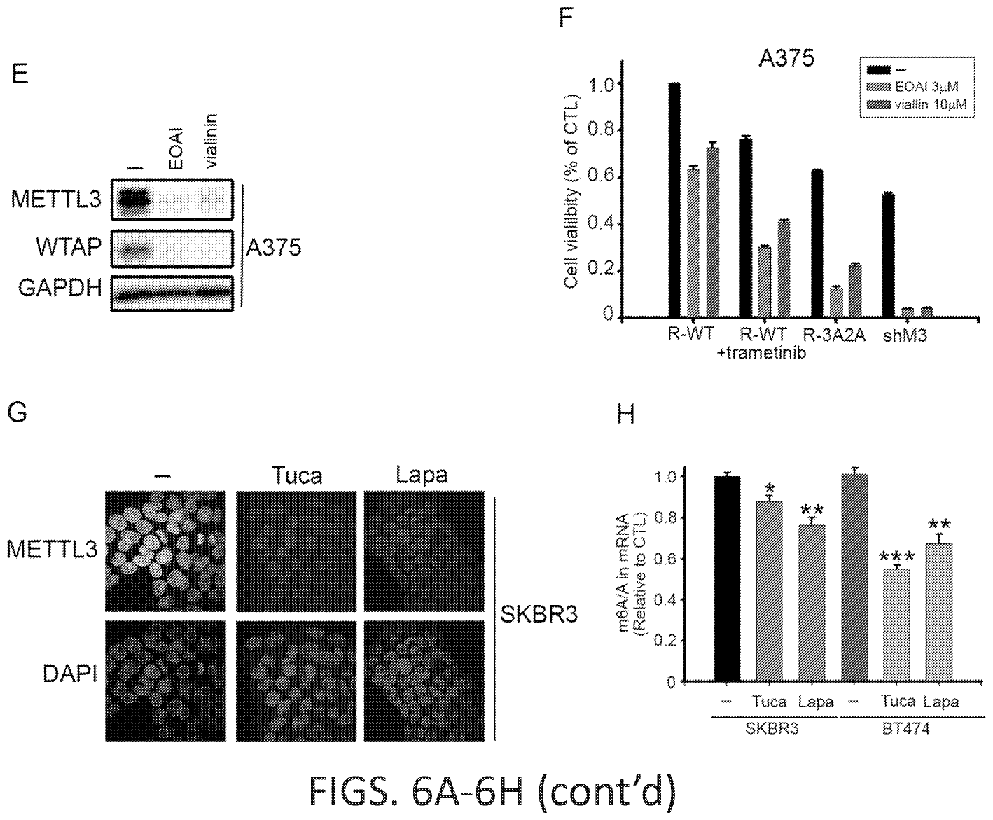

Experiments were conducted to first examine melanoma due to the high prevalence of constitutively active BRAF V600E mutation (50-60%) and clinical success with BRAF and MEK inhibitors. The m⁶A levels on polyA-tailed RNA are higher in the MEL-624 and A375 cells, which harbor a BRAF V600E mutation (FIG. 14B). As expected, the stability of the m⁶A methyltransferase complex was reduced for the R-3A2A A375 cells (FIG. 6A), which contributed to the overall lower m⁶A level on polyA-tailed RNA (FIG. 6B). MEK inhibitors PD0325901 and trametinib were found to reduce the protein levels of the m⁶A methyltransferase complex (FIG. 6C) and the overall mRNA m⁶A levels (FIG. 6D) in A375 melanoma cells. In addition, these two MEK inhibitors also decreased m⁶A methyltransferase complex level in HCT-116 cells, which is a colon cancer line that possesses the most common KRAS mutation (G12D) (FIG. 14C).

Because knockdown of USP5 increases METTL3 in A375 melanoma cells (FIG. 3F), the potential clinical relevance of USP5 was accessed. Melanoma patients with high USP5 had shorter overall survival (FIG. 14D). Two structurally unrelated USP5 inhibitors, EOAI3402143 and vialinin, were employed to evaluate the effect of USP5 on the METTL3 level in melanoma cells. It was observed that these two USP5 inhibitors increased ubiquitination of METTL3, resulting in decreased METTL3 protein level (FIG. 6C and FIGS. 14E-14F). Furthermore, MEK inhibition, R-3A2A METTL3, or METTL3 knockdown can sensitize melanoma and colon cancer cells to USP5 inhibition (FIG. 6F and FIG. 14G), supporting a connection between USP5 and METTL3, and suggesting USP5 inhibition could be a strategy to deplete METTL3 in cancers.

Lastly, considering that HER2 expression phosphorylates METTL3 and WTAP (FIG. 8C and FIG. 14G) and m⁶A levels are higher in the HER2-overexpressed SKBR3 and BT474 cells (FIG. 14H), experiments were conducted to investigate whether inhibition of HER2 could affect m⁶A methylation. Two HER2 inhibitors, tucatinib and lapatinib, could reduce METTL3 protein level and cellular mRNA m⁶A methylation in HER2-positive breast cancer (FIGS. 6G-6H). Overall, these data support that ERK-dependent METTL3 stabilization affects cellular mRNA m⁶A methylation which could contribute to tumorigenesis.

### Example 7

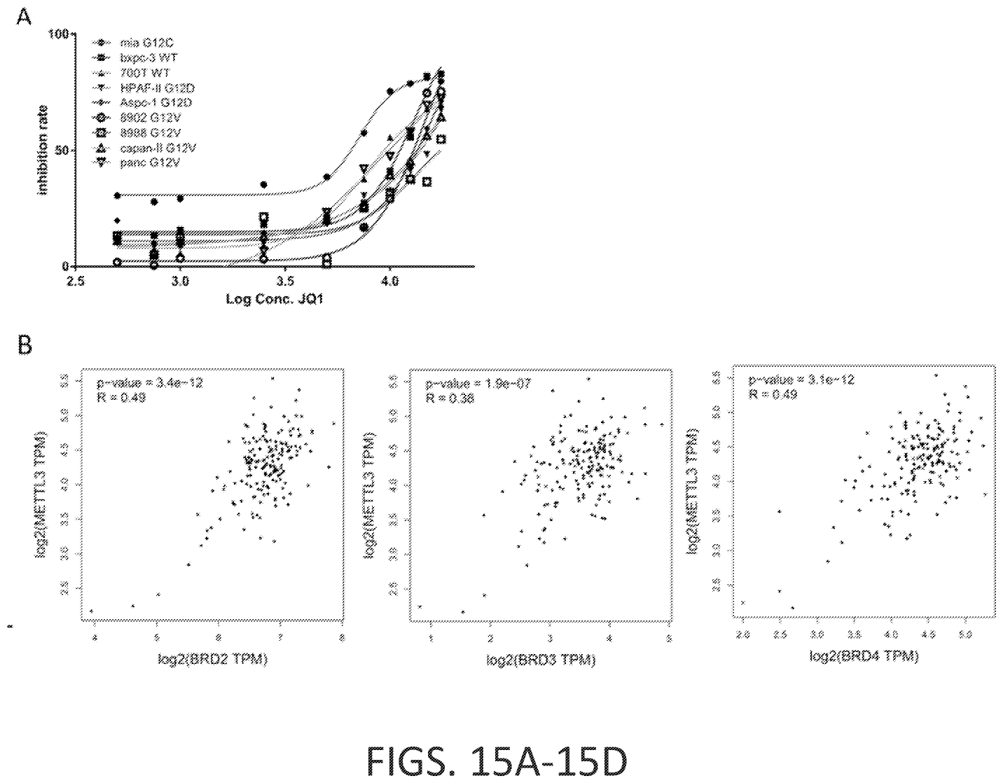

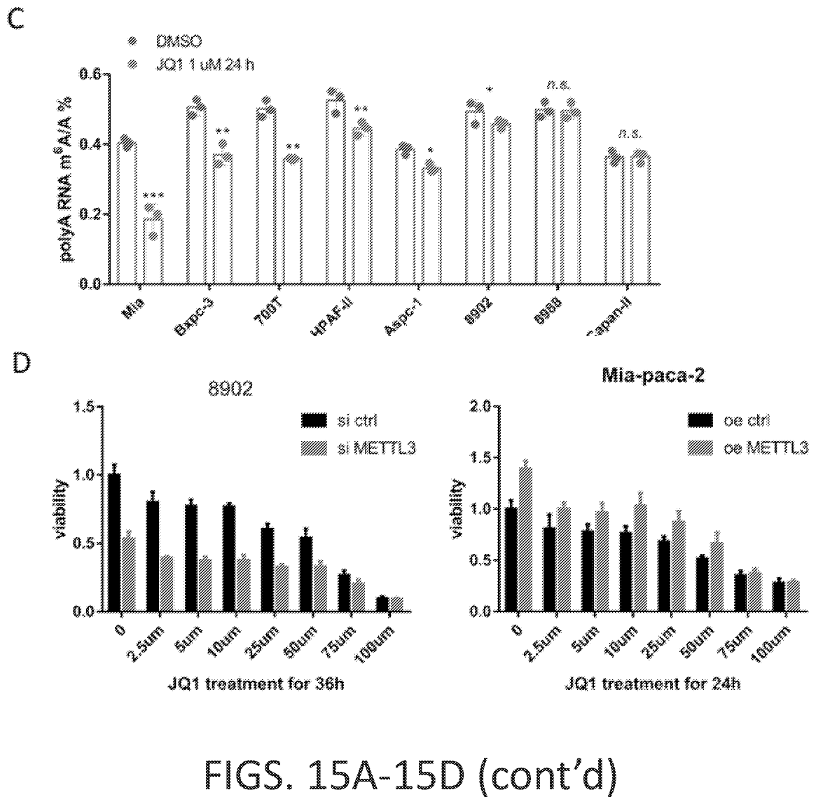

Bromodomain and extraterminal domain (BET) family of proteins. The bromodomain and extraterminal domain (BET) family of proteins have been investigated as a potentially effective therapeutic target for treating PDAC tumors. JQ1 inhibits BET protein function by binding to the domain of BET that interacts directly with acetylated lysine residues on specific histones, thereby condensing the chromatin globally and decreasing expression of proteins that rely on BET-dependent mechanisms for transcription. A panel of 9 PDAC cell lines was used to test the anticancer activity of JQ1, and it was found that the inhibition effect of JQ1 varies among different cell types (FIG. 15A). There is a report that histone modification H3K36Me3 guides m⁶A RNA modification co-transcriptionally and preliminary data indicates that chromatin openness correlates to m⁶A RNA modification. As JQ1 could reduce the global chromatin openness through the inhibition of BET family proteins and the expression of multiple BET family proteins show high correlations with METTL3 in PDAC based TCGA database (FIG. 15B), experiments were conducted to test the polyA RNA m⁶A levels in the PDAC cell lines that subjected to the inhibition rate test. The mass back results showed that JQ1 treatment leads to dramatically decreased polyA RNA m⁶A in JQ1-sensitive cells while a moderate to negligible polyA RNA m⁶A changes in JQ1-insensitive cells (FIG. 15C). Knockdown of METTL3 could synergistically reduce the viability of JQ1-insensitive cells together with JQ1 treatment, while overexpression of METTL3 in JQ1-sensitive cell inhibits the effect of JQ1 treatment (FIG. 15D).

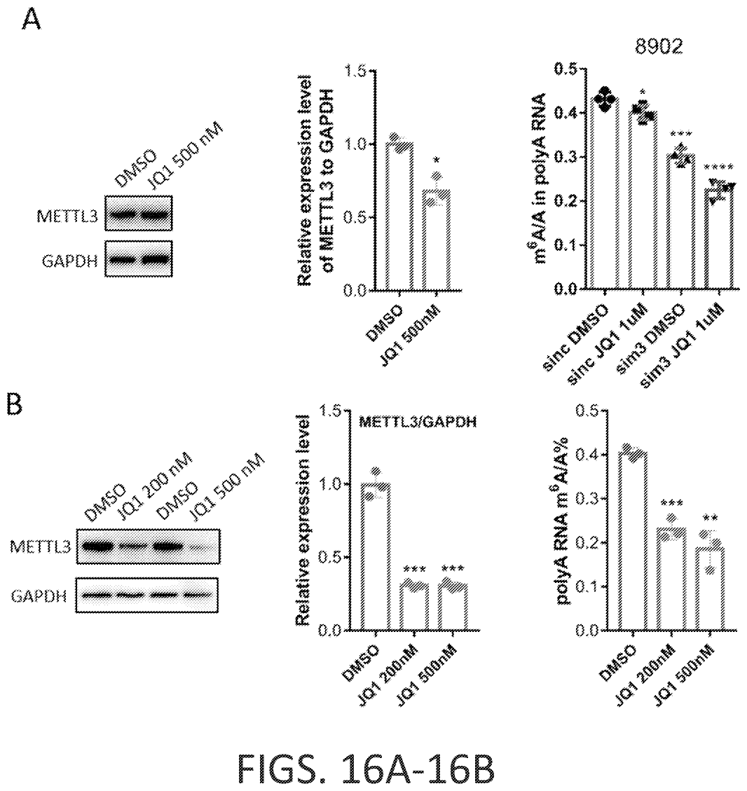

The 8902 cell line was used as a JQ1-insensitive cell and the Mia cell as a JQ1-sensitive cell to further investigate the potential synergistic effects of JQ1 and knockdown of METTL3. The results showed that in the JQ1-insensitive cell, JQ1 treatment has a moderate effect on METTL3 RNA level and less of an effect on METTL3 protein level. However, JQ1 treatment after METTL3 knockdown further reduced the polyA RNA m⁶A, which indicates JQ1 treatment may regulate the accessibility of METTL3 towards its substrate (FIG. 16A). Additionally, in the JQ1-sensitive cells, JQ1 treatment leads to dramatic decreases of both METTL3 RNA level and protein level and thus polyA RNA m⁶A level in a dose-dependent manner (FIG. 16B).

### Example 8

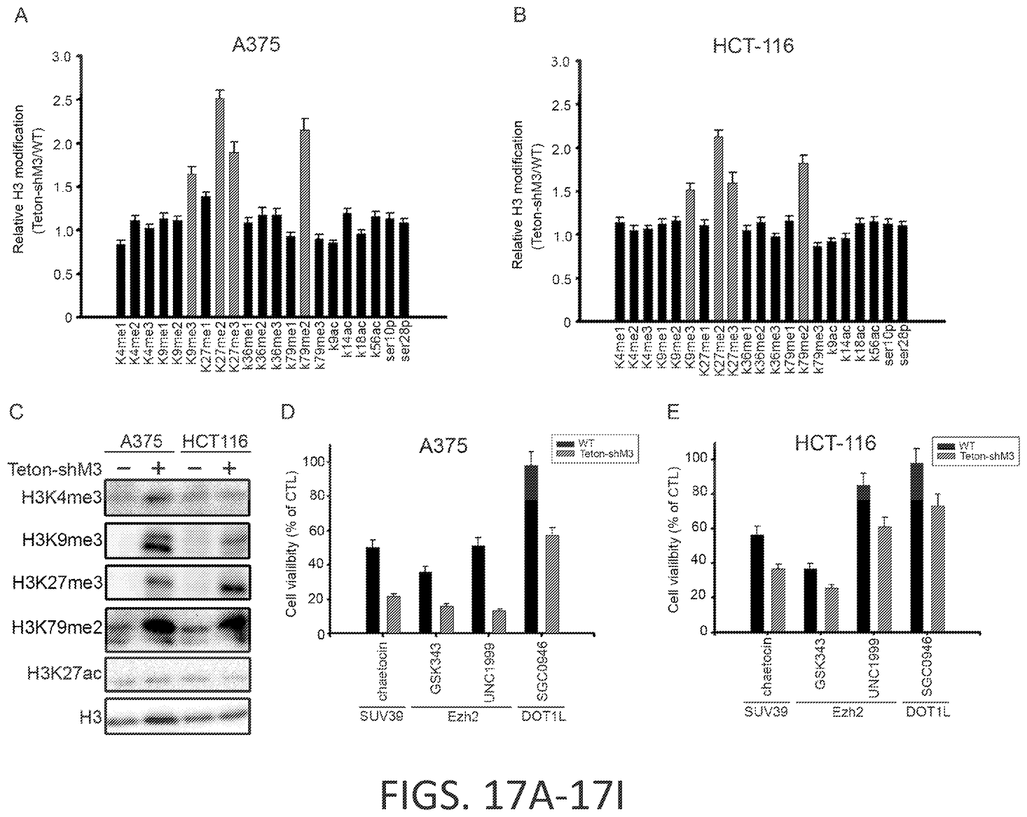

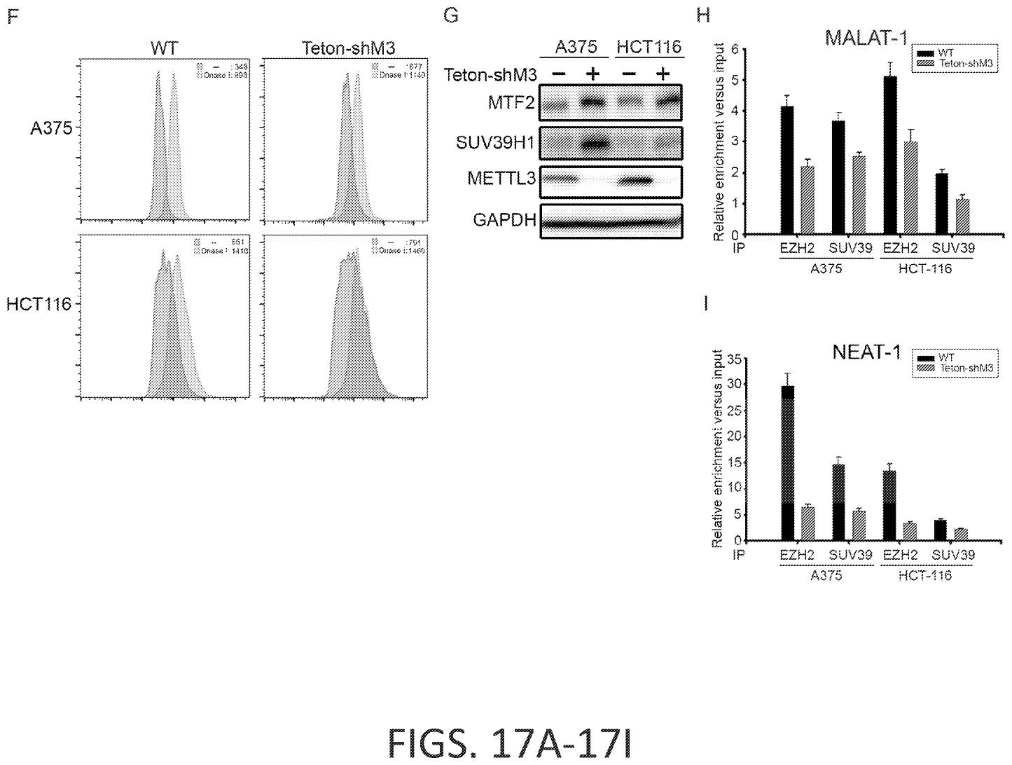

Histone methyl transferase (HMT) activity. N6-methyladenosine (m⁶A), catalyzed by the methyltransferase complex consisting of Mettl3 and Mettl14, is the most abundant RNA modification in mRNAs and participates in diverse biological processes. The mechanisms by which m⁶A modification affects gene expression are being investigated. In a previous study, gene-set enrichment analysis (GSEA) showed that histone-binding proteins were enriched when comparing m⁶A-labled genes in wild type and mutant METTL3 mESC. Therefore, an ELISA kit was used to compare 21 different Histone H3 modifications in melanoma (A375) and colon cancer cells (HCT116). As shown in FIGS. 17A-17B, METTL3 KD increased H3K9me3, H3K27me2, H3K27me3, and H79me2, among all modifications. Western blots further confirm H3K9me3, H3K27me3, H79me2 are significantly increased compared to H3K4me3 and H3K27ac (FIG. 17C). Those increased histone methylations result in susceptibility to relevant histone methyl transferase (HMT) inhibitors (FIGS. 17D-17E); chaetocin (SUV39H1-dependent H3K9me3), GSK343 and UNC199 (EZh2-dependent H3K27me2 and H3K27me3), and SGC0946 (DOT1L-dependent H79me2). These results demonstrate that HMT could be a therapeutic target in METTL3-low cancer and indicate a potential synergism between METTL3 and HMT inhibitors.

H3K9me3 and H3K27me3 are repressive chromatin markers that correlate with transcriptional repression. Therefore, a DNaseI-TUENL assay was used to measure chromatin accessibility. As show in FIG. 17F, the effect of DNaseI was decreased when METTL3 was knockdown, suggesting METTL3 loss leads to closed chromatin. Experiments were then conducted to investigate how m⁶A regulates histone modification. Expression levels of H3K9 and H3K27 methyltransferase complex were first evaluated. Consistent with a previous observation in mESC, MTF2 (DNA-binding H3K27 recruiter) and SUV39H1 (H3K9me3 methyltransferase) were increased in METTL3 KD melanoma and colon cancer cells. Ezh2 recently emerged as a critical RNA-binding subunit with a general preference of 1nRNA. The interaction between MALAT1 (a well-known m⁶A-labeled 1nRNA) and PRC2 complex has been reported to release the target genes from repressed status (in polycomb bodies) to activated form (in interchromatin granules) in response to stimulation of growth signals. It was observed that knockdown of METTL3, which decreased m⁶A in MALAT-1 and NEAT-1, leads to the release of EZH2 and SUV39H1, which may contribute to the increase of H3K27me3 an H3K9me3 (FIGS. 17H-17I).

### Example 9

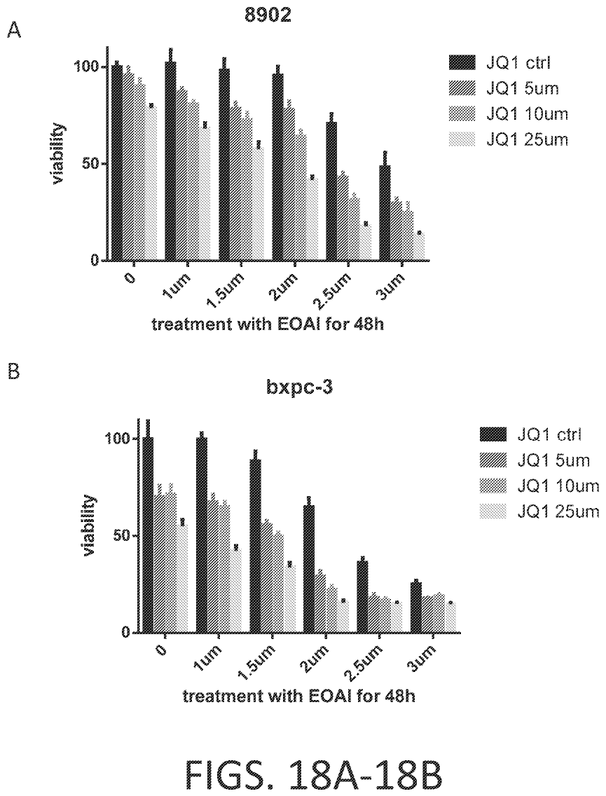

USP5 Inhibition. EOAI is a USP5 inhibitor that could lead to a decreased level of METTL3 protein. When applied together in JQ1-insensitive cells (e.g., 8902 cells), the combination of 5 μmol JQ1 and 1.5 μmol EOAI exhibited comparable effect on cell viability as a single dose of 10 μmol JQ1 or 2.5 μmol EOAI (FIGS. 18A-18B). This demonstrates that reduced METTL3 has synergistic effects with JQ1 treatment. Additionally, when the combination of EOAI and JQ1 was applied to JQ1 sensitive cells, synergistic effects were also observed (FIGS. 18A-18B).

### Example 10

USP5 inhibition Leads to Increased Ubiquitination of METTL3. Through performing a CRISPR-based genomic screen using GGACU motif-circular RNA-GFP reporter, USP5 was identified as a potential positive regulator of m⁶A pathway. It has been shown that mutant B-RAF activates certain deubiquitinases, including USP5. Co-immunoprecipitation showed that the association between METTL3 and USP5 was increased upon B-RAF transfection (FIG. 3D). Because USP5 is an enzyme that could prevent protein ubiquitination, experiments were further conducted to examine whether USP5 deubiquitinates METTL3. Overexpression of USP5 decreased ubiquitination and stabilized METTL3. Consistently, USP5 knockdown in A375 cells resulted in less METTL3 (FIGS. 3E-3F). Because knockdown of USP5 decrease METTL3, the potential clinical relevance of USP5 was further accessed using two structurally unrelated USP5 inhibitors, EOAI3402143 (EOAI) and vialinin. It was found that these two USP5 inhibitors increased ubiquitination of METTL3, resulting in decreased METTL3 protein level (FIGS. 14E-14F).

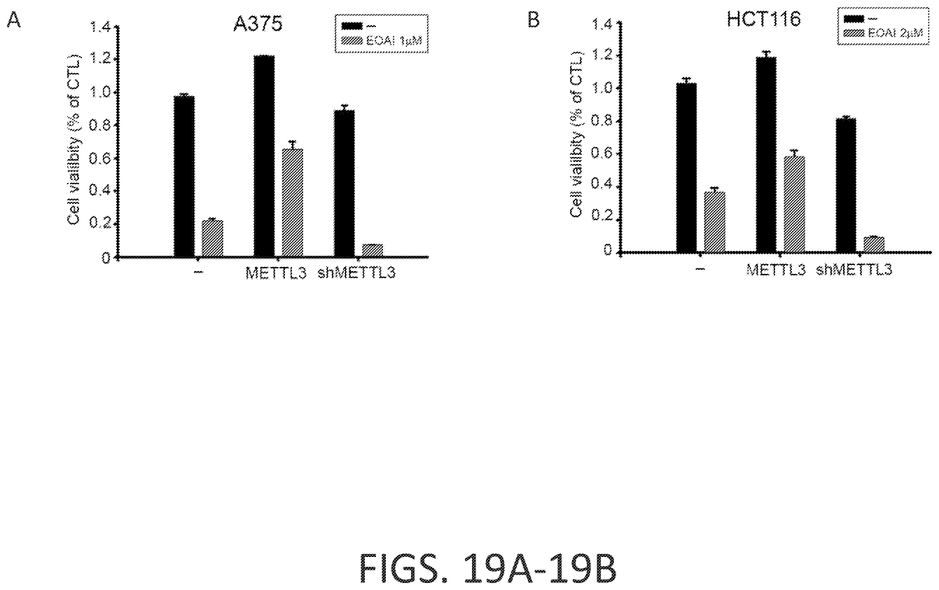

Furthermore, overexpression of METTL3 attenuated, while knockdown of METTL3 sensitized, melanoma and colon cancer cells to USP5 inhibitors EOAI, supporting the connection between USP5 and METTL3 (FIGS. 19A-19B). In a previous study, gene-set enrichment analysis (GSEA) showed that histone-binding proteins were enriched when comparing m⁶A-labled genes in wild type and mutant METTL3 mESC. Therefore, an ELISA kit was used to compare 21 different Histone H3 modifications in melanoma (A375) and colon cancer cells (HCT116). METTL3 KD increased H3K9me3, H3K27me2, H3K27me3, and H79me2 among all modifications (FIGS. 17A-17B). Those increased histone methylations when METTL3 was knocked down resulted in susceptibility to relevant hi stone methyl transferase (HMT) inhibitors; chaetocin (SUV39H1-dependent H3K9me3), GSK343 and UNC199 (EZh2-dependent H3K27me2 and H3K27me3), and SGC0946 (DOT1L-dependent H79me2) (FIGS. 17D-17E),

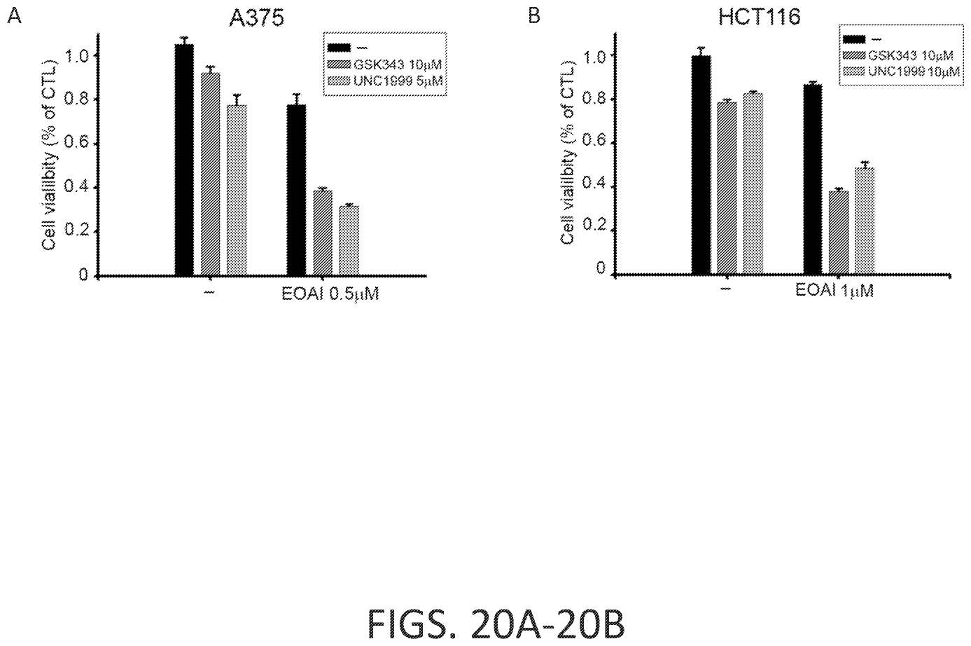

Since synergism between METTL3 KD and Ezh2 inhibitors was observed, further experiments were conducted to examine the effect of the combination of the USP5 inhibitor, EOAI, with Ezh2 inhibitors, GSK343 and UCN1999. The results shown in FIGS. 20A-20B demonstrate synergism between USP5 and Ezh2 inhibitors in ERK-activating melanoma and colon cancer.

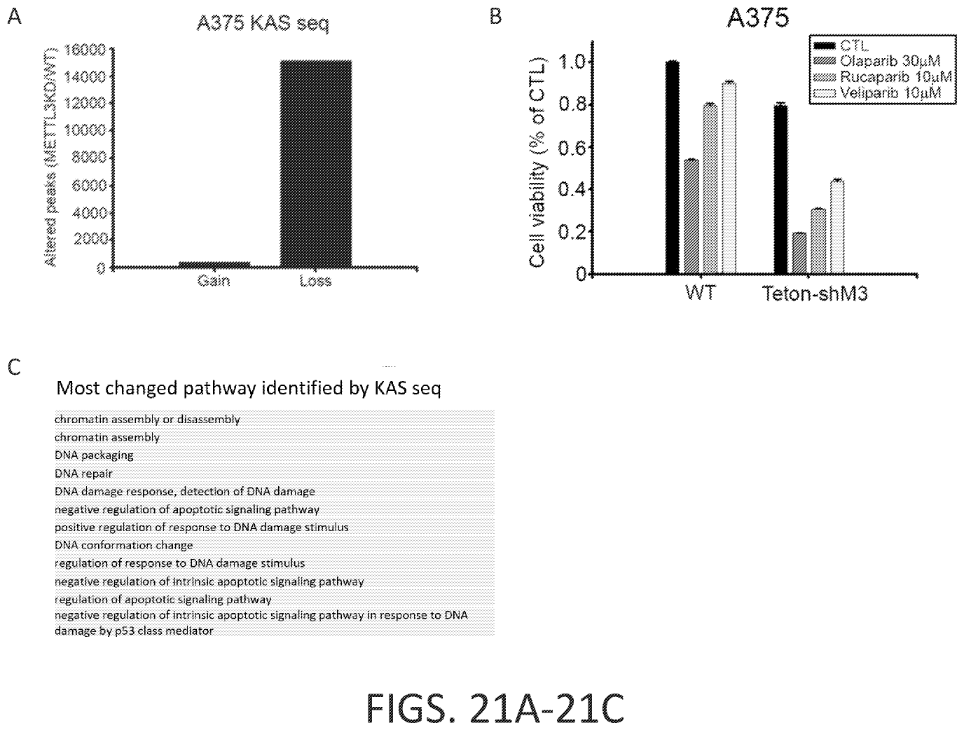

Similar experiment were conducted to investigate the involvement of poly(ADP-ribose) polymerase 1 (PARP1) in chromatin stability. Considering METTL3 KD affects chromatin status, with possible effects on transcription dynamics, the kethoxal-assisted single-stranded DNA sequencing (KAS seq) was further used to investigate global transcription dynamics. Interestingly, the analysis identified peak loss when METTL3 was knocked down. GO analysis showed the enrichment of DNA damage pathway from the KAS seq. It has been reported that PARP is required to m⁶A accumulation during DNA damage. Similar experiment were therefore conducted to investigate the involvement of poly(ADP-ribose) polymerase 1 (PARP1) in chromatin stability. The results in FIGS. 21A-21C demonstrate synergistic effects on cell viability using a combination of METTL3 KD and PARP inhibitors (olaparib, rucaparib, and veliparib).

### Example 11

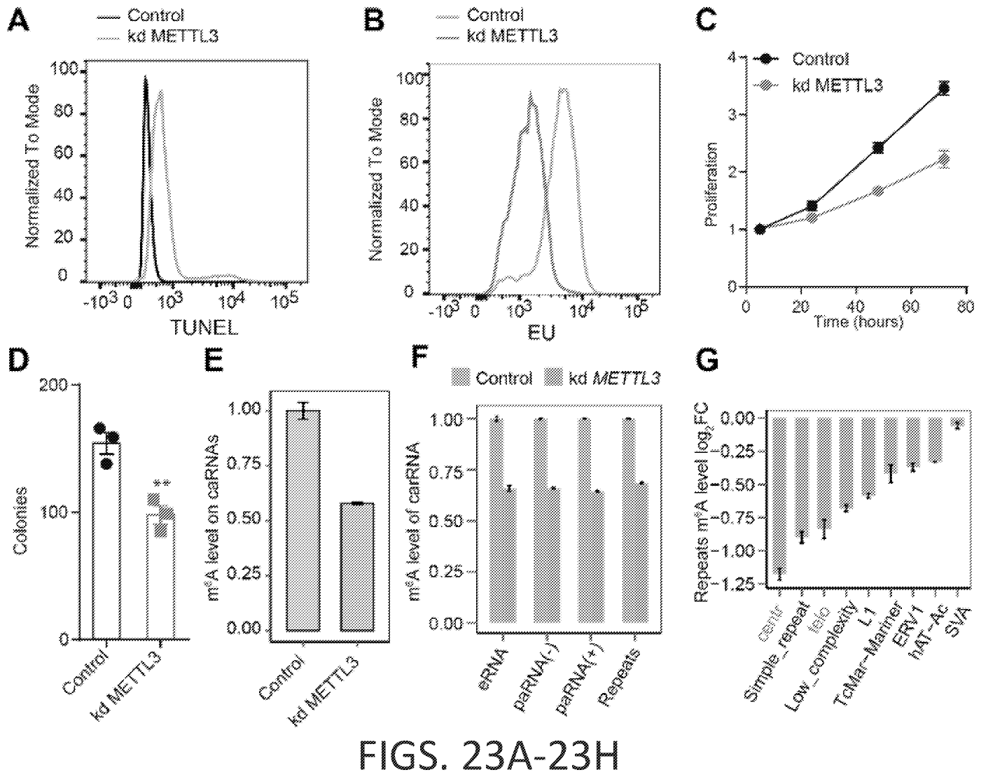

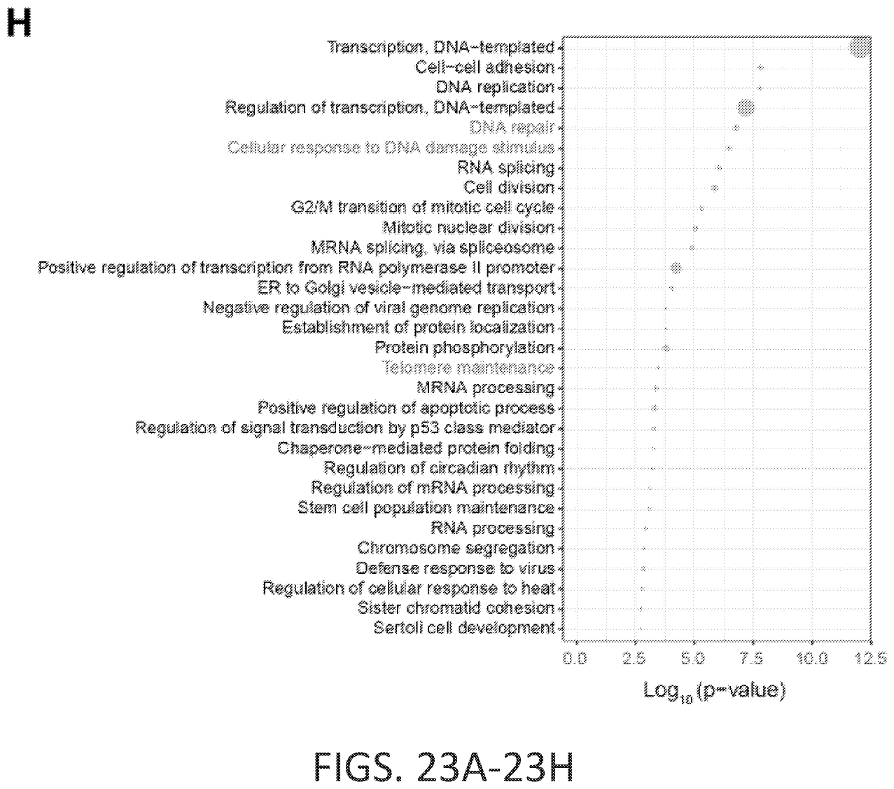

Genomic instability is a characteristic of many human cancers, and could involve defected DNA damage repair. If METTL3-mediated caRNA methylation plays important roles in DNA damage repair, then inhibition of METTL3 could preferentially affect tumors associated with genomic instability. Data provided herein demonstrated that METTL3 knockdown in A375 melanoma cancer cells triggered dsDNA breaks and caused apoptosis (FIG. 23A), accompanied with decreased transcription (FIG. 23B) and suppressed cell proliferation (FIG. 23C) and growth (FIG. 23D). Next, caRNA m⁶A-seq was performed, and a global decreased m⁶A level was observed on both caRNA (FIG. 23E) and carRNA (FIG. 23F). In addition, when repeat families were ranked according to their m⁶A level changes upon METTL3 knockdown, centromere RNA and telomere RNA were identified as the most responsive (FIG. 23G). Studies have shown that centromeric RNAs are closely associated with centromeric chromatin which is dependent on nucleoprotein complex assembly and critical for cell cycles. Misregulation of centromeric RNA can cause defective centromeric protein recruitment (such as CENP-A), thus affecting the chromatin stability. Moreover, when genes with upstream carRNA methylation-fold-changes of more than 1.5 were studied, they are mainly involved in regulation of DNA repair, cellular response to DNA damage stimulus and telomere maintenance (FIG. 23H). Together, these observations demonstrated that METL3 knockdown affected these chromatin-related carRNAs, which contributed to the increased DNA damage previously observed.

### Example 12

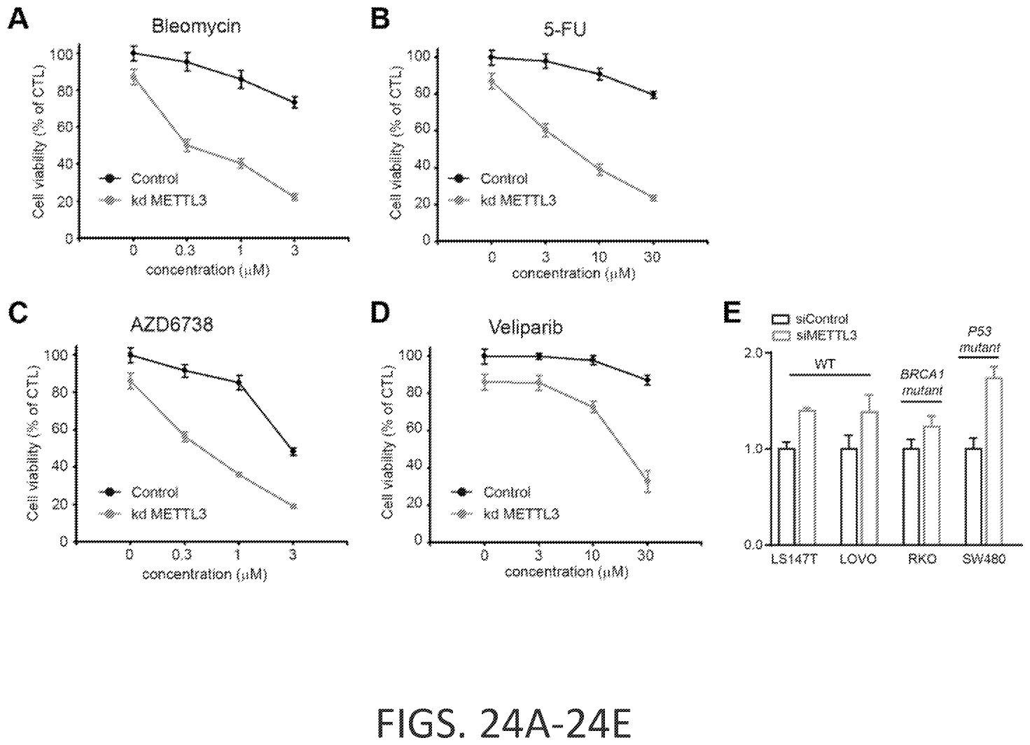

Next, the potential for synergistic effects with other agents was evaluated. A375 cancer cells were treated with DNA damage agents or inhibitors of specific DNA repair pathways (e.g., DNA damage repair modulators). METTL3 knockdown in A375 cells showed synergistic effects when treated with two DNA damage agents: Bleomycin, an ionizing radiation drug which induces dsDNA breaks (FIG. 24A), and 5-FU, an antimetabolite which interferes with nucleotide metabolism and DNA synthesis (FIG. 24B); the results support that METTL3 knockdown affects DNA damage repair pathways. Cancer cells were then treated with specific DNA damage repair inhibitors. Results demonstrated that METTL3 knockdown cells showed more severe cell death that controls without METTL3 perturbation after incubation with two inhibitors: AZD6738, an ATR inhibitor, central to the cell cycle signaling checkpoint (FIG. 24C), and Veliparib, a PARP inhibitor, responsible for DNA break detection and DNA repair machinery recruitment (FIG. 24D). These data collectively demonstrate that METTL3 plays significant roles in the dsDNA damage repair signaling such as homologous recombination (HR) and non-homologous end joining (NHEJ) pathways. Moreover, the effects of METTL3 knockdown on DNA damage in different colon cancer cells lines including p53 or BRCA1 mutant were investigated, and it was observed that the p53 mutant cancer cells shown more severe DNA damage upon knockdown METTL3 (FIG. 24E), further suggesting a combination of DNA damage drugs with depletion of METTL3 as a potential new therapeutic strategy in different human cancers.

### 4. Materials and Methods

mESC culture and differentiation. mESCs were generated, maintained, and differentiated essentially as previously described (Geula et al., 2015). METTL3 knockout mESCs were kindly provided by Dr. Howard Y. Chang (Stanford University) and regularly tested negatively for mycoplasma contamination. Established ESC clones were genotyped by PCR and validated as Mettl3-deficient by qPCR and Western blot. mESCs were cultured on mitomycin C-treated mouse embryonic fibroblasts in ES medium containing DMEM supplemented with 15% FBS, 1 mM L-glutamine, 0.1 mM mercaptoethanol, 1% non-essential amino acid , 1% Pen/Strep, nucleosides 1,000 U/ml leukemia inhibitory factor, 3 μM CHIR99021 and 1 μM PD0325901. For embryoid body (EB) differentiation, 5×10⁶ ESC were disaggregated with trypsin and transferred to non-adherent suspension culture dishes and cultured in MEF medium (DMEM supplemented with 1% L-Glutamine, 1% Non-essential amino acids, 1% penicillin/streptomycin and 15% FBS) for 8 days.

Cell Culture. HeLa, 293T, 293TN, A375, CHL-1, MEL-624 cells were maintained in DMEM supplemented with 10% FBS and 1% Pen/Strep. MCF-7, T47D, SKBR3, and HCT-116 cells were maintained in RPMI supplemented with 10% FBS and 1% Pen/Strep. BT474 cells were maintained in RPMI supplemented with 20% FBS and 1% Pen/Strep.

Plasmids. The circRNA reporters containing split GFP with a m⁶A motif were kindly provided by Z. Wang (Chinese Academy of Science, Shanghai, China) and subcloned into pCDH- CMV-MCS-EFlα-RFP (System bioscience, CD512B-1). The CRISPR knockout pooled library (#1000000048), METTL3 (#53739), METTL14 (#53740), WTAP (#53741), pKMyc (#19400), Flag-ATM (#31985), ATR (#31611), Flag-IKKe (#26201), HA-GSK-3β (#14754), ERK1 (#23509), ERK2 (#23498), B-Raf V600E (#17544), pMD2.G (#12259) and psPAX2 (#12260) were ordered from Addgene. Flag-IKKα, Flag-IKKβ, HA-AKT, Flag-mTOR, HA-MEKDD, HA-CDC2, FAK, EGFR, HER2 V659E, HA-ubiquitin, pCMV5-HA, and pCMV5-Flag were kindly provided by M. C. Hung (China Medical University, Taichung, Taiwan). pLightSwtich R01_3′UTR and Nanog 3′UTR were ordered from Switchgear Genomics. Mouse METTL3 (MR209093), mouse WTAP (MR216877), and USP5 (RC224191) were purchased from Origene. METTL3 (human and mice), METTL14, and WTAP were subcloned into pkmyc, METTL14 was subcloned into pCMV5-HA, and WTAP (human and mice), ERK1, ERK2, and USP5 were cloned into pCMV5-Flag. All mutants were generated using the QuikChange Site-Directed Mutagenesis Kit (Stratagene). The annealed siMETTL3 (TRCN0000289742) and siUSP5 (TRCN0000306799) specific targeted sequence was inserted into Tet-pLKO (Addgene, #21915). Myc-METTL3-T2A-Flag-WTAP was cloned into pCDH-CuO-MCS-EF 1α-RFP (System Biosciences, QM512B-1). pCDG-EF1α-CymR-T2A-Neo (QM400PA-2) for the cumate suppressor was ordered from System Biosciences.

Transfection and Virus Production. For transient transfection, cells were transfected by Lipofectamine 2000 as previously described (Lee et al., 2007). For lentivirus production, a lentiviral construct (pCDH-CMV-MCS-EF1α-RFP plasmids for overexpressing circRNA-GFP, Tet-pLKO for inducible knockdown of METTL3 or USP5, pCDG-EF1α-CymR-T2A-Neo for cumate repressor, or pCDH-CuO-MCS-EF1α-RFP for inducible overexpression of METTL3-T2A-WTAP) together with pMD2.G and psPAX2 were co-transfected into 293TN cells (System Biosciences). Viruses were concentrated by the PEG-it Virus Precipitation Solution and used for infecting cells in the presence of TransDux (System Bioscience). Pools of stable transfectants were selected by antibiotics or sorted by flow cytometry. Doxycycline (0.5 μg/mL) was used to induce shRNA while cumate (50 μg/mL) was used to induce shRNA-resistant cDNA expression.

Luciferase Reporter Assay. The luciferase plasmid LightSwitch 3′UTR Reporter, containing the Nanog 3′UTR or random negative control R01_3′UTR (Switchgear Genomics) was co-transfected with the m⁶A writer complex and ERK-activated kinase into HeLa cells for two days. Luciferase expression was measured using the Luciferase Assay System according to the commercial protocol (Promega). Nanog 3′UTR luciferase activity was normalized to cells transfected with R01_3′UTR.

Flow Cytometry. Flow cytometry analysis was conducted on BD LSR Fortessa and cell sorting was conducted on BD FACSAria Fusion. For alkaline phosphatase (Wiederschain et al.) staining, mESCs were incubated with fluorescent AP live stain (Sigma) for 30 min. For SSEA-1 expression, cells were disaggregated with trypsin, blocked with TruStain FcX (Biolegend) then incubated with anti-S SEA-1 (Biolegend) in cell staining buffer (Biolegend)

CRISPR Screen. The genome-wide CRISPR-Cas9 gene knockdown screen was accomplished using GeCKOv2 gene knockout library following published protocol (Joung et al., 2017). Briefly, the GECKOV2 library was amplified in Endura electrocompetent cells (Lucigen) then co-transfected with pMD2.G and psPAX2 into 293TN cells to produce a lentiviral library. HeLa-circGFP cells were infected at 0.3 MOI for 3 days, then selected with 2 μg/ml puromycin for 1 week before flow cytometry sorting. Genomes of harvested cells were extracted by Quick-gDNA MidiPrep (Zymo). sgRNA after PCR amplification were sent to the University of Chicago Genomics Facility to be sequenced on Illumina HiSeq 4000 in single-end read mode. RIGER was used to analyze the sequencing results. To obtain the ranked difference plot, sgRNAs were ranked according to the difference between number of reads in low and high GFP populations. The top 1% of the sgRNAs that ranked with the greatest difference were selected for gene ontology enrichment analysis.

Immunoprecipitation, immunoblotting, and in vitro kinase assay. Immunoprecipitation (IP) and immunoblotting (IB) were performed as previously described (Sun et al., 2016). In brief, protein samples were isolated from respective cells by lysis in RIPA buffer (1% Triton X-100, 150 mM NaCl, 20 mM Na₂PO4, pH 7.4) containing Halt Protease and Phosphatase Inhibitor Cocktail (Thermo Scientific). Subsequently, a BCA assay (Thermo Scientific) was used to determine the protein concentrations. For IP, indicated antibody and protein A/G magnetic beads (Thermo Scientific) were incubated with lysate at 4° C. overnight followed by washing and elution with sample buffer. Equal amounts of protein were separated by SDS-PAGE followed by wet transfer to PVDF membranes. Blots were blocked with 5% non-fat milk or BSA and incubated with respective primary antibody at 4° C. overnight. Primary antibodies were detected by HRP-linked secondary antibodies (Cell Signaling) together with SuperSignal West Pico Plus chemiluminescent substrate (Thermo Scientific) and imaged in a FluorChem R system (ProteinSimple).

Phosphate-affinity gel electrophoresis was performed in gels containing 60 μM MnCl₂, and 30 μM acrylamide-pendant Phos-tag ligand (AAL-107, Wako Chemicals). For in vitro ERK2 kinase assays, recombinant full-length human ERK2 expressed in E. coli cells with an N-terminal GST tag and activated by MEK1, and N-terminal GST-tagged human METTL3/METTL14 complex expressed in Sf9 insect cells were purchased from SignalChem. Active ERK2 was serially diluted in Kinase Dilution Buffer III (SignalChem) and incubated with METTL3/METTL14 at 30° C. for 15 min. The reaction was stopped by the addition of the sample buffer then analyzed by IB.

Confocal Microscopy. For confocal microscopy, cells after treatments were fixed in 4% paraformaldehyde, permeabilized with 0.5% Triton X-100, blocked with 5% bovine serum albumin, incubated with primary antibodies overnight at 4° C. followed by incubation with the appropriate secondary antibody tagged with Alexa 488 or Alexa 568 (Molecular Probes). Nuclei were stained with 4′,6-diamidino-2-phenylindole (DAPI) before mounting. Confocal fluorescence images were captured using Olympus FV1000 confocal spectral microscope.

Mass Spectrometry. To identify phosphorylation sites of METTL3, METTL3 precipitated from 293T cells co-transfected with myc-METTL3 and B-Raf V600E was analyzed by SDS-PAGE. The protein band corresponding to METTL3 was excised and subjected to in-gel digestion with tryspin and chymotrypsin. Samples were analyzed by Ultimate Capillary LC system (Dionex) directly coupled to LTQ Orbitrap Mass Analyzer (Thermo Scientific) using the TopTenTM method. The data were searched on MASCOT (MassMatrix) against the human Swiss-Prot database. All the identified phospho-peptides were further confirmed by manually checking the results.

RNA Extraction and Real-Time qPCR. Total RNA was isolated using TRIzol (Invitrogen), and 200 ng of RNA was reversed transcribed into cDNA using PrimeScript RT Reagent Kit (Takara). Real-time qPCR was performed using the FastStart Essential DNA Green Master (Roche). HPRT1 was used as an internal control for normalization. Primers used in this study are listed below. For measuring RNA stability, cells were treated with 5 μg/ml actinomycin D and harvested at 0, 3, and 6 hr to determine the half-life of target mRNAs.

TABLE 2
Primers for RT-qPCR of mESC

| Gene | Forward | SEQ ID NO: | Reverse | SEQ ID NO: |
|---|---|---|---|---|
| Nanog | GAACGCCTCATCAATGCCTGCA | 1 | GAATCAGGGCTGCCTTGAAGAG | 2 |
| Zfp42 | GAGACTGAGGAAGATGGCTTCC | 3 | CTGGCGAGAAAGGTTTTGCTCC | 4 |
| Klf2 | CACCTAAAGGCGCATCTGCGTA | 5 | GTGACCTGTGTGCTTTCGGTAG | 6 |
| Sox2 | AACGGCAGCTACAGCATGATGC | 7 | CGAGCTGGTCATGGAGTTGTAC | 8 |
| Lefty1 | AGTCCTGGACAAGGCTGATGTG | 9 | GAGGTCTCTGACACCAGGAACC | 10 |
| Pou5f1 | CAGCAGATCACTCACATCGCCA | 11 | GCCTCATACTCTTCTCGTTGGG | 12 |
| Hprt | CTGGTGAAAAGGACCTCTCGAAG | 13 | CCAGTTTCACTAATGACACAAACG | 14 |
| GATA4 | GCCTCTATCACAAGATGAACGGC | 15 | TACAGGCTCACCCTCGGCATTA | 16 |
| GATA6 | ATGCGGTCTCTACAGCAAGATGA | 17 | CGCCATAAGGTAGTGGTTGTGG | 18 |
| SOX7 | TGAATGCCTTCATGGTGTGGGC | 19 | ACAGTGTCAGCGCCTTCCATGA | 20 |
| Hand1 | CAAAAAGACGGATGGTGGTCGC | 21 | TGCGCCCTTTAATCCTCTTCTCG | 22 |
| Snail | TGTCTGCACGACCTGTGGAAAG | 23 | CTTCACATCCGAGTGGGTTTGG | 24 |
| FLK1 | CGAGACCATTGAAGTGACTTGCC | 25 | TTCCTCACCCTGCGGATAGTCA | 26 |
| FGF5 | AGAGTGGGCATCGGTTTCCATC | 27 | CCTACAATCCCCTGAGACACAG | 28 |
| NeuroD1 | CCTTGCTACTCCAAGACCCAGA | 29 | TTGCAGAGCGTCTGTACGAAGG | 30 |
| OTX2 | TGAGGGAAGAGGTGGCACTGAA | 31 | GCCTCACTTTGTTCTGACCTCC | 32 |

LC-MS/MS quantification of m⁶A in poly(A) RNA. mRNA was extracted from the total RNA using 2 rounds of the Dynabeads mRNA purification kit. 100 ng of mRNA was digested by nuclease P1 (1U) in 20 μl of buffer containing 20 mM NH₄OAc (pH=5.3) at 42° C. for 2 h, followed by dephosphorylation with the addition of FastAP Thermosensitive alkaline phosphatase (1U) and FastAP buffer at 37° C. for 2 h. The sample was then diluted to 50 μl and filtered (0.22 μm pore size, 4 mm diameter, Millipore). 5 μl of the solution was separated by reverse phase ultra-performance liquid chromatography on a C18 column, followed by online mass spectrometry detection using an Agilent 6410 QQQ triple-quadrupole LC mass spectrometer in positive electrospray ionization mode. The nucleosides were quantified by using the nucleoside-to-base ion mass transitions of 282 to 150 (m⁶A) and 268 to 136 (A). Quantification was carried out by comparison with a standard curve obtained from pure nucleoside. The ratio of m⁶A to A was calculated based on the calibrated concentrations (Liu et al., 2018).

m⁶A-IP and m⁶A-seq. m⁶A-IP was performed using the EpiMark N6-Methyladenosine enrichment kit (NEB). Full length purified mRNA was used in m⁶A-IP-qPCR. For m⁶A-seq, mRNA was adjusted to 15 ng/μl in 100 μl and fragmented using a BioRuptor ultrasonicator (Diagenode) with 30 s on/off for 30 cycles. Input and RNA eluted from m⁶A-IP were used to prepare libraries with TruSeq Stranded mRNA Library Prep Kit (Illumina). Sequencing was carried out at the University of Chicago Genomics Facility on Illumina HiSeq 4000 in single-end read mode with 50 bp reads per read. Reads were aligned to the mycoplasma genome to assess contamination, followed by alignment to mouse genome version 10 (mm10) with HISAT2 v2.1.0 (Kim et al., 2015) with parameter −k 1.

The input library of m⁶A sequencing is used for comparing gene expression levels. DESeq2 (Love et al., 2014) was applied for differential expression between R-WT and R-3A2A mESCs with FDR<0.05 cutoff. m⁶A-seq data were analyzed as described before (De Jesus et al., 2019). m⁶A peak calling was performed using exomePeak R/Bioconductor package v 3.7 (Meng et al., 2013). Significant peaks with false discovery rate less than 0.05 were annotated to the RefSeq database (mm10). Homer v4.9.1 (Heinz et al., 2010) was used to search for the enriched motif in the m⁶A peak region where random peaks of 200 bp were used as background sequences for motif discovery. m⁶A peak distribution on the metagene was plotted by the R package Guitar (Cui et al., 2016).

Differential analysis of m⁶A methylation of patient samples was performed using the R package RADAR and MeRIPtools (Z. Z., M. Eckert, A. Zhu, A. Chryplewicz, D. F. D. J., D. Ren, R. N. K., E. Lengyel, C. H., and M. C.; unpublished observations). To summarize and visualize the m⁶A methylome data, principal component analysis (PCA) was performed using singular value decomposition approach implemented in R function (prcomp) on the logtransformed m⁶A-IP data. Pathway and gene ontology enrichment analysis were performed using WebGestalt (Liao et al., 2019) with default settings. Pathway enrichment terms were determined using WikiPathway and KEGG terms.

Cell Proliferation Assay. Cells were seeded in 96-well plates. The cell proliferation was assessed by SRB assay (Vichai and Kirtikara, 2006) at various time points. Briefly, cells after treatments were fixed with 10% TCA then stained with 0.05% SRB. After wash, bound SRB was solubilized with 10 mM Trizma base and measured at 515 nm.

Quantification of Histone Modifications. Histones were prepared from fresh cell pellets using Total Histone Extraction Kit (Epigentek). The efficiency of histone extraction was controlled by Coomassie blue staining and LB with anti-H3 antibody. Histone posttranslational modifications were quantified using the Histone H3 Modification Multiplex Assay Kit (Epigentek) following commercial protocol. Each histogram corresponds to the mean of 2 independent experiments and each measure was obtained using a pool of 100 ng of total histones from 2 independent extractions.

Statistical Analysis. Each experiment was performed at least three times, and representative data are shown. Data in the bar graphs are given as the mean±SEM. Means were checked for statistical difference using Student's t test, and p-values<0.05 were considered significant (*p<0.05, **p<0.01, ***p<0.001). For survival analysis, Kalpan-Meier analysis and a log rank test were applied.

Data Availability. The CRISPR screening and m⁶A-seq data generated during this study are available at GSE138776. The human data for the skin cutaneous melanoma (SKCM), was derived from the Cancer Genome Atlas (TCGA).

### RESOURCES

| | | |
|---|---|---|
| REAGENT or RESOURCE | SOURCE | IDENTIFIER |

Antibodies

| | | |
|---|---|---|
| Myc-tag (9B11) | Cell | 2276 |
| Mouse mAb | Signaling | |
| Flag (M2) | Sigma- | F1804 |
| Mouse mAb | Aldrich | |
| HA-tag (C29F4) | Cell | 3724 |
| Rabbit mAb | Signaling | |
| B-Raf (D9T6S) | Cell | 14814 |
| Rabbit mAb | Signaling | |
| HER2/ErbB2 (D8F12) | Cell | 4290 |
| XP Rabbit mAb | Signaling | |
| p-ERK1/2 (Thr202/ | Cell | 4370 |
| Tyr204) (D13.14.4E) | Signaling | |
| XP Rabbit mAb | | |
| GAPDH (D16H11) | Cell | 5174 |
| XP Rabbit mAb | Signaling | |
| β-Actin (8H10D10) | Cell | 3700 |
| Mouse mAb | Signaling | |
| METTL3 (EPR18810) | Abcam | ab195352 |
| Rabbit mAb | | |
| p-METTL3(S43) | Lifetein | customized |
| METTL14(D8K8W) | Cell | 51104 |
| Rabbit mAb | Signaling | |
| WTAP (4A10G9) | Proteintech | 60188 |
| Mouse mAb | | |
| USP5 (EPR10454) | Abcam | AM54170 |
| Rabbit mAb | | |
| Alexa 488- | BioLegend | 125609 |
| SSEA-1(MC480) | | |
| Mouse mAb | | |

Chemicals, Peptides, and Recombinant Proteins

| | | |
|---|---|---|
| Phos-tag | Wako | AAL-107 |
| Acrylamide | | |
| cycloheximide | Sigma- | 01810 |
| | Aldrich | |
| Actinomycin D | Sigma- | A1410 |
| | Aldrich | |
| MG-132 | Sigma- | 474787 |
| | Aldrich | |
| dabrafenib | Selleck | S2807 |
| | Chemicals | |
| PLX-4720 | Selleck | S1152 |
| | Chemicals | |
| PD0325901 | Selleck | S1036 |
| | Chemicals | |
| Trametinib | Selleck | S2673 |
| | Chemicals | |
| EOAI3402143 | MedchemExpress | HY-111408 |
| Vialinin A | Cayman | 10010519 |
| | Chemical | |
| tucatinib | Selleck | S8362 |
| | Chemicals | |
| Lapatinib | Selleck | S1028 |
| | Chemicals | |
| DAPI | Sigma- | D9542 |
| | Aldrich | |
| Alkaline Phosphatase | Sigma- | A14353 |
| Live Stain | Aldrich | |
| Active ERK2 | SignalChem | M28-10G-05 |
| METTL3/METTL14 | SignalChem | M323-380G-05 |
| Nuclease P1 from | Sigma- | N8630 |
| Penicillium citrinum | Aldrich | |
| FastAP Thermosensitive | Thermo | EF0654 |
| Alkaline Phosphatase | Scientific | |

Critical Commercial Assays

| | | |
|---|---|---|
| Dynabeads mRNA | ThermoFisher | 61006 |
| DIRECT Kit | | |
| EpiMark N6- | New England | E1610S |
| Methyladenosine | Biolabs | |
| Enrichment Kit | | |
| TruSeq Stranded mRNA | Illumina | 20020594 |
| Library Prep | | |
| Luciferase | ProMega | E1500 |
| Assay System | | |
| Prime Script RT | Takara | RR037B |
| Reagent Kit | | |
| EpiQuik Histone | Epigentek | P-3100-96 |
| H3 Modification | | |
| Multiplex Assay Kit | | |

Deposited Data

| | | |
|---|---|---|
| CRISPR Screening, m⁶A- | This | GSE138776 |
| MeRIP-seq, and RNA-seq | study | |

Experimental Models: Cell Lines

| | | |
|---|---|---|
| HeLa | American Type | CCL-2 |
| | Culture Collection | |
| | ATCC (ATCC) | |
| 293T | ATCC | CRL-3216 |
| 293TN | System | LV900A-1 |
| | Bioscience | |
| Mouse ESC | Provided by | N/A |
| METTL3KO | Dr. Yawei Gao | |
| A375 | Provided by | CRL-1619 |
| | Dr. Yu-Ying He | |
| CHL-1 | ATCC | CRL-9446 |
| MEL-624 | Provided by | N/A |
| | Dr. Yu-Ying He | |
| HCT116 | ATCC | CCL-247 |
| MCF7 | ATCC | HTB-22 |
| T47D | ATCC | HTB-133 |
| SKBR3 | ATCC | HTB-30 |
| BT474 | ATCC | HTB-20 |

Software and Algorithms

| | | |
|---|---|---|
| FlowJo | Treestar | https://www.flowjo.com |
| RIGER | | https://software.broadinstitute.org/ |
| | | GENE-E/download.html |
| HISAT2 | | https://ccb.jhu.edu/software/ |
| | | hisat2/index.shtml |
| DESeq2 | | https://bioconductor.org/ |
| | | packages/release/bioc/ |
| | | html/DESeq2.html |
| exomePeak R | | https://bioconductor.org/ |
| | | packages/release/bioc/ |
| | | html/exomePeak.html |
| Guitar | | https://bioconductor.org/ |
| | | packages/release/bioc/ |
| | | html/Guitar.html |
| RADAR | | https://scottzijiezhang.github.io/ |
| | | RADARmanual/Mannual.htm |
| MeRIPtools | | https://scottzijiezhang.github.io/ |
| | | MeRIPtoolsManual/index.html |
| Homer | | http://homer.ucsd.edu/ |
| | | homer/motif/ |
| Gene set enrichment | | http://software.broadinstitute.org/ |
| analysis (GSEA) | | gsea/index.jsp |
| GenePattern | | https://cloud.genepattern.org/ |
| | | gp/pages/index.jsf |

What is claimed is:

1. A composition for attenuating tumor cell viability comprising:
at least one deubiquitinase inhibitor;
at least one chromatin state modulator or at least one DNA damage repair modulator; and
a pharmaceutically acceptable carrier or excipient.

2. The composition of claim 1, wherein the at least one deubiquitinase inhibitor targets Ubiquitin Carboxyl-terminal Hydrolase 5 (USP5).

3. The composition of claim 1 or claim 2, wherein the at least one chromatin state modulator comprises a bromodomain and extraterminal domain (BET) inhibitor, a histone methyl transferase (HMT) inhibitor, and/or a poly(ADP-ribose) polymerase 1 (PARP1) inhibitor.

4. The composition of claim 1 or claim 2, wherein the at least one DNA damage repair modulator induces DNA damage and/or inhibits DNA repair.

5. The composition of any of claims 1 to 4, wherein inhibiting USP5 attenuates METTL3 protein stability and/or activity.

6. The composition of any of claims 1 to 5, wherein the at least one deubiquitinase inhibitor comprises EOAI3402143, vialinin, WP1130, mebendazole, PYR-41, gossypetin, formonectin, suramin, and combinations thereof.

7. The composition of any of claims 1 to 6, wherein the BET inhibitor comprises a thienotriazolodiazepine, OTX015, BET-d246, ABBV-075, I-BET 151, I-BET 762, CPI 203, PFI-1, RVX-208, Dinaciclib, and combinations thereof.

8. The composition of claim 7, wherein the thienotriazolodiazepine is JQ1.

9. The composition of any of claims 1 to 8, wherein the HMT inhibitor comprises chaetocin, GSK343, UNC199, SGC0946, F5446, Pinometostat, EPZ004777, EPZ005687, tazemestostat, JQEZ5, CPI-1205, EPZ001989, EBI-2511, PF-06726304, El1, GSK503, GSK126, CPI-169, ZLD 1039, SAH-EZH2, NSC 617989, CPI-169, CPL-360, EPZ6438, and combinations thereof.

10. The composition of any of claims 1 to 9, wherein the PARP inhibitor comprises olaparib, rucaparib, veliparib, talazoprib, AG-14361, INO-1001, A-966492, PJ34, Niraparib, UPF 1069, ME0328, Pamiparib, NMS-P118, E7449, picolinamide, Benzamide, Nu1025, Iniparib, AZD2461, BGP-15, and combinations thereof.

11. The composition of any of claims 1 to 10, wherein the at least one DNA damage repair modulator comprises bleomycin, 5-FU, ceralasertib (AZD6738), cisplatin, oxaliplatin, carboplatin, Cytoxan, and combinations thereof.

12. The composition of any of claims 1 to 11, wherein the composition comprises at least one deubiquitinase inhibitor and wherein the at least one chromatin state modulator is a BET inhibitor.

13. The composition of any of claims 1 to 12, wherein the composition comprises at least one deubiquitinase inhibitor and wherein the at least one chromatin state modulator is an HMT inhibitor.

14. The composition of any of claims 1 to 13, wherein the composition comprises at least one deubiquitinase inhibitor and wherein the at least one chromatin state modulator is a PARP inhibitor.

15. The composition of any of claims 1 to 14, wherein the composition comprises at least one deubiquitinase inhibitor and at least one DNA damage repair modulator.

16. A method of treating or preventing cancer in a subject comprising administering the pharmaceutical composition of any of claims 1 to 15.

17. A method of treating or preventing cancer in a subject, the method comprising administering a composition comprising:
at least one deubiquitinase inhibitor; and
at least one of a bromodomain and extraterminal domain (BET) inhibitor, a histone methyl transferase (HMT) inhibitor, a poly(ADP-ribose) polymerase 1 (PARP1) inhibitor and/or a DNA damage repair modulator.

18. The method of claim 17, wherein the composition further comprises a pharmaceutically acceptable carrier or excipient, and wherein the composition is administered to a subject diagnosed with cancer.

19. The method of claim 17 or claim 18, wherein the at least one deubiquitinase inhibitor comprises EOAI3402143, vialinin, WP1130, mebendazole, PYR-41, gossypetin, formonectin, suramin, and combinations thereof

20. The method of any of claims 17 to 19, wherein the BET inhibitor comprises a thienotriazolodiazepine, OTX015, BET-d246, ABBV-075, I-BET 151, I-BET 762, CPI 203, PFI-1, RVX-208, Dinaciclib, and combinations thereof.

21. The method of any of claims 17 to 20, wherein the HMT inhibitor comprises chaetocin, GSK343, UNC199, SG-C:0946, F5446, Pinometostat, EPZ004777, EPZ005687, tazemestostat, JQEZ5, CPI-1205, EPZ001989, EBI-2511, PF-06726304, El1, GSK503, GSK126, CPI-169, ZIA) 1039, SAII-EZ112 NSC 617989, CPI-169, CPI-360, EPZ6438, and combinations thereof.

22. The method of any of claims 17 to 21, wherein the PARP inhibitor comprises olaparib, rucaparib, veliparib, talazoprib, AG-14361, INO-1001, A-966492, PJ34, Niraparib, UPF 1069, ME0328, Pamiparib, NMS-P118, E7449, picolinamide, Benzamide, Nu1025, Iniparib, AZD2461, BGP-15, and combinations thereof.

23. The method of any of claims 17 to 22, wherein the composition attenuates METTL3 stability and/or activity and induces apoptosis of a cancer cell.

24. The method of any of claims 17 to 23, wherein the composition comprises at least one deubiquitinase inhibitor and at least one BET inhibitor.

25. The method of any of claims 17 to 24, wherein the combination of the at least one deubiquitinase inhibitor and the at least one BET inhibitor exhibits a synergistic effect on cancer cell viability.

26. The method of any of claims 27 to 25, wherein the composition comprises at least one deubiquitinase inhibitor and at least one HMT inhibitor.

27. The method of any of claims 17 to 26, wherein the combination of the at least one deubiquitinase inhibitor and the at least one HMT inhibitor exhibits a synergistic effect on cancer cell viability.

28. The method of any of claims 17 to 27, wherein the composition comprises at least one deubiquitinase inhibitor and at least one PARP inhibitor.

29. The method of any of claims 17 to 28, wherein the combination of the at least one deubiquitinase inhibitor and the at least one PARP inhibitor exhibits a synergistic effect on cancer cell viability.

30. The method of any of claims 17 to 29, wherein the combination of the at least one deubiquitinase inhibitor and the at least one DNA damage repair modulator exhibits a synergistic effect on cancer cell viability.

31. The method of any of claims 17 to 30, wherein the cancer is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, glioblastoma, lymphoma, and leukemia.


SEQUENCE LISTING (32 sequences)

SEQ ID NO: 1 - DNA, 22 bp, Artificial Sequence
  Synthetic
gaacgcctcatcaatgcctgca

SEQ ID NO: 2 - DNA, 22 bp, Artificial Sequence
  Synthetic
gaatcagggctgccttgaagag

SEQ ID NO: 3 - DNA, 22 bp, Artificial Sequence
  Synthetic
gagactgaggaagatggcttcc

SEQ ID NO: 4 - DNA, 22 bp, Artificial Sequence
  Synthetic
ctggcgagaaaggttttgctcc

SEQ ID NO: 5 - DNA, 22 bp, Artificial Sequence
  Synthetic
cacctaaaggcgcatctgcgta

SEQ ID NO: 6 - DNA, 22 bp, Artificial Sequence
  Synthetic
gtgacctgtgtgctttcggtag

SEQ ID NO: 7 - DNA, 22 bp, Artificial Sequence
  Synthetic
aacggcagctacagcatgatgc

SEQ ID NO: 8 - DNA, 22 bp, Artificial Sequence
  Synthetic
cgagctggtcatggagttgtac

SEQ ID NO: 9 - DNA, 22 bp, Artificial Sequence
  Synthetic
agtcctggacaaggctgatgtg

SEQ ID NO: 10 - DNA, 22 bp, Artificial Sequence
  Synthetic
gaggtctctgacaccaggaacc

SEQ ID NO: 11 - DNA, 22 bp, Artificial Sequence
  Synthetic
cagcagatcactcacatcgcca

SEQ ID NO: 12 - DNA, 22 bp, Artificial Sequence
  Synthetic
gcctcatactcttctcgttggg

SEQ ID NO: 13 - DNA, 23 bp, Artificial Sequence
  Synthetic
ctggtgaaaaggacctctcgaag

SEQ ID NO: 14 - DNA, 24 bp, Artificial Sequence
  Synthetic
ccagtttcactaatgacacaaacg

SEQ ID NO: 15 - DNA, 23 bp, Artificial Sequence
  Synthetic
gcctctatcacaagatgaacggc

SEQ ID NO: 16 - DNA, 22 bp, Artificial Sequence
  Synthetic
tacaggctcaccctcggcatta

SEQ ID NO: 17 - DNA, 23 bp, Artificial Sequence
  Synthetic
atgcggtctctacagcaagatga

SEQ ID NO: 18 - DNA, 22 bp, Artificial Sequence
  Synthetic
cgccataaggtagtggttgtgg

SEQ ID NO: 19 - DNA, 22 bp, Artificial Sequence
  Synthetic
tgaatgccttcatggtgtgggc

SEQ ID NO: 20 - DNA, 22 bp, Artificial Sequence
  Synthetic
acagtgtcagcgccttccatga

SEQ ID NO: 21 - DNA, 22 bp, Artificial Sequence
  Synthetic
caaaaagacggatggtggtcgc

SEQ ID NO: 22 - DNA, 23 bp, Artificial Sequence
  Synthetic
tgcgccctttaatcctcttctcg

SEQ ID NO: 23 - DNA, 22 bp, Artificial Sequence
  Synthetic
tgtctgcacgacctgtggaaag

SEQ ID NO: 24 - DNA, 22 bp, Artificial Sequence
  Synthetic
cttcacatccgagtgggtttgg

SEQ ID NO: 25 - DNA, 23 bp, Artificial Sequence
  Synthetic
cgagaccattgaagtgacttgcc

SEQ ID NO: 26 - DNA, 22 bp, Artificial Sequence
  Synthetic
ttcctcaccctgcggatagtca

SEQ ID NO: 27 - DNA, 22 bp, Artificial Sequence
  Synthetic
agagtgggcatcggtttccatc

SEQ ID NO: 28 - DNA, 22 bp, Artificial Sequence
  Synthetic
cctacaatcccctgagacacag

SEQ ID NO: 29 - DNA, 22 bp, Artificial Sequence
  Synthetic
ccttgctactccaagacccaga

SEQ ID NO: 30 - DNA, 22 bp, Artificial Sequence
  Synthetic
ttgcagagcgtctgtacgaagg

SEQ ID NO: 31 - DNA, 22 bp, Artificial Sequence
  Synthetic
tgagggaagaggtggcactgaa

SEQ ID NO: 32 - DNA, 22 bp, Artificial Sequence
  Synthetic
gcctcactttgttctgacctcc